SEQUENCE LISTING (78 sequences)

SEQ ID NO: 1 - protein, 393 aa, unidentified [sequence omitted]

SEQ ID NO: 2 - DNA, 306 bp, synthetic construct
  source 1..306: mol_type = other DNA
atggatggatggtgtcctgggagagaccgtcggacagaggaagaaaatttccgcaaaaaagaagagcattgcccggagctgcccccagggagtgcaaagagagcactgcccaccagcacaagctcctctccccagcaaaagaaaaaaccactcgatggagaatatttcacccttaagatccgtgggcgtgagcgcttcgagatgttccgagagctgaatgaggccttggaattaaaggatgcccgtgctgccgaggagtcaggagacagcagggctcactccagctacccgaagatagttagttag

SEQ ID NO: 3 - protein, 101 aa, synthetic construct
MDGWCPGRDRRTEEENFRKKEEHCPELPPGSAKRALPTSTSSSPQQKKKPLDGEYFTLKIRGRERFEMFRELNEALELKDARAAEESGDSRAHSSYPKIVS

SEQ ID NO: 4 - DNA, 387 bp, unidentified
  source 1..387: mol_type = other DNA
atggccgctgctgtggaagccctgtacgtggtgcttgaaagagagggcgccatcctgcctagacaagagggcttttctggcgtgtacgtgttcttcagccccatcaacttcgtgatccctccaatgggcgccgtgatgctgagcctgagactgagagtgtgtatccctcctggctacttcggccggtttctggccctgaccgatgtgaaccagcctgacgtgttcaccgagagctacatcatgacccctgacatgaccgaggaactgagcgtggtgctgttcaaccacggcgaccagttcttttatggccacgccggaatggccgtcgtgcggctgatgctgatcagagtggtgtttcccgtcgtccggcaggccagcaatgtttga

SEQ ID NO: 5 - protein, 128 aa, unidentified
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 6 - protein, 128 aa, unidentified
MAAAVEALYVVLEREGAILPRQEGFSGVYVFFSPINFVIPPMGAVMLPLRLRVCIPPGYFGRFLALTDVNQPDVFTESYIMTPDMTEELSVVLFNHGDQFFYGHAGMAVVRLMLIRVVFPVVRQASNV

SEQ ID NO: 7 - protein, 128 aa, unidentified
MAAAVEALYVVLEREGAILPRQEGFSGVYVFFSPINFVIPPMGAVMLSLRLRVCIPPGYFGRFLALTDVNQPDVFTESYIMTPDMIEELSVVLFNHGDQFFYGHAGMAVVRLMLIRVVFPVVRQASNV

SEQ ID NO: 8 - protein, 125 aa, unidentified
MADEALYVYLEGPGATLPEQQQRNNYIFYSPVPFTLYPRGVALLYLKLSIIIPRGYVGCFFSLTDANMSGLYASSRIIHAGHREELSVLLFNHNDRFYEGRAGDPVACLVMERLIFPPVRQATMI

SEQ ID NO: 9 - protein, 124 aa, unidentified
MDAQVLYVFLEGAGALLPVQKGSNYIFYAPANFVLHPHGVALLELRLSIVVPQGFIGRFFSLTDANVPGVYASSRIIHAGHREGLSVMLFNHNVSFYNGRAGDPVACLVLERVIYPPVRQASMV

SEQ ID NO: 10 - protein, 128 aa, unidentified
MAAAVEALYVVLEREGAILPRQEGFSGVYVFFSPINFVIPPMGAVMLSLRLRVCIPPGYFGRFLALTDVSQPDVFTESYIMTPDMTEELSVVLFNHGDQFFYGHAGMAVVRLMLIRVVFPVVRQASNV

SEQ ID NO: 11 - protein, 125 aa, unidentified
MADEALYVYLEGPGATLPEQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 12 - protein, 125 aa, unidentified
MAESLYAFIDSPGGIAPVQEGTSNRYTFFCPESFHIPPHGVVLLHLKVSVLVPTGYQGRFMALNDYHARDILTQSDVIFAGRRQELTVLLFNHTDRFLYVRKGHPVGTLLLERVIFPSVKIATLV

SEQ ID NO: 13 - protein, 125 aa, unidentified
MADEALYVYLEGPGATLPEQQQRNNYIFYSPVPFTLYPRGVALLYLRLSIIIPRGYIGCFLSLTDANMFGLYASSRIIHAGHREELSVLLFNHDDRFYEGRAGDPVACLVMERLIYPPVRQATLI

SEQ ID NO: 14 - protein, 126 aa, unidentified
MADEALYVYFRGPGATLPEQQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 15 - protein, 127 aa, unidentified
MAALQALYVYFKGPGAMLPEQEGYSNAYVLFSPANFVIPPHGVVLLYLHIAVDIPPGYLGTLFSLSDMNARGVFVGAETLYPGSRMELSVLLFNHSDVFCDVRAKQPVARLLLSRVIFPPVRQASLL

SEQ ID NO: 16 - protein, 125 aa, unidentified
MAESLYAFIDSPGGIAPVQEGTSNRYNFFCPQSFHIPPHGVVLLHLKVSVLVPTGYQGRFMALNDYHARDILTQSDVIFAGRRQELTVLLFNHTDRFLYVRKGHPVGTLLLERVIFPSVKIATLV

SEQ ID NO: 17 - protein, 123 aa, unidentified
MAEVLYVILEGPGARLPVQEGNNYIFYAPVDFTLHPRGVALLHLRLSIIVPRCYIGRFFSLTDTNTSGLYASSQIIFAAHQQPLSVMLFNHTDRFYEGRVGDPVACLVLERVIYPSVRQASMM

SEQ ID NO: 18 - protein, 125 aa, unidentified
MAESLYAFIDSPGGIAPVQEGSSNRYNFFCPESFHIPPHGVVLLHLRVSVLIPTGYQGRFMALNDYHARGILTQSDVIFAGRRHELTVLLFNHTDRFLYVREGHPVGTLLLERVIFPSVRLATLV

SEQ ID NO: 19 - protein, 125 aa, unidentified
MAESLYAFIDSPGGIAPVQEGTSNRYTFFCPESFHIPPHGVVLVHLRVSVLIPNGYQGRFMALNDYHSRGILTQSDVIFAGRRQELTVLLFNHTDRFLYVREGHPVGTLLLERVIFPSVRLATLV

SEQ ID NO: 20 - protein, 125 aa, unidentified
MAESLYAFIDSPGGIAPVQEGASNRYTFFCPESFHIPPHGVILLHLRVSVLVPTGYQGRFMALNDYHARGILTQSDVIFAGRRHDLSVLLFNHTDRFLYVREGHPVGTLLLERVIFPSVRLATLV

SEQ ID NO: 21 - protein, 125 aa, unidentified
MAESLYAFIDSPGGIAPVQEGASNRYIFFCPESFHIPPHGVILLHLRVSVLVPTGYQGRFMALNDYHARGILTQSDVIFAGRRHDLSVLLFNHTDRFLYVREGHPVGTLLLERVIFPSVRIATLV

SEQ ID NO: 22 - protein, 125 aa, unidentified
MADEALYVYLDGPGATLPEQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 23 - protein, 125 aa, unidentified
MAHEALYVYLEGPGATLPEQQQRNNYIFYSPVPFTLYPRGVALLYLRLSIIIPRGYIGCFLSLTDANMFGLYASSRIIHAGHREELSVLLFNHDDRFYEGRAGDPVACLVMERLIYPPVRQATLI

SEQ ID NO: 24 - protein, 125 aa, unidentified
MAESLYAFIDSPGGIAPVQEGASNRYIFFCPESFHIPPHGVILLHLRVSVMVPTGYQGRFMALNDYHARGILTQSDVIFAGRRHDLSVLLFNHTDRFLYVREGHPVGTLLLERVIFPSVRIATLV

SEQ ID NO: 25 - protein, 125 aa, unidentified
MAESLYAFIDSPGGIAPVQEGTSNRYDFFCPESFHIPPHGVVLLHLRVSVLIPTGYQGRFMALNDYHARGILTQSDVIFAGRRHELTVLLFNHTDRFLYVREGHPVGTLLLERVIFPSVRLATLV

SEQ ID NO: 26 - DNA, 453 bp, unidentified
  source 1..453: mol_type = other DNA
atgaccaccagcggcgtgcccttcggcatgacactcagacctaccagaagccggctgagcagaagaaccccttacagcagagacaggctgcctccattcgagacagagacacgggccaccatcctggaagatcaccctctgctgcccgagtgtaacaccctgaccatgcacaacgcctggacaagcccatctcctccagtgaaacagccccaagtgggacagcagcctgttgctcagcagctggacagcgacatgaacctgtctgaactgcccggcgagttcatcaacatcaccgacgagagactggcccggcaagagacagtgtggaacatcacccctaagaacatgagcgtgacccacgacatgatgctgttcaaggccagcagaggcgagcggacagtgtacagcgtttgttgggaaggcggcggacggctgaataccagagtgctgtaa

SEQ ID NO: 27 - protein, 150 aa, unidentified
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 28 - skipped

SEQ ID NO: 29 - protein, 150 aa, unidentified
MTTSGVPFGMTLRPTRSRLSRRTPYSRDRLPPFETETRATILEDHPLLPECNTLTMHNAWTSPSPPVEQPQVGQQPVAQQLDSNMNLRELPGEFINITDERLARQETVWNITPKNMSVTHDMMLFKASRGERTVYSVCWEGGGRLNTRVL

SEQ ID NO: 30 - protein, 150 aa, unidentified
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 31 - protein, 141 aa, unidentified
MSGSNSIMTRLRARSTSCARHHPYTRAQLPRCEENETRASMTEDHPLLPDCDTMTMHSMTVIQTPESHPQQLDCESALKDYPDAFLCITDPRLARSETVWNVESKTMSISNGIQMFKAVRGERLVYSVKWEGGGKITTRIL

SEQ ID NO: 32 - protein, 141 aa, unidentified
MSGNSSIMTRSRTRLALSRHHPYQPPATLPRCEETESRASLVEDHPVLPDCDTLSMHNITVIPITEDSPQLLNCEVQMQECPEGFISLTDPRLSRSETVWNVEIKTMSITNSIQMFKAVRGERIVYSMRWEGGGKITTRIL

SEQ ID NO: 33 - protein, 150 aa, unidentified
MTTSGVPFGMTLRPTRSRLSRRTPYSRDRLPPFETETRATILEDHPLLPECNTLTMHNAWTSPSPSVKRPQVGQQPVAQQLDSDMNLSELPGEFINITDERLARQETVWNITPKNMSVTHDMMLFKASRGERTVYSVCWEGGGRLNTRVL

SEQ ID NO: 34 - protein, 141 aa, unidentified
MSGSNSIMTRLRARSTSCARHHPYTRAQLPRCEENETRASMTEDHPLLPDCDTMTMHSMTVIQTPESHPQQLDCESALKDYPDAFLCITDPRLARFETVWNVESKTMSICNGIQMFKAVRGERLVYSVKWEGGGKITTRIL

SEQ ID NO: 35 - protein, 141 aa, unidentified
MSGSNSIMTRLRARSTSCARHHPYTRAQLPRCEENETRASMTEDHPLLPDCDTMTMHSMTVIQTPESQPQQLDCESALKDYPDAFLSITDPRLARSETVWNVESKTMSISNGIQMFKAVRGERLVYSVKWEGGGKITTRIL

SEQ ID NO: 36 - protein, 141 aa, unidentified
MSGSNSIMTRLCARSTSCARHHPYTRAQLPRCEENETRASMTEDHPLLPDCDTMTMHSMTVIQTPESHPQQLDCESALKDYPAGFLSITDPRLARYETVWNVESKTMSISNGIQMFKAVRGERLVYSVKWEGGGKITTRSL

SEQ ID NO: 37 - protein, 126 aa, unidentified
MQRNRRYPYRLAPYGKYPLPPCEKEMRASLFGPENSSVSECNSLTLHNVINMDLVLDGESYLSDCVGEGFVSIIDHRFARKETIWTVTPKNLSRNMHMQLFSAIKGERVVYKIKWEGGGSLTTRIV

SEQ ID NO: 38 - protein, 130 aa, unidentified
MQTEDQSSLLRHHPYRRARLPRSDEETRASLTEQHPLLPDCDHADYHNTVTLDCEARLDDFSEDGFISITDPRLARQETVWIIDPKLSSRTNQNIPLFKATRAERTVYTVKWAGGGRLTTRAGVKINKDT

SEQ ID NO: 39 - protein, 130 aa, unidentified
MQTEIQSSSLRHHPYRRARLPRSDEETRASLTEQHPLLPDCDHADYHNTVTLDCEARLDDFSEDGFISITDPRLARQETVWIIDPKSSSRTNQNISLFKATRAERTVYTVKWAGGGRLTTRAGVKINKDT

SEQ ID NO: 40 - protein, 130 aa, unidentified
MSTEEQSSSLRHHPYRRARLPRCEEETRASLTEQHPLLPDCDHADYHNTVTLDCEARLDDFSEDGFISITDPRLARQETVWIIDTKTSSRTNNNIPLFKATRAERIVYTVKWAGGGRLTTRAGVKINKDT

SEQ ID NO: 41 - protein, 130 aa, unidentified
MSTEEQSSSLRHHPYRRARLPRCEEETRASLTEQHPLLPDCDHADYHNTVTLDCEARLDDFSEDGFISITDPRLARQETVWIIDPKSSSRTNQNISLFKATRAERTVYTVKWAGGGRLTTRAGVKINKDT

SEQ ID NO: 42 - protein, 130 aa, unidentified
MQTEDQSSLLRHHPYRRARLPRSDEETRASLTEQHPLLPDCDHADYHNTVTLDCEARLDDFSEDGFISITDPRLARQETVWIIDPKFSSRTNQNIPLFKATRAERIVYTVKWAGGGRLTTRAGVKINKDT

SEQ ID NO: 43 - protein, 130 aa, unidentified
MQTEDQSSLLRHHPYRRARLPRSDEETRASLTEQHPLLPDCDHAEYHNTVTLDCEARLDDFSEDGFISITDPRLARQETVWLIDTKSSSRANQNIPLFKATRAERIVYTVKWAGGGRLTTRAGVKINKDT

SEQ ID NO: 44 - protein, 130 aa, unidentified
MSTEEQSSSLRHHPYRRARLPRCEEETRASLTEQHPLLPDCDHADYHNTVTLDCEARLDDFSEDGFISITDPRLARQETVWIIDSKSSSRTNQNIPLFKATRAERIVYTVKWAGGGRLTTRAGVKINKDP

SEQ ID NO: 45 - protein, 130 aa, unidentified
MSTEEQSSSLRHHPYRRARLPRCDEETRASLTEQHPLLPDCDHADYHNTVTLDCEARLDDFSEDGFISITDPRLARQETVWIIDPKSSSRTNQNIFLFKATRAERIVYTVKWAGGGRLTTRAGVKINKDT

SEQ ID NO: 46 - protein, 141 aa, unidentified
MSGSNSIMTRLRARSTSCARHHPYTRAQLPRCEENETRASMTEDHPLLPDCDTMTMHSMTVIQTPESHPQQLDCESALKDYPDAFLSITDPRLARSETVWNVESKTMSISNGIQMFKAVRGERLVYSVKWEGGGKITTRIL

SEQ ID NO: 47 - protein, 130 aa, unidentified
MSTEEQSTSLRHHPYRRARLPRSEEETRASLTEQHPLLPDCDHAEYHNTVTLDCEARLEDFSEDGFISITDPRLARQETVWIIDTKSSSRTNQNIPLFKATRAERIVYTVKWAGGGRLTTRAGVKINKDT

SEQ ID NO: 48 - protein, 130 aa, unidentified
MSTEEQSSLLRHHPYRRARLPRCEEETRASLTEQHPLLPDCDHADYHNTVTLDCEARLDDFSEDGFISITDPRLARQETVWIIDTKSSSRSNNNIPLFKATRAERIVYTVKWAGGGRLTTRAGVKINKDS

SEQ ID NO: 49 - protein, 130 aa, unidentified
MSTEEQSSLLRHHPYRRARLPRCEEETRASLTEQHPLLPDCDHADYHNTVTLDCEARLEDFSEDGFISITDPRLARQETVWIIDTKSSSRTNQNIPLFKATRAERIVYTVKWAGGGRLTTRAGVKINKDT

SEQ ID NO: 50 - protein, 130 aa, unidentified
MSTEEQSSLLRHHPYRRARLPRCEQETRASLTEQHPLLPDCDHAEYHNTVTLDCEPRLEDFSEDGFISITDPRLARQETVWLIDTKTSSRTNQNIPLFKATRAERTVYTVKWAGGGRLTTRAGVKINKDT

SEQ ID NO: 51 - protein, 130 aa, unidentified
MQTEDQSSLLRHHPYRRARLPRCEEETRASLTEQHPLLPDCDHADYHNTVTLDCEARLEDFSEDGFISITDPRLARQETVWLIDTKSSSRTNQNIPLFKATRAERTVYTVKWAGGGRLTTRAGVKINKDT

SEQ ID NO: 52 - protein, 130 aa, unidentified
MSTEEQSSLLRHHPYRRARLPRCEEETRASLTEQHPLLPDCDHADYHNTVTLDCEARLDDFSEDGFISITDPRLARQETVWIIDPKSSSRTNQNISLFKATRAERIVYTVKWAGGGRLTTRAGVKINKDT

SEQ ID NO: 53 - protein, 130 aa, unidentified
MSTEEQSSLLRHHPYRRARLPRSDEETRASLTEQHPLLPDCDHADYHNTVTLDCEARLEDFSEDGFISITDPRLARQETVWIIDPKSSSRTNQNIPLFKATRAERIVYTVKWAGGGRLTTRAGVKINKDT

SEQ ID NO: 54 - protein, 141 aa, unidentified
MSGNNSIMTRLRARSTSCARHHPYTRAQLPRCEENETRASMTEDHPLLPDCDTMTMHSMTVIQTPESHPQQLDCESALKDYPDAFLCITDPRLARSETVWNVETKTMSISNGIQMFKAVRGERLVYSVKWEGGGKITTRIL

SEQ ID NO: 55 - protein, 130 aa, unidentified
MQTEIQSSSLRHHPYRRARLPRSDEETRASLTEQHPLLPDCDHADYHNTVTLDCEARLDDFSEDGFISITDPRLARQETVWIINPKSSSRTNENFPLFKATRAERIVYTVKWAGGGRMCTRAGVKINKDT

SEQ ID NO: 56 - protein, 130 aa, unidentified
MSTEEQSTLLRHHPYRRARLSRYDKETRASLTEQHPLLPDCDHADYHNTVTLDCEARLDDLSKDGFISITDPRLARQETVWIIDPKSSSRTNENFPLFKATRTERIVYTVKWAGGGRMCTRAGVKINKDT

SEQ ID NO: 57 - protein, 130 aa, unidentified
MQTEIQSSLLRHHPYRRARLPRSDEETRASLTEQHPLLPDCDHADYHNTVTLDCEARLDDFSEDGFISITDPRLARQETVWLIDTKSSSRANQNIPLFKATRAERIVYTVKWAGGGRLTTRAGVKINKDT

SEQ ID NO: 58 - protein, 130 aa, unidentified
MQTEIQSSSLRHHPYRRARLPRSDEETRASLTEQHPLLPDCDHADYHNTVTLDCEARLDDFSEDGFISITDPRLARQETVWIIDPKSNSRTNENISLFKATRAERTVYTVKWAGGGRLSTRAGVKINKDT

SEQ ID NO: 59 - protein, 130 aa, unidentified
MQTEIQSSSLRHHPYRRARLPRSDEETRASLTEQHPLLPDCDHADYHNTVTLDCEARLDDFSEDGFISITDPRLARQETVWIIDPKSSSRTNQNISLFKATRAERIVYTVKWAGGGRLTTRAGVKINKDT

SEQ ID NO: 60 - DNA, 885 bp, unidentified
  source 1..885: mol_type = other DNA
atgacaaccagcggcgtgcccttcggcatgacactgaggcctacaagaagccggctgagcagaagaaccccttacagcagagacagactgcctccattcgagacagagacacgggccaccatcctggaagatcaccctctgctgcccgagtgcaacaccctgacaatgcacaacgtgtcctacgtgcggggcctgccttgtagcgttggcttcacactgatccaagagtgggtcgtgccctgggacatggtgctgaccagagaggaactggtcatcctgcggaagtgtatgcacgtgtgcctgtgctgcgccaacatcgacatcatgaccagcatgatgatccacggctacgagagctgggccctgcactgtcactgttctagccctggcagcctgcagtgtattgctggtggacaggttctggccagctggttcagaatggtggtggacggcgccatgttcaaccagagattcatctggtacagagaggtggtcaactacaacatgcccaaagaagtgatgttcatgagcagcgttttcatgcggggcagacacctgatctacctgcggctttggtacgatggccacgtgggatctgtggtgcctgccatgagctttggctacagcgccctgcattgcggcatcctgaacaacatcgtggtgctgtgctgcagctactgcgccgatctgagcgagatcagagtgcggtgttgtgccagacggaccagacggctgatgctgagagccgtgcggatcattgccgaagagacaaccgccatgctgtactcttgccggaccgagagaagaaggcagcagttcatcagagccctgctccagcaccaccggcctatcctgatgcacgactacgacagcacccctatgtag

SEQ ID NO: 61 - protein, 294 aa, unidentified
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 62 - DNA, 1488 bp, unidentified
  source 1..1488: mol_type = other DNA
atggaaagacggaaccccagcgagagaggcgtgccagctggattttctggacacgccagcgtggaaagcggctgcgagacacaagaaagccctgccaccgtggtgttcagaccacctggcgataataccgatggcggagctgctgctgcagctggtggatctcaggcagcagctgcaggcgctgaacctatggaacctgagagcagacctggacctagcggcatgaatgtggtgcaggtcgccgagctgtatcccgagctgagaagaatcctgaccatcaccgaggatggccagggactgaagggcgtgaagagagaaagaggcgcctgcgaggccacagaggaagccagaaatctggccttcagcctgatgaccagacacagacccgagtgcatcaccttccagcagatcaaggacaactgcgccaacgagctggacctgctggcccagaagtacagcatcgagcagctgaccacctactggctgcaacccggcgacgatttcgaagaggccatcagagtgtacgccaaggtggccctcagacctgactgcaagtacaagatcagcaagctggtcaacatccggaactgctgctacatcagcggcaatggcgccgaggtggaaatcgacacagaggacagagtggccttccggtgcagcatgatcaacatgtggcctggcgtgctcggcatggatggcgtggtcattatgaacgtgcggttcacaggccccaacttcagcggcacagtgtttctggccaacaccaacctgatcctgcacggcgtgtccttctacggcttcaacaatacctgcgtggaagcctggaccgacgttcgcgttagaggctgcgccttctactgctgttggaagggcgtcgtgtgcagacccaagagcagagccagcatcaagaagtgcctgttcgagagatgcaccctgggcatcctgagcgagggcaacagcagagtcagacacaacgtggccagcgactgcggctgcttcatgctggttaagagcgtggccgtgatcaagcacaacatggtctgcggcaactgcgaggatagagccagccagatgctgacctgcagcgacggcaattgtcatctgctgaaaaccatccacgtggcctctcacagcagaaaggcctggcctgtgttcgagcacaatatcctgacacggtgctccctgcacctgggcaatagacggggagtgttcctgccttaccagtgcaacctgagccacaccaagatcctgctggaacccgagtccatgagcaaagtgaacctgaatggcgtgttcgacatgaccatgaagatctggaaagtgctgcgctacgacgagacacggaccagatgtagaccttgcgagtgtggcggcaagcacatcagaaaccagcctgtgatgctggacgtgaccgaggaactgaggcctgatcatctggtgctggcctgtaccagagccgagtttggcagctccgacgaggataccgat

SEQ ID NO: 63 - protein, 496 aa, unidentified
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 64 - DNA, 23 bp, unidentified
  source 1..23: mol_type = genomic DNA
gtcaccaatcctgtccctagtgg

SEQ ID NO: 65 - DNA, 23 bp, unidentified
  source 1..23: mol_type = genomic DNA
gtcccctccaccccacagtgggg

SEQ ID NO: 66 - DNA, 11 bp, unidentified
  source 1..11: mol_type = other DNA
aaagcccgggc

SEQ ID NO: 67 - DNA, 17 bp, unidentified
  source 1..17: mol_type = other DNA
ccaactccatcactagg

SEQ ID NO: 68 - DNA, 50 bp, unidentified
  source 1..50: mol_type = genomic DNA
ctgtcaccaatcctgtccctagtggccccactgtggggtggaggggacag

SEQ ID NO: 69 - DNA, 72 bp, unidentified
  source 1..72: mol_type = genomic DNA
agggaggcacgcctgtagctctgatgtcagatggcaatgtcgagatggcagtggccggtggggacagggctg

SEQ ID NO: 70 - DNA, 102 bp, unidentified
  source 1..102: mol_type = other DNA
actccctctctgcgcgctccctcgctcactgaggccgggcgaccaaaggtcgcccgacgcccgggctttgcccgggcggcctcagtgagcgagcgagcgcgc

SEQ ID NO: 71 - DNA, 101 bp, unidentified
  source 1..101: mol_type = other DNA
ctccctctctgcgcgctcgctcgctcactgaggccgggcgaccaaaggtcgcccgacgcccgggctttgcccgggcggcctcagtgagcgagcgagcgcgc

SEQ ID NO: 72 - DNA, 44 bp, unidentified
  source 1..44: mol_type = genomic DNA
cttgatcattctgacctctccaccccacagtgaggggaactgtg

SEQ ID NO: 73 - DNA, 54 bp, unidentified
  source 1..54: mol_type = genomic DNA
gttgggcgagatgaagctacactgtgaggtggaggtgatcagccggcacttgcc

SEQ ID NO: 74 - DNA, 54 bp, unidentified
  source 1..54: mol_type = genomic DNA
ttctgtcaccaatcctgtccctagtggccccactgtggggtggaggggacagat

SEQ ID NO: 75 - DNA, 51 bp, unidentified
  source 1..51: mol_type = genomic DNA
ttcttgatcattctgacctctccaccccacagtgaggggaactgtggtctc

SEQ ID NO: 76 - DNA, 53 bp, unidentified
  source 1..53: mol_type = genomic DNA
tccagccctgagcgggtctcctctaccccacagggtgggacctgttcagcttt

SEQ ID NO: 77 - DNA, 145 bp, unidentified
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 78 - DNA, 166 bp, unidentified
  source 1..166: mol_type = other DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgt

(19) United States
(12) Patent Application Publication
(10) Pub. No.: US 2025/0288617 A1
(43) Pub. Date: Sep. 18, 2025

(54) Gene Therapy

(71) Applicants: Ospedale San Raffaele S.r.l, Milan (IT); Fondazione Telethon ETS, Rome (IT)

(72) Inventors: Samuele Ferrari, Milan (IT); Aurelien Jacob, Milan (IT); Luigi Naldini, Milan (IT)

(21) Appl. No.: 18/859,829

(22) PCT Filed: Apr. 28, 2023
(86) PCT No.: PCT/EP2023/061387

§ 371 Date: Oct. 24, 2024

(30) Foreign Application Priority Data
Apr. 29, 2022 (GB) 2206346.5

(51) Int. Cl. A61K 35/28; C12N 5/0775; C12N 15/86

(52) U.S. Cl. CPC A61K 35/28; C12N 5/0662; C12N 15/86; C12N 2501/04; C12N 2501/125; C12N 2501/145; C12N 2501/22; C12N 2501/26; C12N 2750/14143

(57) ABSTRACT

Use of a combination of (a) cyclosporin H (CsH) or a derivative thereof, and (b) a p53 inhibitor and/or an adenoviral protein, or one or more nucleotide sequences encoding therefor, for increasing the efficiency of gene editing of an isolated population of cells when transduced by a viral vector and/or increasing the efficiency of transduction of an isolated population of cells by a viral vector.

### FIELD OF THE INVENTION

The present invention relates to compounds for improving the transduction of cells by viral vectors and/or improving gene editing of cells.

### BACKGROUND TO THE INVENTION

The haematopoietic system is a complex hierarchy of cells of different mature cell lineages. These include cells of the immune system that offer protection from pathogens, cells that carry oxygen through the body and cells involved in wound healing. All these mature cells are derived from a pool of haematopoietic stem cells (HSCs) that are capable of self-renewal and differentiation into any blood cell lineage. HSCs have the ability to replenish the entire haematopoietic system.

Haematopoietic cell transplantation (HCT) is a curative therapy for several inherited and acquired disorders. However, allogeneic HCT is limited by the poor availability of matched donors, the mortality associated with the allogeneic procedure which is mostly related to graft-versus-host disease (GvHD), and infectious complications provoked by the profound and long-lasting state of immune dysfunction.

Gene therapy approaches based on the transplantation of genetically modified autologous HSCs offer potentially improved safety and efficacy over allogeneic HCT. They are particularly relevant for patients lacking a matched donor.

The concept of stem cell gene therapy is based on the genetic modification of a relatively small number of stem cells. These persist long-term in the body by undergoing self-renewal, and generate large numbers of genetically “corrected” progeny. This ensures a continuous supply of corrected cells for the rest of the patient's lifetime. HSCs are particularly attractive targets for gene therapy since their genetic modification will be passed to all the blood cell lineages as they differentiate. Furthermore, HSCs can be easily and safely obtained, for example from bone marrow, mobilised peripheral blood and umbilical cord blood.

Efficient long-term gene modification of HSCs and their progeny requires a technology which permits stable integration of the corrective DNA into the genome, without affecting HSC function. Accordingly, the use of integrating recombinant viral systems such as γ-retroviruses, lentiviruses and spumaviruses has dominated this field (Chang, A. H. et al. (2007) Mol. Ther. 15:445-456). Therapeutic benefits have already been achieved in γ-retrovirus-based clinical trials for Adenosine Deaminase Severe Combined Immunodeficiency (ADA-SCID; Aiuti, A. et al. (2009) N. Engl. J. Med. 360:447-458), X-linked Severe Combined Immunodeficiency (SCID-X1; Hacein-Bey-Abina, S. et al. (2010) N. Engl. J. Med. 363:355-364) and Wiskott-Aldrich syndrome (WAS; Boztug, K. et al. (2010) N. Engl. J. Med. 363:1918-1927). In addition, lentiviruses have been employed as delivery vehicles in the treatment of X-linked adrenoleukodystrophy (ALD; Cartier, N. et al. (2009) Science 326:818-823), and for metachromatic leukodystrophy (MLD; Biffi, A. et al. (2013) Science 341:1233158) and WAS (Aiuti, A. et al. (2013) Science 341:1233151).

In addition to the use of retro- and lentiviral-based vectors, vectors derived from other viruses, such as adenoviruses and adeno-associated viruses (AAV), may also be utilised for the modification of haematopoietic stem and progenitor cells.

The scope of genetic engineering has recently broadened from gene replacement to targeted gene editing using engineered nucleases, which enable precise sequence modification of a locus of interest. Gene editing applications encompass targeted disruption of a gene coding sequence, precise sequence substitution for in situ correction of mutations and targeted transgene insertion into a predetermined locus. Gene editing is based on the design of artificial endonucleases that target a double-strand break (DSB) or nick into the sequence of interest in the genome. Cells repair the DSB through two major mechanisms, Non-Homologous End-Joining (NHEJ) or Homology Directed Repair (HDR), although other repair mechanisms might be eventually exploited. NHEJ often creates small insertions or deletions (“indels”) at the target site that may disrupt the coding sequence of a gene, whereas HDR can be exploited to precisely introduce a novel sequence at the target site by providing an exogenous template DNA bearing homology to the sequences flanking the DSB.

Multiple platforms of artificial endonucleases can be used to target a locus of interest, including Zinc Finger Nucleases (ZFNs), TAL effector nucleases (TALENs) and the more recently developed RNA-based CRISPR/Cas9 nucleases. Viral vectors are the most efficient delivery vehicle for a DNA template, for example, the AAV6 vector is able to achieve a high transduction efficiency in human primary cells, such as Hematopoietic Stem/Progenitor (HSPC) cells and T lymphocytes.

Despite recent advance in the generation of gene edited primary cells, several hurdles need to be solved before we can fully benefit from the predicted safety and precision of genetic engineering afforded by these new technologies.

A major issue is that gene editing in primary cells, and in particular in the primitive HSPC subset, is constrained by gene transfer efficiency and limited proficiency of homology directed DNA repair (HDR), likely due to HSC quiescence, to low levels of expression of the HDR machinery and conversely to high activity of the error-prone non homologous end joining (NHEJ) pathway. Thus, it will be crucial to enhance transduction efficiency and the gene editing efficiency in HSC, while preserving their long-term repopulating activity.

Similarly, the impact of adeno-associated virus (AAV) as a source of donor template for HDR-mediated gene editing remains poorly investigated, and no clinical application of this vector in HSPC has been reported yet. AAV dose-dependent toxicity has been observed, which is directly related to G-rich regions of ITRs that induce cells accumulation in early S-phase due to p53-mediated induction of apoptosis, as described in a hESCs model. Several studies have also reported frequent integration of fragmented or full-length AAV DNA throughout the genome of transduced cells both in cell lines and in vivo in post-mitotic cells (Schultz and Chamberlain, 2008, Molecular Therapy, 16:1189-1199). In this context, insertions near cancer genes were associated to the development of hepatocellular carcinoma in some mouse models and, more recently, to clonal expansion of hepatocytes in the long-term follow-up of gene therapy treated dogs (Dalwadi et al., 2021, Molecular Therapy, 29:680-690; Nguyen et al., 2020, Nature Biotechnology, 39 (1): 47-55).

Difficulties remain with the methods employed for the genetic modification of haematopoietic stem and progenitor cells. In particular, the multiple hits of high vector doses required and prolonged ex vivo transduction times associated with existing methods give rise to problems with survival of the transduced haematopoietic stem and progenitor cells during culture and potentially impact their biological properties. Furthermore, improvements in the engraftment of transduced cells will greatly benefit clinical applications.

### SUMMARY OF THE INVENTION

The present inventors have found that template delivery by Integrase-Defective Lentiviral Vector (IDLV) induced lower DNA load and less persistent DNA damage response allowing better preservation of clonogenic capacity and more efficient editing of long-term repopulating HSPCs. Because insertions of viral DNA fragments were much less frequent with IDLV and its LTRs are transcriptionally silent, its choice for template delivery significantly mitigates the adverse impact and genotoxic burden of HDR editing and should facilitate clinical translation of its application in HSPC gene therapy.

Furthermore, the inventors have developed an optimised protocol for transduction by a viral vector and gene editing of cells. In this regard, the inventors have surprisingly found that a combination of cyclosporin H (CsH) and a p53 inhibitor and/or adenoviral protein improves transduction and gene editing efficiencies of haematopoietic cells, haematopoietic stem cells and/or haematopoietic progenitor cells and improves the survival and/or engraftment of treated gene edited haematopoietic cells, haematopoietic stem cells and/or haematopoietic progenitor cells.

The inventors have developed an optimized HDR template delivery protocol for clinically relevant HSC sources, based on the use of integration defective lentiviral vector (IDLV), which substantially alleviates the burden of HDR editing and outperforms AAV in terms of editing efficiency in long-term repopulating HSCs. Further advantages of IDLV for HDR template delivery in HSPC editing are: i) the excellent safety and efficacy track record of LV-mediated HSPC gene therapy trials, which show robust polyclonal repopulation by vector treated HSPCs; ii) the larger cargo capacity, which enables more complex design of the therapeutic cassette (e.g., by including selector markers and purging unintended genetic addition or deletion at the target site) and, iii) the lower concern for immune rejection of ex vivo edited HSPCs upon presentation of residual viral antigens. HDR editing remains the most feasible and often unique approach to long-range gene correction when disease-causing mutations are several and spread over the gene sequence, or to target integration of a transgene cassette to safe harbors. The optimized IDLV-based gene editing protocol of the invention improves HDR-mediated gene editing efficiency in LT-HSCs and should facilitate safer and more effective clinical translation.

In one aspect, the invention provides the use of a combination of (a) cyclosporin H (CsH) or a derivative thereof, and (b) a p53 inhibitor and/or an adenoviral protein, or one or more nucleotide sequences encoding therefor, for increasing the efficiency of gene editing of an isolated population of cells when transduced by a viral vector and/or increasing the efficiency of transduction of an isolated population of cells by a viral vector.

In one embodiment, the use is an in vitro use or an ex vivo use.

In one embodiment, the combination comprises cyclosporin H (CsH) or a derivative thereof, a p53 inhibitor and an adenoviral protein, or one or more nucleotide sequences encoding therefor.

In one embodiment, the one or more nucleotide sequences are in the form of mRNA.

In one embodiment, the p53 inhibitor is a p53 dominant negative peptide.

In one embodiment, the p53 inhibitor directly inhibits p53.

In one embodiment, the p53 inhibitor is GSE56 or a variant thereof; pifithrin-a or a derivative thereof; or an siRNA, shRNA, miRNA or antisense DNA/RNA. Preferably, the p53 inhibitor is GSE56.

In one embodiment, the p53 inhibitor comprises or consists of an amino acid sequence having at least 70%, 75%, 80%, 85%, 90%, 95%, 96%, 97%, 98%, or 99% sequence identity to SEQ ID NO: 3.

In one embodiment, the p53 inhibitor comprises or consists of the sequence of SEQ ID NO: 3.

In one embodiment, the inhibitor is pifithrin-a or a derivative thereof. In one embodiment, the inhibitor is pifithrin-a cyclic. In one embodiment, the inhibitor is pifithrin-a p-nitro.

In one embodiment, the adenoviral protein is from an Adenovirus of serotype 5.

In one embodiment, the adenoviral protein is E4ORF1.

In one embodiment, the adenoviral protein is E4ORF6/7.

In one embodiment, the adenoviral protein comprises or consists of an amino acid sequence having at least 70%, 75%, 80%, 85%, 90%, 95%, 96%, 97%, 98%, or 99% sequence identity to SEQ ID NO: 27.

In one embodiment, the adenoviral protein comprises or consists of the sequence SEQ ID NO: 27.

In one embodiment, the combination comprises cyclosporin H (CsH) or a derivative thereof, and a polynucleotide (e.g. an mRNA) comprising nucleotide sequences encoding the p53 inhibitor and the adenoviral protein.

In one embodiment, the viral vector is an Integration-Defective Lentiviral Vector (IDLV).

In one embodiment, the viral vector comprises a template, such as a gene editing or homology directed repair (HDR) template. Preferably, the template is a DNA template. In one embodiment, the template comprises one or more exons of a CD40L gene (e.g. one or more of exons 2-5 of a CD40L gene). The template may, for example, not comprise exon 1 of CD40L. In one embodiment, the template encodes RAG1 (e.g. human RAG1).

In one embodiment, the cells are haematopoietic stem and/or progenitor cells (HSPCs). In one embodiment, the cells are CD34+CD133+CD90+ cells.

In one embodiment, the cells are T cells.

In one aspect, the invention provides a method of transducing a population of cells comprising the steps of:
i) contacting the population of cells with a combination of (a) cyclosporin H (CsH) or a derivative thereof, and (b) a p53 inhibitor and/or an adenoviral protein, or one or more nucleotide sequences encoding therefor; and
ii) transducing the population of cells with a viral vector.

In one embodiment, the method is an in vitro method or an ex vivo method.

In one embodiment, the method increases the efficiency of transduction of the population of cells and/or increases the efficiency of gene editing of the population of cells.

In one embodiment, the combination of the CsH or a derivative thereof, and the p53 inhibitor and/or the adenoviral protein, is used in combination with at least one additional transduction enhancer. Preferably, the at least one additional transduction enhancer is a PGE-2 (e.g. dmPGE-2) and/or lentiBOOST.

In one embodiment, the cells are contacted with CsH or derivative thereof prior to and/or at the same time as contact with the viral vector.

In one embodiment, the cells are contacted with CsH or derivative thereof and the at least one additional transduction enhancer prior to and/or at the same time as contact with the viral vector.

In one embodiment, the CsH or derivative thereof is at a concentration of about 1-50, 1-25, 1-20, 1-15, 1-10, 2-14, 3-13, 4-12, 5-11, 6-10 or 7-9 μM. Preferably, the concentration is about 8 μM.

In one embodiment, the cells are transduced with viral vector at an MOI of between about 10 to 250, such as 50 to 250, such as 100 to 200, preferably at an MOI of about 150.

In one embodiment, the invention provides a method of transducing a population of cells comprising the steps of:
i) contacting the population of cells with a combination of (a) cyclosporin H (CsH) or a derivative thereof, and (b) a p53 inhibitor and/or an adenoviral protein, or one or more nucleotide sequences encoding therefor;
ii) transducing the population of cells with a viral vector; and
iii) introducing gene editing machinery into the cells.

In one embodiment, the gene editing machinery may comprise a nuclease. In one embodiment, the nuclease is a site-directed nuclease. In one embodiment, the nuclease is a zinc finger nuclease (ZFNs), a transcription activator like effector nucleases (TALENs), meganucleases, or the clustered regularly interspaced short palindromic repeats (CRISPR)/Cas system.

The gene editing machinery (e.g. CRISPR/Cas system) may comprise one or more guide RNA complementary to at least one target gene in a cell, and an RNA-guided DNA endonuclease or nucleotide sequence encoding said endonuclease (e.g. Cas9 protein or a nucleotide sequence encoding a Cas9). In one embodiment, the gene editing machinery may be a CRISPR/Cas system. In one embodiment, the gene editing machinery is a CRISPR/Cas9 nuclease.

In one embodiment, the gene editing machinery is a ribonucleoprotein (RNP), comprising a nuclease (preferably a Cas protein (e.g. Cas9)) and a guide RNA (gRNA).

In one embodiment, the invention provides a method of transducing a population of cells comprising the steps of:
i) contacting the population of cells with a combination of (a) cyclosporin H (CsH) or a derivative thereof, and (b) a p53 inhibitor and/or an adenoviral protein, or one or more nucleotide sequences encoding therefor;
ii) transducing the population of cells with a viral vector; and
iii) introducing a site-directed nuclease into the cells.

In one embodiment, the site-directed nuclease is a ribonucleoprotein (RNP), comprising a nuclease (preferably a Cas protein (e.g. Cas9)) and a guide RNA (gRNA).

In one embodiment, the cells are contacted with RNP at a concentration of about 0.5 to 5 μM, such as about 1 to 4 μM, such as about 1.25 to 3 μM. Preferably, the RNP is at a concentration of about 1.25 to 2.5 μM.

In one embodiment, the gene editing machinery may be provided by one or more nucleotide sequence. Suitably, the nucleotide sequences encoding the gene editing machinery may be introduced to the cell sequentially or simultaneously.

In one embodiment, the p53 inhibitor and/or adenoviral protein, or one or more nucleotide sequences encoding therefor, may be contacted with the cell prior to and/or simultaneously with the introduction of gene editing machinery to the cell.

In one embodiment, gene-editing machinery and/or one or more nucleotide sequences encoding gene-editing machinery is introduced to the cell by electroporation, preferably by nucleofection. In one embodiment, gene-editing machinery and/or one or more nucleotide sequences encoding gene-editing machinery is introduced to the cell by transduction. In one embodiment, gene-editing machinery and/or one or more nucleotide sequences encoding gene-editing machinery is introduced to the cell by lipofection.

In one embodiment, gene-editing machinery and/or one or more nucleotide sequences encoding gene-editing machinery is introduced to the cell using lipid nanoparticles (LNPs).

In one embodiment, the gene-editing machinery, such as a site-directed nuclease (e.g. RNP), is introduced to a cell (e.g. by electroporation) after viral transduction for the delivery of the donor DNA template.

In one embodiment, the p53 inhibitor and/or adenoviral protein, or one or more nucleotide sequences encoding therefor, are present in the electroporation mixture.

In one embodiment, the nucleotide sequences (e.g mRNA) encoding p53 inhibitor and/or adenoviral protein, may be contacted with the cells at a concentration of about 50 to 300 μg/μl, such as about 100 to 250 μg/μl. In one embodiment, the nucleotide sequences (e.g mRNA) encoding p53 inhibitor and/or adenoviral protein, may be contacted with the cells at a concentration of about 250 μg/μl.

In one embodiment, the gene-editing machinery, such as a site-directed nuclease, is introduced into the cells about 10-14 hours, optionally about 12 hours after the beginning of the transduction step (e.g. first transduction step).

In one embodiment, the gene editing machinery, such as a site-directed nuclease, is introduced into the cells about 22-26 hours, optionally about 24 hours after the beginning of the transduction step (e.g. first transduction step).

In one embodiment, the population of cells is transduced by the viral vector in a single transduction step. The population of cells may, for example, not be subject to further transduction with viral vector before administration to a subject. In one embodiment, the population of cells is transduced by the viral vector in two transduction steps. The population of cells may be washed between the two transduction steps (e.g. to remove the viral vector).

In one embodiment, the second transduction step occurs after, such as between about 0 and 30 minutes, preferably 15 minutes, after introduction of the gene-editing machinery to the cells. In one embodiment, the second transduction step occurs immediately after introduction of the gene-editing machinery to the cells.

In one embodiment, the present invention provides a method of transducing a population of cells comprising the steps of:
i) contacting the population of cells with a cyclosporin H (CsH) or a derivative thereof,
ii) transducing the population of cells with a viral vector;
iii) contacting the population of cells with a p53 inhibitor and/or an adenoviral protein, or one or more nucleotide sequences encoding therefor;
iv) introducing gene editing machinery, such as a site-directed nuclease into the cells; and
v) optionally transducing the population of cells with a viral vector.

Suitably, the method steps are carried out in the order listed.

Suitably, step (i) and (ii) may be carried out concurrently or may partially overlap (e.g. the contact with the cyclosporin H (CsH) or a derivative thereof of step (i) may occur prior to and/or at the same time as the transduction with the viral vector of step (ii)).

Suitably, the population of cells may be further contacted with the cyclosporin H (CsH) or a derivative thereof prior to and/or at the same time as step (v).

In one embodiment, the population of cells is stimulated before transduction. In one embodiment, the population of cells is stimulated with early acting cytokines. In one embodiment, the population of cells is stimulated with expansion enhancers, such as a PGE2 (e.g. dmPGE2), UM171 and/or SR1. In one embodiment, the population of cells is stimulated for about 1-3 days before transduction. In one embodiment, the population of cells is stimulated for about 2-3 days before transduction. In one embodiment, the population of cells is stimulated for about 2-2.5 days before transduction. In one embodiment, the population of cells is stimulated for about 2 days before transduction. In one embodiment, the population of cells is stimulated for about 2.5 days before transduction.

In one embodiment, the present invention provides a method of transducing a population of cells comprising the steps of:
i) stimulating a population of cells;
ii) contacting the population of cells with a combination of (a) cyclosporin H (CsH) or a derivative thereof, and (b) a p53 inhibitor and/or an adenoviral protein, or one or more nucleotide sequences encoding therefor;
iii) transducing the population of cells with a viral vector; and
iv) introducing a site-directed nuclease into the cells.

In one embodiment, the present invention provides a method of transducing a population of cells comprising the steps of:
i) stimulating a population of cells;
ii) contacting the population of cells with cyclosporin H (CsH) or a derivative thereof,
iii) transducing the population of cells with a viral vector;
iv) contacting the population of cells with a p53 inhibitor and/or an adenoviral protein, or one or more nucleotide sequences encoding therefor;
v) introducing a site-directed nuclease into the cells; and
vi) optionally transducing the population of cells with a viral vector.

Suitably, the method steps are carried out in the order listed.

Suitably, step (ii) and (iii) may be carried out concurrently or may partially overlap (e.g. the contact with the cyclosporin H (CsH) or a derivative thereof of step (ii) may occur prior to and/or at the same time as the transduction with the viral vector of step (iii)).

Suitably, the population of cells may be further contacted with the cyclosporin H (CsH) or a derivative thereof prior to and/or at the same time as step (vi).

In one embodiment, the cyclosporin H (CsH) or a derivative thereof and, optionally, the additional transduction enhancers are present in both transduction steps.

In one embodiment, the population of cells is washed (e.g. with DPBS) after the first transduction step, for example to remove, or substantially remove, the viral vector and/or CsH or a derivative thereof (and optionally additional transduction enhancers) before introducing a site-directed nuclease into the cells.

In one aspect, the invention provides a combination of (a) cyclosporin H (CsH) or a derivative thereof, and (b) a p53 inhibitor and/or an adenoviral protein, or one or more nucleotide sequences encoding therefor, for use in a method of gene editing.

In one aspect, the invention provides a population of transduced cells, prepared according to the method of the invention.

In one aspect, the invention provides a pharmaceutical composition comprising the population of transduced cells prepared according the method of the invention.

In one aspect, the invention provides a method of treating or preventing a disease in a subject, comprising administering the population of cells according to the invention to the subject.

In one aspect, the invention provides a method of treating or preventing a disease in a subject, comprising administering a composition according to the invention to the subject.

In one aspect, the invention provides a population of cells according to the invention for use in a method of treating or preventing a disease in a subject.

In one aspect, the invention provides a composition according to the invention for use in a method of treating or preventing a disease in a subject.

In one aspect, the invention provides a method of gene therapy comprising the steps:
a) transducing a population of cells according to the method of the invention; and
b) administering the transduced cells to a subject.

In one embodiment, the transduced cells are administered to a subject as part of an autologous stem cell transplant procedure and/or an allogeneic stem cell transplant procedure.

In one aspect, the invention provides a method of gene editing a population of cells comprising the step of introducing gene-editing machinery and/or one or more nucleotide sequences encoding gene-editing machinery into the cells using lipid nanoparticles (LNPs).

In one embodiment, the method further comprises a step of transducing the population of cells with a viral vector. In one embodiment, the method further comprises a step of contacting the population of cells with a combination of (a) cyclosporin H (CsH) or a derivative thereof, and (b) a p53 inhibitor and/or an adenoviral protein, or one or more nucleotide sequences encoding therefor.

### DESCRIPTION OF THE DRAWINGS

FIG. 1. AAV ITRs induce p53 activation via MRN complex. a) Percentage of GFP+ cells in bulk and primitive (CD34+CD133+CD90+) human CB HSPCs 4 days (d) after editing the AAVS1 locus with AAV-delivered template (“RNP+ssAAV2/6”). Cells were transduced with 10-fold incremental vector doses measured as viral genomes (vg) per cell (n=3). Median. b) Fold change expression of CDKN1A (p21) relative to untreated cells (UT) 1 d after editing from experiments in FIG. 1a (n=4). Median. c) Number of colonies grown from seeded bulk HSPCs treated as indicated (n=1, 3, 3, 3). Median. d-e) Percentage of circulating hCD45+ (d) and GFP+ cells within the human graft (e) in mice transplanted with the outgrown progeny of starting-matched limiting cell doses of CB (left) or mPB (right) HSPCs after AAVS1 editing with the indicated treatments (n=10 mice/group for CB; n=5 mice/group for mPB). Please note that editing enhancers were used in the mPB experiment. Mean+s.e.m., Linear Mixed Effect model (LME) followed by post-hoc analysis where groups have been compared at fixed timepoints. Results are shown for the last timepoint. f) Percentage of GFP+ cells within CB HSPC subpopulations 4 d after AAVS1 editing in presence of “full” (5×10⁵ viral capsids/cell, equivalent to 2×10⁴ vg/cell) or “empty” (5×10⁷ viral capsids/cell) particles (n=3). Median. g) Fold change expression of CDKN1A (p21) over time relative to UT from experiments in FIG. 1f (n=3). Median. h) Number of colonies grown from seeded bulk HSPCs from experiments in FIG. 1f (n=3). Median. i-j) Percentage of circulating hCD45+ (i) and GFP+ cells within the human graft (j) in mice transplanted with the outgrown progeny of starting-matched limiting cell doses of CB HSPCs edited in AAVS1 as indicated (n=10, 9). Mean±s.e.m, LME followed by post-hoc analysis where groups have been compared at fixed timepoints (for FIG. 1i). Results are shown for the last timepoint. k) Fold change expression of CDKN1A (p21) relative to UT 1 d after AAVS1, IL2RG or CD40L editing with the indicated treatments (n=3). Median. I) Percentage of GFP+ cells within CB HSPC subpopulations 4 d after AAVS1 editing with 2×10⁴ ssAAV2/6 or scAAV2/6 (n=7). Median with 95% confidence interval (CI), Mann-Whitney test. m) Fold change expression of CDKN1A (p21) over time relative to UT from experiments in FIG. 1l (n=3). Median. n) Number of colonies grown from seeded bulk HSPCs from experiments in FIG. 1l (n=3). Median. o) Intracellular CG of the indicated AAV features retrieved over time from HSPCs edited with two doses 2×10³ vg/cell (left) and 2×10⁴ vg/cell (right) of ss or scAAV2/6 (n=2). To account for the low but detectable signal coming from extracellular cell-associated viral DNA, cells edited in presence of heat inactivated AAV were used as control for each condition and their CG values subtracted to the paired treatment samples (see Methods for further details). Median. p-q) Percentage of circulating hCD45+ (p) and GFP+ cells within the human graft (q) in mice transplanted with the outgrown progeny of starting-matched limiting cell doses of CB HSPCs edited in AAVS1 as indicated (n=5 mice/group). Mean±s.e.m, LME followed by post-hoc analysis where groups have been compared at fixed timepoints. Results are shown for the last timepoint. r) Number of unique indels retrieved by targeted AAVS1 deep sequencing in human splenocytes of mice in FIGS. 1d, i and p (n=9, 9, 10, 5). Median with quartiles, Kruskal-Wallis test with Dunn's multiple comparisons. s) Quantification of Nbs1 foci over time from HSPCs edited with the indicated treatments (n=5, 3, 5, 2, 5). Mean. t) Percentage of HDR-edited and GFP+ cells in bulk CB HSPC 4 d after AAVS1 editing in presence or absence of Ad5-E1B55K and/or Ad5-E4orf6 mRNAs (n=3, 4 for each treatment). Median. u) Representative fluorescence microscopy images of HSPCs from FIG. 1t. v) GFP relative fluorescence intensity (RFI) over the “RNP+ssAAV2/6” condition in bulk and primitive HSPCs 4 d after editing from experiments in FIG. 1t (n=3). Median. w) Fold change expression of APOBEC3H relative to UT 1 d after editing in experiments from FIG. 1t (n=4). Median. For all panels, “n” indicates biologically independent experiments or animals.

FIG. 2. AAV genome sensing leads to dose-dependent p53 activation independently from the manufacturing process and the serotype of ITRs. a) Percentage of HDR-edited alleles measured by 3′ targeted integration (TI) digital droplet (dd) PCR assay in bulk CB HSPCs from FIG. 1a 4 d after editing (n=3, 3, 2). Median. b) HSPC culture composition 4 d after editing in experiments from FIG. 1a (n=1, 3, 3, 3). Mean. c-d) Percentage of hCD45⁺ (c) and GFP⁺ cells within the human graft (d) at the end of the experiments (20 weeks, wks) within spleen and bone marrow of transplanted mice from FIG. 1d-e (n=9 mice/group). Median with quartiles, LME. e) Percentage of GFP⁺ cells within CB HSPC subpopulations 4 d after AAVS1 editing with AAV purified by CsCl gradient or AVB affinity chromatography (n=3). Median. f) Fold change expression of CDKN1A (p21) relative UT 1 d after editing in experiments from FIG. 2e (n=3). Median. g) Number of colonies grown from seeded bulk HSPCs from experiments in FIG. 2e (n=3). Median. h-i) Percentage of circulating hCD45⁺ (h) and GFP⁺ cells within human graft (i) in mice transplanted with the outgrown progeny of starting-matched limiting cell doses of CB HSPCs edited in AAVS1 as indicated (n=5 mice/group). Mean±s.e.m., LME followed by post-hoc analysis where groups have been compared at fixed timepoints. Results are shown for the last timepoint. j) Fold change expression of CDKN1A (p21) relative to UT 1 d after AAVS1 editing of CB HSPCs in presence of “full” (5×10⁵ viral capsids/cell, equivalent to 2×10⁴ vg/cell), “empty” (5×10⁷ viral capsids/cell) or “full+empty” (admixed at the indicated viral capsid ratios) AAV particles (n=5, 5, 5, 3, 3, 3). Median. k) Percentage of GFP⁺ cells within primitive HSPCs 4 d after editing in experiments from FIG. 2j (n=3). Median. I) Percentage of hCD45⁺ cells within organs of transplanted mice from FIG. 1i-j (n=10, 9). Median with quartiles, LME. m) Percentage of circulating hCD3⁺ cells in transplanted mice from FIG. 1i-j (n=10, 9). Mean±s.e.m., LME followed by post-hoc analysis where groups have been compared at fixed timepoints. Results are shown for the last timepoint. n) Percentage of GFP⁺ cells within CB HSPC subpopulations 4 d after AAVS1 editing with either ssAAV2/6 or ssAAV5/6 at indicated doses (n=4). Median. o) Percentage of HDR-edited alleles in bulk HSPCs 4 d after editing in experiments from FIG. 2n (n=4). Median. p) Silver stained SDS PAGE revealing AAV Cap proteins (VP1, VP2 and VP3) loaded with purified 2×10¹² vg and 4×10¹² vg of ssAAV2/6 and ssAAV5/6. q) Fold change expression of CDKN1A (p21) relative to UT 1 d after editing CB HSPCs from experiments in FIG. 2n (n=3). Median. r) Number of colonies grown from seeded bulk CB HSPCs from experiments in FIG. 2n (n=3). Median. s) Percentage of HDR-edited alleles in bulk CB HSPC 4 d after AAVS1 editing in experiments from FIG. 1l (n=3). Median. t) HSPC culture composition 4 d after editing in experiments from FIG. 1l (n=6). Mean±s.e.m. u) Fold change expansion of live CB HSPCs over time after indicated treatments (n=3). Median. v) Percentage of HDR- and NHEJ-edited alleles, quantified by ddPCR and deep sequencing respectively, in human splenocytes of mice in FIG. 1d, i, p (n=9, 9, 10, 5). w) Quantification of 53BP1 and γH2AX foci over time from HSPCs edited with the indicated treatments (n=5, 3, 5, 2, 5). Mean. x) GFP RFI over the ssAAV2/6-transduced condition in bulk and primitive CB HSPCs 4 d after treatments in presence of Ad5-E1B55K and/or Ad5-E4orf6 (n=2). y) Fold change expansion of live CB HSPCs over time after IL2RG editing with the indicated treatments (n=1, 1, 1, 2, 2). Median. z) Number of colonies grown from seeded bulk CB HSPCs in experiments from FIG. 2y (n=2, 2). Median. For all panels, “n” indicates biologically independent experiments or animals.

FIG. 3. Integration of transcription competent AAV ITR fragments at the nuclease on-target site. a) Heatmap showing alignment and normalized abundance (LogCPM) of reads bearing integrated DNA fragments (length>20 bp) across the AAV genome for each mouse from FIG. 1r (n=9, 9, 10, 5). UT samples sequenced and analyzed in parallel are shown (n=2). HA: homology arms. PolyA: bovine growth hormone polyadenylation signal. b) Percentage of AAVS1 alleles in human splenocytes from FIG. 3a carrying integrated DNA fragments. The proportion of mice in each group carrying at least one event of DNA fragment integration is shown above (n=9, 9, 10, 5). Median with quartiles, Kruskal-Wallis test with Dunn's multiple comparisons. c) Pie chart showing the proportion of fragments aligning to each region of the AAV genome within the total number of retrieved DNA fragments from experiments in FIG. 3a (n=28 alleles distributed across 23 mice). d) Percentage of alleles in human splenocytes from previously published experiments carrying integrated DNA fragments as in FIG. 3b. The proportion of mice in each group carrying at least one event of DNA fragment integration is shown above (n=23, 11, 15, 16). Median with quartiles, Kruskal-Wallis test with Dunn's multiple comparisons. e) Pie chart showing the proportion of fragments aligning on each region of the AAV or human genome within the total number of retrieved DNA fragments for experiments in FIG. 4e (n=53 alleles distributed across 65 mice). f-g) RFI over time of GFP⁺ CD4⁺ T cells (f) and mPB CD34⁺ HSPCs (g) from healthy donors transduced with the promoter-less ssAAV2/6 represented in FIG. 4j (n=4, 4, 4, 3 for T cells, n=3 for HSPCs). Cells transduced with AAV bearing GFP downstream a PGK promoter shown as reference. Median. For all panels, “n” indicates biologically independent experiments or animals.

FIG. 4. AAV ITR-2 harbor transcriptional activity in human hematopoietic cells. a) Fraction of DNA trapping events categorized by length ranges in the experimental setting of FIG. 3a (#1, n=28 events) and FIG. 4e (#2, n=53 events). b) Allele structure for two representative DNA trapping events (top: ITR fragment trapping with NHEJ deletion; bottom: aborted HDR integrating the molecular BAR). Fw: forward primer. Rv: reverse primer; BAR-Seq primer: primer used for BAR retrieval in previous analyses. c-d) Percentage of HDR- and NHEJ-edited alleles (c, median with 95% CI) and number of unique indels (d, median with quartiles) in human splenocytes of mice from previously published experiments (c: n=23, 10, 14, 16; d: n=23, 11, 15, 16). For FIG. 5d, Kruskal-Wallis with Dunn's multiple comparisons. e) Heatmap showing alignment and normalized abundance (LogCPM) of reads bearing integrated DNA fragments (length≥20 bp) across the AAV genome for each mouse from FIG. 4d (n=23, 11, 15, 16). UT samples sequenced and analyzed in parallel are shown (n=2). f) Percentage of BARs retrieved from analyses in FIG. 2d and recaptured (fragment length>45 bp, thus including the BAR-Seq primer binding site) or not in the BAR-Seq dataset from the same samples. The total number of analyzed events is shown above the bars. g-h) Percentage of HDR- and NHEJ-edited alleles, quantified by ddPCR and deep sequencing respectively, (g, median with 95% CI) and number of unique indels (h, median with quartiles) in human splenocytes of mice from previously published experiments (n=5, 9). Mann-Whitney test. i) Percentage of alleles in human splenocytes from FIG. 4h carrying integrated DNA fragments as in FIG. 3b. The proportion of mice in each group carrying at least one event of DNA fragment integration is shown above (n=5, 9). Median with quartiles. j) Alignment on the AAV genome of trapped DNA fragments in human CD4⁺ T cells engrafted in one NSG mouse from previously published experiments. k) Schematic of AAV constructs used to address putative ITR-2 transcriptional activity in human hematopoietic cells. SD: artificial splice donor site; SA: artificial splice acceptor site. Dashed red arrows indicate putative transcripts. Black arrows indicate primer binding sites used to amplify the spliced transcript (expected length=500 bp). I-m) Percentage over time of GFP⁺ CD4⁺ T cells (I) or mPB HSPCs (m) from different healthy donors transduced with the promoter-less AAV depicted in FIG. 4k (I: n=4, 4, 4, 3; m: n=3). Cells transduced with AAV bearing GFP downstream a PGK promoter shown as reference. Median. n) Capillary electrophoretic analysis showing transcript amplification at the expected molecular weight in three T cell donors (D) from FIG. 41. For all panels, “n” indicates biologically independent experiments or animals. FIG. 5. Unbiased genome wide retrieval of AAV IS reveals frequent integration events at the nuclease on- and off-target sites in edited LT-HSCs. a) Schematic representation of the PCR primer sets adopted for the retrieval of IS from AAV vectors used as repair template. Inner PCR primers adopted for nested amplification contained a barcode sequence for sample identification represented in blue. b) Number of AAV IS retrieved from hematopoietic organs of mice transplanted with HSPCs edited as indicated at AAVS1 (n=5, 3, 7, 8) or RAG1 (n=6). Median. c) Genome-wide distribution of AAV integrations retrieved from mice transplanted with HSPCs edited at AAVS1 (shown in red) and RAG1 (shown in black). The vast majority of AAV integrations were detected at the sgRNA target sites. d) Enlarged genomic view of AAV IS within the AAVS1 (top) and RAG1 (bottom) target sites. Genomic coordinates and scale are indicated. Black lines indicate the position of AAV IS. Black bars indicate homology arms and the protospacer sequence targeted by the different sgRNAs. e) Pie charts indicating the frequency of AAV features found at the IS upon editing AAVS1. Left graphs made from AAV insertions at validated on- and off-target sites and right graphs from AAV insertions outside the editing sites. f) Heatmap of the AAV 3′ITR secondary structure with red scale indicating the frequency of AAV insertions at the indicated nucleotide position for editing AAVS1 (left) or RAG1 (right). g) Pie charts as in FIG. 5e indicating the frequency of AAV features found at the IS upon editing RAG1. h-k) CG measured by the “ITR+cargo” ddPCR probe system within human splenocytes of mice from FIG. 1d, i, p (n=9, 9, 10, 5) (h), circulating hCD45⁺ cells in transplanted mice from previously published experiments (n=3, 4, 5, 4) (i), human hematopoietic lineages in transplanted mice from previously published experiments (n=3, 5, 5, 4) (j), human splenocyte of serially transplanted mice from previously published experiments (n=6, 6, 4, 4) (k). For FIG. 5h, median with quartiles. For FIG. 5i-k, median. For FIG. 5h, Kruskal-Wallis with Dunn's multiple comparisons. I) CG measured by the “ITR+cargo” or “ITR” ddPCR probe systems within human splenocytes of mice from FIG. 1d, i, p and UT samples. “ITR+cargo” and “ITR” values retrieved from the same sample are connected by a black line (n=20, 5, 9, 3). The dashed red line indicates the background noise signal threshold by the analysis of UT samples. m) “ITR” CG within human splenocyte of mice transplanted with HSPCs edited at different genomic loci (n=20, 23, 41). Median with quartiles. For all panels, “n” indicates biologically independent experiments or animals.

FIG. 6. Characterization and quantification of AAV IS in edited LT-HSCs. a) Schematic workflow of the bioinformatics procedures adopted for the identification and characterization of AAV IS. b) Percentage of hCD45⁺ in the hematopoietic organs of mice transplanted with HSPCs edited as indicated at AAVS1 (n=5, 3, 7, 8) or RAG1 (n=8). Mean±s.e.m. c) Percentage of GFP⁺ cells from AAVS1 experiments in FIG. 6b (n=5, 3, 7, 8). Median. d) Number of AAV IS retrieved by the PCR systems shown in FIG. 5a adopted for IS retrieval from mice transplanted with AAVS1-(left) or RAG1-(right) edited HSPCs (n=14, 17, 10, 15). e) Genomic view as in FIG. 5d of AAV IS within two predicted off-target sites of the LS sgRNA, LAMC3 (top) and LRR1 (bottom). Genomic coordinates and scale are indicated. Black lines indicate the position of AAV integration site and black bars indicate the off targeted protospacer sequence targeted by the sgRNAs. f) Enlarged genomic view as in FIG. 5d of AAV IS within the PGK gene. Black lines indicate the AAV IS and black bars indicate the PGK promoter sequence comprised within the AAV vector. g) Percentage of IS represented by the indicated number of genomes in IS dataset derived from AAVS1- and RAG1-edited human cells. Mean±s.e.m. h) Percentage of HDR-edited alleles in the bone marrow of mice transplanted with human bone marrow-derived HSPCs edited at RAG1 (n=6). Median. i) “ORI” CG within human splenocytes of mice from FIG. 1d, i, p and UT samples (n=2, 4, 5, 5). The dashed red line indicates the background noise signal threshold by the analysis of UT samples. Median. j-I) CG measured by the “ITR+cargo” ddPCR probe system within human splenocytes of mice from previously published experiments (n=7, 9, 9, 8) (j, Kruskal-Wallis with Dunn's multiple comparisons), human splenocytes of mice pooled samples from previously published experiments (n=43, 23) (k, Mann-Whitney), bone marrow purified hCD45⁺ CD34⁺ cells in serially transplanted mice from previously published experiments (n=4 mice/group) (I). For FIG. 6j, k: median with quartiles. For FIG. 61: median. For all panels, “n” indicates biologically independent experiments or animals.

FIG. 7. Optimized IDLV-based editing allows stealthier and more efficient HDR editing in human LT-HSCs. a) Number of unique indels retrieved by on-target AAVS1 sequencing in human BM cells of mice from FIG. 8c, d (n=10, 6, 10). Median with quartiles. Kruskal-Wallis test with Dunn's multiple comparisons. b) Percentage of alleles in human BM cells from FIG. 8c, d carrying integration of DNA fragments (length≥20 bp). The proportion of mice for each group carrying at least one event of DNA fragment integration is shown above (n=10, 6, 10). Median with 95% Cl. c) Schematic representation of the PCR primer sets adopted for unbiased genome wide retrieval of IS from IDLV used as repair template. Inner PCR primers adopted for nested amplification contained a barcode sequence for sample identification represented in blue. d) Number of IDLV IS retrieved from transplanted mice (n=4). Median. e) Genomic view of IDLV IS within the AAVS1 as in FIG. 5d. f) Pie charts indicating the frequency of IDLV features involved at the IS breakpoints. Left graph showing IDLV IS at validated on- and off-target sites and right graph showing IDLV IS outside the editing site. g) Percentage of GFP⁺ cells within mPB HSPC subpopulations 4 d after AAVS1 editing using IDLV for template delivery (“RNP+IDLV”) in presence or absence of CsH and GSE56/Ad5-E4orf6/7. Transduction performed once 24 or 12 hours before (1 hit) without or with a second one immediately after (2 hits) electroporation, using a multiplicity of infection (MOI) of 150 transducing units (TU^{293T})/cell per hit. Editing with the optimized ssAAV protocol performed in parallel (n=1, 2, 2, 5, 4, 5). Median. h) Percentage of HDR-edited alleles in bulk mPB HSPCs 4 d after AAVS1 editing with indicated optimized treatments (n=2). Median. i) Number of colonies grown from seeded mPB HSPCs edited with the indicated optimized treatments (n=6). Median with 95% CI. Friedman test with Dunn's multiple comparisons. j) Fold change expression of CDKN1A (p21) over time relative to UT after AAVS1 editing with IDLV or AAV optimized protocols in mPB HSPCs (n=2, 3 or 4 depending on the timepoint). Median. k) Intracellular CG of the indicated IDLV features retrieved over time from HSPCs edited with the optimized one-or two-hits protocol (n=2). To account for the low but detectable signal coming from extracellular cell-associated viral DNA, cells edited in presence of heat inactivated IDLV were used as control for each condition and their CG values subtracted to the paired treatment samples (see Methods for further details). Median. Note that these experiments were performed in parallel with those in FIG. 10. I) Heatmaps representing CG measured by the indicated ddPCR probe systems in single colonies plated 4 d after AAVS1 editing with the indicated optimized IDLV and ssAAV protocols (n=48, 24, 48, 21, 52, 32). Edited HSPCs were FACS-sorted for GFP expression before plating. To account for the residual presence of episomal DNA and increase stringency of the analysis, a lower threshold was set at 0.5 CG and signals<0.5 assigned zero in the heatmap (white cells). m) Heatmaps as in FIG. 71 showing CG in colonies plated 4 d after B2M editing and transduction with IDLV or ssAAV bearing the unrelated AAVS1 repair template (n=16, 44, 15, 40, 16, 43). n) CG measured by ddPCR probing for an AAVS1 sequence proximal (telomeric) to the left HA (807 bp apart from the sgRNA cut site) (n=7, 48, 23, 8, 52, 32). Mean. The dashed black line indicates the expected number of CG for diploid cells. The red lines indicate the cut-off values for colonies carrying long-range deletions (red and orange dots). The orange dot corresponds to the colony highlighted in FIG. 9f with AURKC CG<1.5. 0-p) Percentage of circulating hCD45⁺ (o) and GFP⁺ cells within the human graft (p) in mice transplanted with the outgrown progeny of starting-matched saturating cell doses of mPB HSPCs edited at AAVS1 with indicated optimized treatments (n=5 mice/group). Mean±s.e.m., LME followed by post-hoc analysis where groups have been compared at fixed timepoints. Results are shown for the last timepoint. q) CG measured by HIV and ITR ddPCR probes within human BM cells from mice in FIG. 9g, h (n=5 mice/group). Median with quartiles. Kruskal-Wallis test with Dunn's multiple comparisons. r) Percentage of AAVS1 alleles within human splenocytes from FIG. 9g, h carrying ITR or LTR DNA fragments (length≥20 bp) (n=5 mice/group). Median with quartiles. Kruskal-Wallis test with Dunn's multiple comparisons. s) Pie charts showing the proportion of fragments aligning to IDLV (left, n=33 alleles distributed across 5 mice) and AAV (right, n=48 alleles distributed across 12 mice) genomic features from experiment in FIG. 9g, h. t) Heatmap as in FIG. 71 representing the CG measured by the indicated ddPCR probes in colonies plated from FACS-sorted GFP⁺ CD34⁺ HSPCs harvested from BM of hematochimeric mice 14 weeks after transplant of human mPB HSPCs edited with the optimized AAV protocol (n=7). For all panels, “n” indicates biologically independent experiments or animals.

FIG. 8. Retrieval of IDLV IS in edited LT-HSCs. a-b) Percentage of circulating hCD45⁺ (a) and GFP⁺ cells within the human graft (b) of mice transplanted with the outgrown progeny of starting-matched limiting cell doses of CB HSPCs edited at AAVS1 with indicated treatments (n=10, 6, 11). Mean±s.e.m., LME followed by post-hoc analysis where groups have been compared at fixed timepoints. Results are shown for the last timepoint. c-d) Percentage of hCD45⁺ (c) and GFP⁺ cells within the human graft (d) in BM of transplanted mice from FIG. 8a, b (n=10, 6, 11). Mean±s.e.m., LME. Statistics are shown for the last timepoint. e-f) Percentage of hCD45⁺ (e) and GFP⁺ cells within the human graft (f) in bone marrow of mice transplanted with CB HSPCs edited in AAVS1 with an IDLV-based protocol (n=4). Median. g) Percentage of IDLV IS represented by the indicated number of genomes from mice in FIG. 8e, f (n=4). Mean±s.e.m. h) Genome-wide distribution of IDLV integrations retrieved from mice in FIG. 8e, f. The vast majority of IDLV integrations were detected at the LS sgRNA on-target site and its major off-target site (LAMC3). i) Enlarged genomic view as in FIG. 5d of IDLV IS in AAVS1 (top) and in two predicted off-target sites, LAMC3 (middle) and ZDHH8 (bottom). For comparison AAV IS in the same loci were also reported. j). Enlarged genomic view as in FIG. 6f of IDLV IS within the PGK gene. For comparison AAV IS in the same locus were also reported.

FIG. 9. Improved editing efficiency and mitigated cyto- and geno-toxicity by optimized IDLV template delivery. a) Percentage of GFP⁺ cells within mPB HSPC subpopulations 4 d after AAVS1 editing with different IDLV doses for transduction hit (n=1). b) HSPC culture composition 4 d after editing in experiments from FIG. 4g (n=1; UT: n=2). Mean. c) Bar plots showing the percentage of colonies from heatmaps in FIG. 41 positive for each event (3′TI±integration of viral features or WT/NHEJ-edited, left) and more in details, for each combination of ddPCR probe systems (right). The numbers of colonies analyzed for each condition are shown above the bars. d) Heatmaps representing the CG measured by the indicated ddPCR probes in single colonies plated 4 d after transduction with the indicated protocols (n=24, 28, 25). Edited HSPCs were plated as bulk population. To account for possible residual episomal DNA and increase stringency of the analysis, a lower threshold was set at 0.5 CG and signals<0.5 CG assigned zero in the heatmap (white cells). e) Bar plots showing the percentage of colonies from heatmaps in FIG. 4m positive for each event (3′TI±integration of viral features or WT/NHEJ-edited, left) as in FIG. 9c. f) CG measured by ddPCR probing for AURKC sequence about 2 Mbp telomeric to the AAVS1 sgRNA cut site (n=7, 21) in red and orange colonies from FIG. 4n (CFU) and healthy donor untreated cells (HD). Mean. The dashed black line indicates the expected number of CG for diploid cells. The orange lines indicate the cut-off values for colonies carrying long-range deletions (orange). g-h) Percentage of hCD45⁺ cells within live cells (g), GFP⁺ cells within hCD45⁺ cells (h), in organs of transplanted mice from FIG. 40, p (n=5 mice/group). Median with quartiles. i-I) Percentage of GFP⁺ cells within hCD45⁺ cells (i, j; median with quartiles) and percentage of cells for each hematopoietic lineage within hCD45⁺ cells (k, I; mean±s.e.m.) from spleen (i, k) or bone marrow (j, I) of transplanted mice from FIG. 40, p (n=5 mice/group). m) Number of CG measured by HIV and ITR ddPCR probes within human splenocytes from mice in FIG. 9f, g (n=5 mice/group). Median with quartiles. Kruskal-Wallis test with Dunn's multiple comparisons. n-o) Number (n) and percentage (o) of AAVS1 alleles in human splenocytes carrying integrated fragments (length≥20 bp) for each mouse (n=5 mice/group). Median with quartiles. Kruskal-Wallis test with Dunn's multiple comparisons. p) Bar plot showing the percentage of colonies from heatmaps in FIG. 4t positive for each event (3′TI±integration of viral features, left) and more in details, for each combination of ddPCR probe systems (right). The numbers of colonies analyzed for each condition are shown above the bars. q) Graphical representation of all possible HDR and/or NHEJ-mediated integration at both sides of the DNA DSB. For all panels, “n” indicates biologically independent experiments or animals.

FIG. 10. Schematics and details of AAV and IDLV constructs and primers/probes design for ddPCR analyses. a) Schematic representation of the ssAAV2/6 constructs carrying the transgene cassette and homology arms targeting AAVS1 (top, 1), CD40L (middle, 2) or IL2RG (bottom, 3). b) Sequences of the AAV ITR-2 (blue box) and AAV ITR-5 (green box). The different ITR portions are indicated: D, A/A′, B/B′, and C/C′ sequences, the rep-binding element (RBE) and the terminal resolution site (trs). Divergent nucleotides between the two sequences are shown in red. Putative p53 binding sites identified by the Alggen-Promo web tool with dissimilarity margin≤10% (http://alggen.Isi.upc.es/cgi-bin/promo_v3/promo/promoinit.cgi?dirDB=TF_8.3) are underlined in orange. c) Schematic representation of the scAAV2/6 construct carrying the transgene cassette and homology arms targeting AAVS1 (4). d) Sedimentation distribution plots from analytical ultracentrifugation (AUC) representing sedimentation coefficients “S” with interference fringes “J” (left) or absorbance “Abs” (right). AUC has been performed with AAVS1 ssAAV2/6 vector (FIG. 10a, top 1) fractionated by CsCl gradient with “empty” (top) and “full” (middle) particles or entirely AVB purified (bottom). The relative percentages of each capsid species are: 79.4% (empty˜ 63S), 12.7% (intermediate ˜ 76S), and 7.94% (full ˜ 93S) for the “empty” fraction; 100% (full ˜ 91S) for the “full” fraction, harboring the full-length (˜ 3,000-nucleotides) vector genome. AVB fraction is composed of mixed AAV particles with 23.2% (empty˜ 60S) and 69.8% (full ˜ 89S). The remaining fraction 7% is considered as non-determined (>110S). e) Experimental workflow for ex vivo HDR gene editing using ssAAV2/6 or IDLV as donor template. f) Panel of ddPCR probes tiling the AAVS1 AAV2/6 vector genome (“ITR”, “ITR+cargo”, “GFP”), the 3′AAVS1 vector-genome junction (referred to as “3′TI” or “3′HDR”) and the neighboring loci (“extHA-L”, “AURKC”). g) Panel of ddPCR probes tiling the AAVS1 IDLV vector genome (“HIV”, “GFP”), the 3′AAVS1 vector-genome junction (“3′TI” or “3′HDR”) and the neighboring loci (“extHA-L”, “AURKC”). h) Primers (violet) and probes (light green) binding sites aligned on the reference plasmid sequence for AAVS1 ssAAV2/6. i) Primers (violet) and probes (light green) binding sites aligned on the reference plasmid sequence for AAVS1 scAAV2/6.

FIG. 11. Gating strategies for flow cytometry analyses. Gating strategies for the analysis of: a) HSPC phenotype 4 d after treatments; b) Human PBMCs or splenocytes in transplanted mice; c) Human cells within bone marrow of transplanted mice.

FIG. 12. Experimental workflow for ex vivo HDR gene editing using IDLV as donor template.

FIG. 13. Improved editing efficiency by optimized IDLV template delivery. A) Percentage of GFP⁺ cells within mPB HSPC subpopulations 4 d after AAVS1 editing with different possible transduction enhancers and different combinations as indicated. Cells were transduced with 1 hit of IDLV with indicated MOI at 24 hours pre-RNP electroporation (n=3 biological replicates). Mean. B) Percentage of GFP⁺ cells within mPB HSPC subpopulations 4 d after AAVS1 editing with different possible transduction enhancers and different combinations as indicated. Cells were transduced with 1 or 2 hit(s) of IDLV (MOI=150) at 24 or 12 hours pre-RNP electroporation (first hit) and +15 min post-RNP electroporation (second hit). dmPGE-2 was also tested as possible transduction enhancer. High-density (HD) cell concentration (4E6 HSPC/ml) was tested again standard (1E6 HSPC/ml) (n=1 biological replicate). C) Percentage of GFP⁺ cells within mPB HSPC subpopulations 4 d after AAVS1 editing with cells were transduced with 1 or 2 hit(s) of IDLV with indicated MOI at 12 hours pre-RNP electroporation (first hit) and +/−15 min post-RNP electroporation (second hit). (n=1 biological replicate).

FIG. 14. Improved editing efficiency by optimized IDLV template delivery. A) Experimental workflow for ex vivo HDR gene editing using ssAAV2/6 or IDLV as donor template. B) Percentage of GFP⁺ cells within mPB HSPC subpopulations 4 d after AAVS1 editing using IDLV for template delivery (“RNP+IDLV”) in presence or absence of CsH and GSE56/E4orf6/7. Transduction performed once 24 or 12 hours before (1 hit) without or with a second one immediately after (2 hits) electroporation, using multiplicity of infection (MOI) of 150 transducing units (TU^{293T})/cell per hit. Editing with the optimized ssAAV protocol (Ferrari et al. 2020) performed in parallel (n=1, 2, 2, 5, 4, 5). Median. C) Number of colonies grown from seeded mPB HSPCs edited with the indicated optimized treatments (n=6). Median with 95% Cl. Friedman test with Dunn's multiple comparisons. D) Fold change expression of CDKN1A over time relative to UT after AAVS1 editing with IDLV or AAV optimized protocols in mPB HSPCs (n=8/6/6, 8/6/6 or 8/4/4 including biological and technical replicates). Median. Kruskal-Wallis test with Dunn's multiple comparisons at the last time point. E) Quantification of NBS1 positive cells and number of foci over time from HSPCs edited with the indicated treatments (n=5, 3, 5, 2, 5). Mean. F-G) Percentage of circulating hCD45⁺ (F) and GFP⁺ cells within the human graft (G) in mice transplanted with the outgrown progeny of starting-matched saturating cell doses of mPB HSPCs edited at AAVS1 with indicated optimized treatments (n=5 mice/group). Mean±/−s.e.m., LME followed by post-hoc analysis where groups have been compared at fixed timepoints. Results are shown for the last timepoint.

FIG. 15. A) Schematic representation of the corrective donor cassette. B) Gene editing procedure in mPB CD34⁺ cells exploiting the IDLV targeting vector and AAV6 vector. IDLV vectors were tested at different doses, timing and rounds. C) Percentage of edited alleles in bulk HD mPB-HSPCs using the selected GE conditions. D) Percentage of edited alleles in HSPCs subpopulations. E) Composition of hematopoietic stem cell. F) Clonogenic potential of HD edited HSPCs (comparisons AAV6 vs IDLV).

FIG. 16. HDR-mediated gene editing in human HSPCs by combining IDLV and lipid nanoparticles (LNPs). A) Percentage of GFP⁺ cells, as surrogate marker of cells bearing HDR editing, within HSPC subpopulations 96 hours after treatments. UT=untreated. B) Culture composition of HDR edited HSPCs. The gene editing treatment is not impacting the HSPC phenotype ex vivo.

### DETAILED DESCRIPTION OF THE INVENTION

The terms “comprising”, “comprises” and “comprised of′ as used herein are synonymous with “including” or “includes”; or “containing” or “contains”, and are inclusive or open-ended and do not exclude additional, non-recited members, elements or steps. The terms “comprising”, “comprises” and “comprised of′ also include the term “consisting of”.

Numeric ranges are inclusive of the numbers defining the range.

### Uses

In one aspect, the present invention provides for use of a combination of (a) cyclosporin H (CsH) or a derivative thereof, and (b) a p53 inhibitor and/or an adenoviral protein, or one or more nucleotide sequences encoding therefor, for increasing the efficiency of gene editing of an isolated population of cells when transduced by a viral vector.

In one aspect, the present invention provides for use of a combination of (a) CsH or a derivative thereof, and (b) a p53 inhibitor and/or an adenoviral protein, or one or more nucleotide sequences encoding therefor, for increasing the efficiency of transduction of an isolated population of cells by a viral vector.

In one aspect, the present invention provides for use of a combination of (a) CsH or a derivative thereof, and (b) a p53 inhibitor and/or an adenoviral protein, or one or more nucleotide sequences encoding therefor, for increasing the survival and/or engraftment of gene-edited cells.

In one embodiment, the combination comprises the cyclosporin H (CsH) or a derivative thereof and the p53 inhibitor. In one embodiment, the combination comprises the cyclosporin H (CsH) or a derivative thereof and the adenoviral protein. In one embodiment, the combination comprises the cyclosporin H (CsH) or a derivative thereof, the p53 inhibitor and the adenoviral protein.

Optionally, the combination of CsH or a derivative thereof, and p53 inhibitor and/or an adenoviral protein, is used in combination with at least one additional transduction enhancer.

In one embodiment, the CsH or a derivative thereof is contacted with the cells simultaneously, sequentially or separately in combination with the p53 inhibitor and/or adenoviral protein.

In one embodiment, the p53 inhibitor and/or adenoviral protein is contacted with the cells simultaneously, sequentially or separately in combination with the CsH or a derivative thereof.

### Methods of Transducing a Population of Cells

In one aspect, the present invention provides a method of transducing a population of cells comprising the step of contacting the population of cells with a combination of (a) CsH or a derivative thereof, and (b) a p53 inhibitor and/or an adenoviral protein, or one or more nucleotide sequences encoding therefor. Suitably, the method further comprises transducing the population of cells with a viral vector.

In one aspect, the present invention provides a method of transducing a population of cells comprising the steps of:
i) contacting the population of cells with a combination of (a) CsH or a derivative thereof, and (b) a p53 inhibitor and/or an adenoviral protein, or one or more nucleotide sequences encoding therefor; and
ii) transducing the population of cells with a viral vector.

In one aspect, the present invention provides a method of transducing a population of cells comprising the steps of:
i) contacting the population of cells with a combination of (a) CsH or a derivative thereof, and (b) a p53 inhibitor and/or an adenoviral protein, or one or more nucleotide sequences encoding therefor; and
ii) transducing the population of cells with a viral vector; and
iii) introducing gene-editing machinery, such as a site-directed nuclease, to the cells.

The method of transduction may increase the efficiency of transduction of the population of cells by the viral vector and/or increase the efficiency of gene editing of the population of cells when transduced by the viral vector.

The method of the present invention may increase the survival and/or engraftment of gene-edited cells.

In one embodiment, the method substantially prevents or reduces apoptosis in the cells, in particular during in vitro culture.

In one embodiment, at least 1%, 2%, 3%, 4%, 5%, 6%, 7%, 8%, 9%, 10%, 15%, 20%, 25%, 30%, 40%, 50% or 75%, preferably at least 25%, fewer cells become apoptotic following culture for a period of time (e.g. about 6 or 12 hours, or 1, 2, 3, 4, 5, 6, 7 or more days, preferably about 2 days) when the cells have been exposed to the combination rather than in its absence. Preferably, the period of time begins with the transduction of the cells with a viral vector.

In one embodiment, at least 1%, 2%, 3%, 4%, 5%, 6%, 7%, 8%, 9%, 10%, 15%, 20%, 25%, 30%, 40%, 50% or 75%, preferably at least 10%, more transplanted cells engraft in a host subject when the cells have been exposed to the combination rather than in its absence.

In some embodiments, the present invention provides a method of transducing a population of cells comprising the steps of:
i) contacting the population of cells with a combination of (a) CsH or a derivative thereof, and (b) a p53 inhibitor and/or an adenoviral protein, or one or more nucleotide sequences encoding therefor;
ii) transducing the population of cells with a viral vector; and
iii) optionally, introducing gene-editing machinery to the cells such that the efficiency of transduction of the population of cells by the viral vector and/or the efficiency of gene editing of the population of cells when transduced by the viral vector and/or the survival of gene-edited cells and/or engraftment of gene-edited cells is increased.

In some embodiments, the method steps are carried out ex vivo or in vitro.

Optionally, the population of cells is contacted with a least one additional transduction enhancer (e.g. lentiBOOST or a PGE2, such as dmPGE2).

The cells may be contacted with the agents (e.g. CsH or a derivative thereof, a p53 inhibitor and/or an adenoviral protein) simultaneously, sequentially or separately. The term “simultaneous” as used herein means that the agents are used concurrently, i.e. at the same time. The term “sequential” as used herein means that the agents are used one after the other. The term “separate” as used herein means that the agents are used independently of each other but within a time interval that allows the agents to show a combined, preferably synergistic, effect. Thus, using “separately” may permit one agent to be used, for example, within 1 minute, 5 minutes, 10 minutes, 30 minutes or one hour after the other.

The population of cells may be contacted with the agents before, before and during, during, during and after, or after contact with the viral vector, or combinations thereof.

In one embodiment, the population of cells is contacted with CsH or a derivative thereof before and/or during contact with the viral vector. In one embodiment, the population of cells is contacted with CsH or a derivative thereof before and during contact with the viral vector.

The population of cells may be contacted with the agents for any suitable period of time. In one embodiment, the population of cells is contacted with CsH or a derivative thereof for at least about 30 minutes, at least about 1 hour, or at least about 2 hours before transduction and/or at least about 8 hours, at least about 12 hours, at least about 16 hours, at least about 20 hours, or at least about 24 hours, e.g. during transduction. In one embodiment, the population of cells is contacted with CsH or a derivative thereof for about 1 hour to about 6 hours, or about 2 hours to about 4 hours before transduction and/or about 12 hours to about 24 hours, or about 16 hours to about 20 hours, e.g. during transduction.

In some embodiments, the population of cells is stimulated before and/or during the method of the invention. In one embodiment, the population of cells is stimulated before transduction. In one embodiment, the population of cells is stimulated during transduction.

Quiescent cells (e.g. quiescent HSPCs) typically require extensive cytokine-mediated stimulation for efficient transduction (Zielske, S. P. and Gerson, S. L., 2003. Molecular Therapy, 7 (3), pp. 325-333). Cytokines for stimulating quiescent cells (e.g. quiescent HSPCs) are known to those of skill in the art and include, for example early-acting cytokines such as IL-3, IL-6, stem cell factor (SCF), and Fit-3L. In one embodiment, the population of cells is contacted with cytokines (e.g. early-acting cytokines) before and/or during transduction. In one embodiment, the population of cells is contacted with recombinant human stem cell factor (rhSCF), recombinant human thrombopoietin (rhTPO), recombinant human Flt3 ligand (rhFlt3), or recombinant human IL6 (rhIL6) before and/or during transduction.

In one embodiment, the population of cells is contacted with cytokines before transduction.

In one embodiment, the population of cells is contacted with additional agents, such as expansion enhancers, before and/or during transduction.

As used herein, an “expansion enhancer” is a substance that is capable of improving expansion of cells (e.g. HSCs, HPCS, and/or LPCs or CD34⁺ cells). Suitable expansion enhancers include UM171, UM729, StemRegenin1 (SR1), diethylaminobenzaldehyde (DEAB), LG1506, BIO (GSK3ß inhibitor), NR-101, trichostatin A (TSA), garcinol (GAR), valproic acid (VPA), copper chelator, tetraethylenepentamine, and nicotinamide. In some embodiments, the stimulation is carried out in the presence of at least one expansion enhancer. Any suitable concentration of expansion enhancer may be used, for example as described in Huang, X., et al., 2019. F1000Research, 8, 1833.

In some embodiments, the stimulation is carried out in the presence of UM171 or UM729. The concentration of UM171 may be about 10-200 nM, about 20-100 nM, or about 50 nM. In one embodiment, the concentration is about 35 nm.

In some embodiments, the stimulation is carried out in the presence of SR1. The concentration of SR1 may be about 0.1-10 μM, about 0.5-5 μM, or about 1 μM. In one embodiment, the concentration is about 1 μM.

In some embodiments, the stimulation is carried out in the presence of UM171 (e.g. in a concentration of about 35 nM) and SR1 (e.g. in a concentration of about 1 μM).

In some embodiments, the stimulation is carried out in the presence of SCF (e.g. in a concentration of about 300 ng/ml), FLT3-L (e.g. in a concentration of about 300 ng/ml), TPO (e.g. in a concentration of about 100 ng/ml), PGE2 (e.g. in a concentration of about 10 μM), UM171 (e.g. in a concentration of about 35 nM), and SR1 (e.g. in a concentration of about 1 μM).

In some embodiments, the population of cells is stimulated before transduction, such as for a period of 1 to 5 days, such as 2 to 4 days, such as 2 to 3 days before transduction. In one embodiment, the population of cells is stimulated for 2 days before transduction. In one embodiment, the population of cells is stimulated for 2.5 days before transduction.

The gene-editing machinery, such as a site-directed nuclease, may be introduced to the cells using any suitable method, for example, by electroporation.

In some embodiments, contact of the cells with the gene-editing machinery (e.g. site-directed nuclease) occurs about 4 to 48 hours after contact with the viral vector (e.g. the beginning of the transduction step), such as about 6 to 36 hours, 8 to 24 hours, or 10 to 14 hours. In some embodiments, contact of the cells with the gene-editing machinery (e.g. site-directed nuclease) occurs about 12 hours after the beginning of the transduction step. In some embodiments, contact of the cells with the gene-editing machinery (e.g. site-directed nuclease) occurs about 24 hours after the beginning of the transduction step.

In some embodiments, contact of the cells with the gene-editing machinery (e.g. site-directed nuclease) occurs about 10 to 30 minutes before contact with the viral vector (e.g. the beginning of the transduction step), such as about 10 to 25 minutes, or 10 to 20 minutes. In some embodiments, contact of the cells with the gene-editing machinery (e.g. site-directed nuclease) occurs about 15 minutes before the beginning of the transduction step.

In one embodiment, the population of cells is contacted with a p53 inhibitor and/or an adenoviral protein before and/or during contact with the site-directed nuclease. In one embodiment, the population of cells is contacted with a p53 inhibitor and/or an adenoviral protein before and during contact with the site-directed nuclease. In one embodiment, the population of cells is contacted with a p53 inhibitor and/or an adenoviral protein during contact with the site-directed nuclease.

Thus, in one embodiment, the present invention provides a method of transducing a population of cells comprising the steps of:
i) contacting the population of cells with cyclosporin H (CsH) or a derivative thereof,
ii) transducing the population of cells with a viral vector;
iii) contacting the population of cells with a p53 inhibitor and/or an adenoviral protein, or one or more nucleotide sequences encoding therefor; and
iv) introducing a site-directed nuclease to the cells.

Suitably, the method steps are carried out in the order listed.

The population of cells may be transduced by the viral vector in a single transduction step (“one-hit”) or in two transduction steps (“two-hit”).

In one embodiment, the present invention provides a method of transducing a population of cells comprising the steps of:
i) contacting the population of cells with cyclosporin H (CsH) or a derivative thereof,
ii) transducing the population of cells with a viral vector;
iii) contacting the population of cells with a p53 inhibitor and/or an adenoviral protein, or one or more nucleotide sequences encoding therefor;
iv) introducing a site-directed nuclease to the cells; and
v) transducing the population of cells with a viral vector.

Suitably, the method steps are carried out in the order listed.

In some embodiments, the second transduction step occurs immediately after contacting the cells with a site-directed nuclease. In one embodiment, the second transduction step occurs about 15 minutes after contacting the cells with a site-directed nuclease.

In some embodiments, the cells are transduced with viral vector at an MOI of between around 10 to 250, such as 50 to 250, such as 100 to 200. In some embodiments, the cells are transduced with viral vector at an MOI of 10, 20, 30, 40, 50, 60, 70, 80, 90, 100, 110, 120, 130, 140, 150, 160, 170, 180, 190, 200, 210, 220, 230, 240, or 250. In some embodiments, the cells are transduced with viral vector at an MOI of 20. In some embodiments, the cells are transduced with viral vector at an MOI of 100. In some embodiments, the cells are transduced with viral vector at an MOI of 150. In some embodiments, the cells are transduced with viral vector at an MOI of 200.

In some embodiments, the cells are seeded at a concentration of between around 1×10⁵ to 1×10⁶ cells per ml, such as a 1×10⁵ to 5×10⁵cells per ml. In some embodiments, the cells are seeded at a concentration of around 5×10⁵ cells per ml.

### Other Methods of Enhancing Transduction

The use or method of the present invention may be combined with any other suitable methods to further increase the efficiency of transduction and/or gene editing.

Exemplary methods for further increasing the efficiency of transduction and/or gene editing include: contacting the population of cells with one or more additional transduction enhancer; high density culture; viral capsid mutants; alternative Env glycoproteins or VSV-g fusions; pre-stimulation in the presence of cytokines or HSC expansion; combining methods to enhance virus-cell interaction, and spinoculation.

In one embodiment, the use or method further comprises one or more of: contacting the population of cells with one or more additional transduction enhancer; high density culture; viral capsid mutants; alternative Env glycoproteins or VSV-g fusions; pre-stimulation in the presence of cytokines or HSC expansion; combining methods to enhance virus-cell interaction; and spinoculation.

In one embodiment, as described above, the population of cells may be further contacted with one or more additional transduction enhancers. The cells may be contacted with one or more additional transduction enhancers at any point prior to or during transduction, for example at the same time as the CsH or a derivative thereof. In one embodiment, the method comprises contacting the population of cells with one or more additional transduction enhancers before and/or during transduction. In one embodiment, the method comprises contacting the population of cells with one or more additional transduction enhancers simultaneously, sequentially or separately with the CsH or a derivative thereof.

In one embodiment, transduction takes place in a high-density culture. High density culture conditions may include a cell density of about 1e6 cells/mL or greater (e.g. 1e6 to 4e6 cells/mL). For example, Uchida N, et al. (2019) Mol Ther Methods Clin Dev 13:187-196 describes high-efficiency lentiviral transduction of human CD34 (+) cells in high-density culture (4e6/mL). In one embodiment, the population of cells is transduced at a cell density of about 1e6 cells/mL or greater or at a cell density of about 1e6 to about 4e6 cells/mL.

In one embodiment, the viral vector comprises viral capsid mutants which increase the efficiency of transduction and/or gene editing. Such viral capsid mutants will be known to those of skill in the art. For example, suitable lentiviral CA mutants are described in Petrillo C, et al (2015) Mol Ther 23:352-362, including the A88T CA mutant.

In one embodiment, the viral vector comprises alternative Env glycoproteins and/or VSV-g fusions which increase the efficiency of transduction and/or gene editing. Such Env glycoproteins or VSV-g fusions will be known to those of skill in the art. For example, Hanawa H, et al (2002) Mol Ther 5:242-251 describes a comparison of various envelope proteins for their ability to pseudotype lentiviral vectors and transduce primitive hematopoietic cells from human blood.

In one embodiment, the population of cells are pre-stimulated in the presence of cytokines and/or the use or method comprises HSC expansion. Suitable conditions will be known to those of skill in the art. For example, Uchida N, et al (2011) Gene Ther 18:1078-1086 describes optimal conditions for lentiviral transduction of engrafting human CD34⁺ cells.

In one embodiment, the use or method comprises a combining method to enhance virus-cell interaction. Suitable conditions will be well known to those of skill in the art. For example, suitable conditions are described in Liu H, et al (2000) Leukemia 14:307-311.

In one embodiment, the use or method comprises spinoculation. Spinoculation may enhance contact between viral particles and target cells. Suitable conditions will be well known to those of skill in the art. For example, suitable conditions are described in Millington M, et al (2009) PLOS One 4: e6461.

### Increasing the Efficiency of Transduction

Increasing the efficiency of transduction refers to an increase in the transduction of the cells in the presence of the combination, in comparison to the transduction achieved in the absence of the combination but under otherwise substantially identical conditions. An increased efficiency of transduction may therefore allow the multiplicity of infection (MOI) and/or the transduction time required to achieve effective transduction to be reduced.

In one embodiment, the percentage of cells transduced by the vector is increased. The percentage of cells transduced by the vector may be, for example, increased by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

In another embodiment, the vector copy number per cell is increased. The vector copy number per cell may be, for example, increased by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

Preferably, both are achieved at the same time.

Methods for determining the percentage of cells transduced by a vector are known in the art. Suitable methods include flow cytometry, fluorescence-activated cell sorting (FACS) and fluorescence microscopy. The technique employed is preferably one which is amenable to automation and/or high throughput screening.

For example, a population of cells may be transduced with a vector which harbours a reporter gene. The vector may be constructed such that the reporter gene is expressed when the vector transduces a cell. Suitable reporter genes include genes encoding fluorescent proteins, for example green, yellow, cherry, cyan or orange fluorescent proteins. Once the population of cells has been transduced by the vector, both the number of cells expressing and not-expressing the reporter gene may be quantified using a suitable technique, such as FACS. The percentage of cells transduced by the vector may then be calculated.

Alternatively, quantitative PCR (qPCR) may be used to determine the percentage of cells transduced by a vector that does not harbour a reporter gene. For example, single colonies of cells (e.g. CD34⁺ cells) may be picked from a semi-solid culture and qPCR may be performed on each colony separately to determine the percentage of vector-positive colonies among those analysed.

Methods for determining vector copy number are also known in the art. The technique employed is preferably one which is amenable to automation and/or high throughput screening. Suitable techniques include quantitative PCR (qPCR) and Southern blot-based approaches.

### Increasing the Efficiency of Gene Editing

Increasing the efficiency of gene editing may refer to an increase in the number of cells in which a target gene or site has been edited (e.g. disrupted, replaced, deleted or had a nucleic acid sequence inserted within or at it) in the intended manner following transduction of a population of cells with a viral vector in the presence of the combination, in comparison to that achieved in the absence of the combination but under otherwise substantially identical conditions. An increased efficiency of gene editing may therefore allow the multiplicity of infection (MOI) and/or the transduction time required to achieve effective gene editing to be reduced. Methods for determining whether a target gene or site has been edited are known in the art.

In the context of gene editing, for example using a CRISPR/Cas system, preferably the vector used to transduce the population of cells is a non-integrating vector (e.g. an integration-defective lentiviral vector, IDLV).

In one embodiment, the combination for use according to the present invention improves gene editing efficiency compared with gene editing without use of the combination (i.e. standard gene editing). Suitably, gene editing efficiency may be improved by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

Suitably, the gene editing efficiency may be improved in a particular cell compartment. Suitably, gene editing is improved in a primitive HSPC cell compartment. Suitably, gene editing may be improved in CD34⁺ CD133-cells. Suitably, gene editing may be improved in CD34⁺ CD133⁺ cells. Suitably, gene editing may be improved in CD34⁺ CD133⁺ CD90⁺ cells.

Preferably gene editing efficiency of CD34⁺ CD133⁺ CD90⁺ cells may be improved by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

### Cell Survival and Engraftment

The term “survival” refers to the ability of the cells to remain alive (e.g. not die or become apoptotic) during in vitro or ex vivo culture. Cells may, for example, undergo increased apoptosis following transduction with a viral vector during cell culture; thus, the surviving cells may have avoided apoptosis and/or cell death.

Cell survival may be readily analysed by the skilled person. For example, the numbers of live, dead and/or apoptotic cells in a cell culture may be quantified at the beginning of culture and/or following culture for a period of time (e.g. about 6 or 12 hours, or 1, 2, 3, 4, 5, 6, 7 or more days; preferably, the period of time begins with the transduction of the cells with a viral vector). The effect of the combination on cell survival may be assessed by comparing the numbers and/or percentages of live, dead and/or apoptotic cells at the beginning and/or end of the culture period between experiments carried out in the presence and absence of the combination, but under otherwise substantially identical conditions.

Cell numbers and/or percentages in certain states (e.g. live, dead or apoptotic cells) may be quantified using any of a number of methods known in the art, including use of haemocytometers, automated cell counters, flow cytometers and fluorescence activated cell sorting machines. These techniques may enable distinguishing between live, dead and/or apoptotic cells. In addition or in the alternative, apoptotic cells may be detected using readily available apoptosis assays (e.g. assays based on the detection of phosphatidylserine (PS) on the cell membrane surface, such as through use of Annexin V, which binds to exposed PS; apoptotic cells may be quantified through use of fluorescently-labelled Annexin V), which may be used to complement other techniques.

The term “engraftment” refers to the ability of the cells (e.g. haematopoietic stem and/or progenitor cells) to populate and survive in a subject following their transplantation, i.e. in the short and/or long term after transplantation. For example, engraftment may refer to the number and/or percentages of haematopoietic cells descended from the transplanted haematopoietic stem cells (e.g. graft-derived cells) that are detected about 1 day to 24 weeks, 1 day to 10 weeks, or 1-30 days or 10-30 days after transplantation. In the xenograft model of human haematopoietic stem and/or progenitor cell engraftment and repopulation, engraftment may be evaluated in the peripheral blood as the percentage of cells deriving from the human xenograft (e.g. positive for the CD45 surface marker), for example. In one embodiment, engraftment is assessed at about 1, 2, 3, 4, 5, 6, 7, 8, 9, 10, 15, 20, 25 or 30 days after transplantation. In another embodiment, engraftment is assessed at about 4, 5, 6, 7, 8, 9, 10, 11, 12, 13, 14, 15, 16, 17, 18, 19, 20, 21, 22, 23 or 24 weeks after transplantation. In another embodiment, engraftment is assessed at about 16-24 weeks, preferably 20 weeks, after transplantation.

Engraftment may be readily analysed by the skilled person. For example, the transplanted haematopoietic stem and/or progenitor cells may be engineered to comprise a marker (e.g. a reporter protein, such as a fluorescent protein), which can be used to quantify the graft-derived cells. Samples for analysis may be extracted from relevant tissues and analysed ex vivo (e.g. using flow cytometry).

Suitably, the combination for use according to the present invention may improve engraftment of gene edited haematopoietic stem and/or progenitor cells compared with gene editing without use of the combination. Suitably, engraftment at a given time point may be increased by at least 5%, 10%, 20%, 30%, 40%, 50%, 60%, 70%, 80%, 90% or more when compared with engraftment of untreated gene edited haematopoietic stem and/or progenitor cells.

In a preferred embodiment, an combination for use according to the invention does not adversely affect the growth of gene edited haematopoietic cells, haematopoietic stem cells and/or haematopoietic progenitor cells when compared with untreated gene edited haematopoietic cells, haematopoietic stem cells and/or haematopoietic progenitor cells.

### Combination

The present invention provides a combination of (a) cyclosporin H (CsH) or a derivative thereof, and (b) a p53 inhibitor and/or an adenoviral protein, or one or more nucleotide sequences encoding therefor, for use in a method of gene editing.

In some embodiments, the invention provides a combination of cyclosporin H (CsH) or a derivative thereof, a p53 inhibitor and an adenoviral protein, or one or more nucleotide sequences encoding therefor, for use in a method of gene editing.

The combination may be provided in any form, for example the cyclosporin H (CsH) or a derivative thereof, and p53 inhibitor and/or an adenoviral protein may be combined in a composition, in a kit-of-parts, and or applied in combination.

The combination may be any combination suitable for cell culture, e.g. the combination may be applied to a population of cells before, during, before and during, and/or after cell culture or contact with a viral vector, or any combination thereof. In one embodiment, the combination may be applied before and/or during cell culture. In one embodiment, the combination is suitable for cell transduction e.g. the combination may be applied to a population of cells before, during, before and during, or after contact with a viral vector, or any combination thereof. In one embodiment, the combination may be applied before and/or during contact with a viral vector. The agents may be, for example, applied to the population of cells simultaneously, sequentially or separately. In one embodiment, the combination is for improving the transduction of cells by viral vectors and/or improving gene editing of cells.

### Cyclosporin H

Cyclosporin H (CsH, CAS No. 83602-39-5) is a cyclic undecapeptide having the following structure:

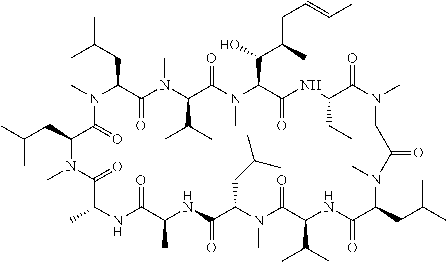

CsH is known to selectively antagonise the formyl peptide receptor, however unlike cyclosporin A (CsA), CsH does not bind cyclophilin to evoke immunosuppression. CsA mediates immunosuppression as a complex with the host peptidyl-prolyl isomerase cyclophilin A (CypA). This inhibits the Ca²⁺-dependent phosphatase calcineurin and consequent activation of pro-inflammatory cytokines such as IL-2 (Sokolskaja, E. et al. (2006) Curr. Opin. Microbiol. 9:404-8).

Solutions of CsH for use in the present invention may be prepared using routine methods known in the art.

The present invention encompasses the use of CsH and derivatives of CsH. The CsH derivatives of the present invention are those which increase the efficiency of transduction of an isolated population of cells by a viral vector and/or increasing the efficiency of gene editing of an isolated population of cells when transduced by a viral vector.

CsH derivatives of the present invention may have been developed for increased solubility, increased stability and/or reduced toxicity.

CsH derivatives of the invention are preferably of low toxicity for mammals, in particular humans. Preferably, CsH derivatives of the invention are of low toxicity for haematopoietic stem and/or progenitor cells; and/or T cells.

Suitable CsH derivatives may be identified using methods known in the art for determining transduction efficiency and/or gene editing. For example, methods for determining the percentage of cells that are transduced by a vector, or methods for determining the vector copy number per cell may be employed. Such methods have been described above. The method employed is preferably one which is amenable to automation and/or high throughput screening of candidate CsH derivatives. The candidate CsH derivatives may form part of a library of CsH derivatives.

The concentration at which CsH or a derivative thereof is applied to a population of cells may be adjusted for different vector systems to optimise transduction efficiency and/or gene editing. Methods for determining transduction efficiency and gene editing have been described above. A skilled person may therefore select a suitable concentration of CsH or a derivative thereof to maximise increase in transduction efficiency and/or gene editing while minimising any toxicity using the approaches described herein.

In one embodiment, the CsH or derivative thereof is at a concentration of about 1-50 μM. In another embodiment, the CsH or derivative thereof is at a concentration of about 5-50 μM. In another embodiment, the CsH or derivative thereof is at a concentration of about 10-50 μM.

In another embodiment, the CsH or derivative thereof is at a concentration of about 1-40, 5-40 or 10-40 μM. In another embodiment, the CsH or derivative thereof is at a concentration of about 1-30, 5-30 or 10-30 μM. In another embodiment, the CsH or derivative thereof is at a concentration of about 1-20, 5-20 or 10-20 μM. In another embodiment, the CsH or derivative thereof is at a concentration of about 1-15, 5-15 or 10-15 μM.

In another embodiment, the CsH or derivative thereof is at a concentration of about 1-15, 2-14, 3-13, 4-12, 5-11, 6-10 or 7-9 μM. For example, the concentration of CsH may be about 1, 2, 3, 4, 5, 6, 7, 8, 9, 10, 11, 12, 13, 14, 15, 20, 25, 30, 35, 40, 45 or 50 μM. In a preferred embodiment, the concentration of CsH or a derivative thereof is about 5, 6, 7, 8, 9, 10, 11, 12, 13, 14 or 15 μM. In a particularly preferred embodiment, the concentration of CsH or a derivative thereof is about 8 μM.

p53 inhibitor

The term “p53 inhibitor” refers to an inhibitor of p53 activation, the terms may be used interchangeably.

In some embodiments, the p53 inhibitor directly inhibits p53.

The term “p53 activation” refers to an increase in the activity of p53, for example through a post-translational modification of the p53 protein. Example post-translational modifications include phosphorylation, acetylation and methylation, and are described in Kruse, J. P. et al. (2008) SnapShot: p53 Posttranslational Modifications Cell 133:930-931. In the context of the invention, the p53 activation preferably results from phosphorylation of p53, particularly preferably at amino acid Serine 15.

Methods for analysing such post-translational modifications are known in the art (example methods for analysing kinase activity are disclosed herein, further methods include, for example, mass spectrometry- and antibody recognition-based methods).

An example amino acid sequence of p53, which may be used to provide an amino acid numbering convention, is:

(SEQ ID NO: 1; NCBI Accession No. 000537.3)
MEEPQSDPSVEPPLSQETFSDLWKLLPENNVLSPLPSQAMDDLMLSPDDIEQWFTEDPGPDEAPRMPEAA
PPVAPAPAAPTPAAPAPAPSWPLSSSVPSQKTYQGSYGFRLGFLHSGTAKSVTCTYSPALNKMFCQLAKT
CPVQLWVDSTPPPGTRVRAMAIYKQSQHMTEVVRRCPHHERCSDSDGLAPPQHLIRVEGNLRVEYLDDRN
TFRHSVVVPYEPPEVGSDCTTIHYNYMCNSSCMGGMNRRPILTIITLEDSSGNLLGRNSFEVRVCACPGR
DRRTEEENLRKKGEPHHELPPGSTKRALPNNTSSSPQPKKKPLDGEYFTLQIRGRERFEMFRELNEALEL
KDAQAGKEPGGSRAHSSHLKSKKGQSTSRHKKLMFKTEGPDSD.

### Dominant Negative Peptides

In one embodiment, the inhibitor of p53 activation is a mutant p53 peptide.

In a preferred embodiment, the inhibitor of p53 activation is a dominant negative peptide (e.g. a dominant negative p53 peptide).

Suitably, a dominant negative peptide may comprise mutations in the homo-oligomerisation domain. Suitably, dominant negative peptides comprising mutations in the homo-oligomerisation domain may dimerise with wild-type p53 and prevent wild-type p53 from activating transcription.

In a preferred embodiment, the dominant negative peptide is GSE56 or a variant thereof.

In one embodiment, a nucleotide sequence encoding GSE56 is set forth in SEQ ID NO: 2.

(SEQ ID NO: 2)
ATGGATGGATGGTGTCCTGGGAGAGACCGTCGGACAGAGGAAGAAAATT
TCCGCAAAAAAGAAGAGCATTGCCCGGAGCTGCCCCCAGGGAGTGCAAA
GAGAGCACTGCCCACCAGCACAAGCTCCTCTCCCCAGCAAAAGAAAAAA
CCACTCGATGGAGAATATTTCACCCTTAAGATCCGTGGGCGTGAGCGCT
TCGAGATGTTCCGAGAGCTGAATGAGGCCTTGGAATTAAAGGATGCCCG
TGCTGCCGAGGAGTCAGGAGACAGCAGGGCTCACTCCAGCTACCCGAAG
ATAGTTAGTTAG

In one embodiment, the amino acid sequence of GSE56 is set forth in SEQ ID NO: 3.

(SEQ ID NO: 3)
MDGWCPGRDRRTEEENFRKKEEHCPELPPGSAKRALPTSTSSSPQQKKK
PLDGEYFTLKIRGRERFEMFRELNEALELKDARAAEESGDSRAHSSYPK
IVS

Suitably, the inhibitor of 53 activation may be a nucleotide sequence which encodes GSE56. Suitably, the inhibitor of p53 activation may be GSE56 polypeptide. Suitably, the inhibitor of p53 activation may be mRNA encoding GSE56.

In some embodiments, the p53 inhibitor comprises or consists of the sequence SEQ ID NO: 3 (or a polynucleotide encoding therefor), or comprises or consists of an amino acid sequence having at least 70%, 75%, 80%, 85%, 90%, 95%, 96%, 97%, 98%, or 99% sequence identity to SEQ ID NO: 3 (or a polynucleotide encoding therefor).

### Pifithrin-a

In one embodiment, the p53 inhibitor is pifithrin-a, pifithrin-a cyclic and pifithrin-a p-nitro or a derivative thereof. Pifithrin-a has the structure:

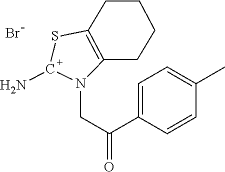 [SMILES: Br ; Cc1ccc(C(=O)CN2C3=C(CCCC3)SC2N)cc1]

siRNAs, shRNAs, miRNAs and antisense DNAs/RNAs

Inhibition (e.g. of the p53) may be achieved using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67:227-64). However, this response can be bypassed by using 21 nt siRNA duplexes (Elbashir et al. (2001) EMBO J. 20:6877-88; Hutvagner et al. (2001) Science 293:834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art.

### Adenoviral Proteins

Adenoviruses are natural co-helpers of AAV infection and provide a set of genes: E1a, E1b, E2a and E4 which optimize AAV infection.

The delivery of adenoviral proteins during gene editing may improve the efficiency of gene editing.

In one embodiment, the combination of the invention comprises an adenoviral protein or a nucleic acid sequence encoding an adenoviral protein. The combination may comprise more than one adenoviral protein. The adenoviral protein is not limited to a particular Adenovirus serotype. For example, in one embodiment, the adenoviral protein is from an Adenovirus of serotype 4, Adenovirus of serotype 5, Adenovirus of serotype 7 and/or Adenovirus of serotype 9.

In one embodiment, the adenoviral protein is from an Adenovirus of serotype 5.

In one embodiment, the adenoviral protein is selected from the group comprising E1a, E1b, E2a and E4.

In a preferred embodiment, the adenoviral protein is an open reading frame of the E4 gene.

In a one embodiment, the adenoviral protein is E4orf1 or a variant thereof.

An example of a nucleotide sequence encoding Ad5-E4orf1 is set forth in SEQ ID No. 4.

(SEQ ID NO. 4)
ATGGCCGCTGCTGTGGAAGCCCTGTACGTGGTGCTTGAAAGAGAGGGCG
CCATCCTGCCTAGACAAGAGGGCTTTTCTGGCGTGTACGTGTTCTTCAG
CCCCATCAACTTCGTGATCCCTCCAATGGGCGCCGTGATGCTGAGCCTG
AGACTGAGAGTGTGTATCCCTCCTGGCTACTTCGGCCGGTTTCTGGCCC
TGACCGATGTGAACCAGCCTGACGTGTTCACCGAGAGCTACATCATGAC
CCCTGACATGACCGAGGAACTGAGCGTGGTGCTGTTCAACCACGGCGAC
CAGTTCTTTTATGGCCACGCCGGAATGGCCGTCGTGCGGCTGATGCTGA
TCAGAGTGGTGTTTCCCGTCGTCCGGCAGGCCAGCAATGTTTGA.

An example of an amino acid sequence of Ad5-E4orf1 is set forth in SEQ ID No. 5. Suitably, the at least one adenoviral protein may comprise an amino acid sequence as set forth in SEQ ID No. 5 or a variant thereof.

(SEQ ID NO. 5)
MAAAVEALYVVLEREGAILPRQEGFSGVYVFFSPINFVIPPMGAVMLSL
RLRVCIPPGYFGRFLALTDVNQPDVFTESYIMTPDMTEELSVVLFNHGD
QFFYGHAGMAVVRLMLIRVVFPVVRQASNV

Other examples of an amino acid sequence of E4orf1 are set forth in SEQ ID No. 6 to SEQ ID No. 25. Suitably, the at least one adenoviral protein may comprise an amino acid sequence as set forth in SEQ ID No. 6 to SEQ ID No. 25 or a variant thereof.

| | | |
|---|---|---|
| SEQ ID No. 6 | Ad1-E4orf1 | MAAAVEALYVVLEREGAILPRQEGFSGVYVFFSPINFVIPPMGA |
| | | VMLPLRLRVCIPPGYFGRFLALTDVNQPDVFTESYIMTPDMTEE |
| | | LSVVLFNHGDQFFYGHAGMAVVRIMLIRVVFPVVRQASNV |
| SEQ ID No. 7 | Ad2-E4orf1 | MAAAVEALYVVLEREGAILPRQEGFSGVYVFFSPINFVIPPMGA |
| | | VMLSLRLRVCIPPGYFGRFLALTDVNQPDVFTESYIMTPDMIEE |
| | | LSVVLFNHGDQFFYGHAGMAVVRLMLIRVVFPVVRQASNV |
| SEQ ID No. 8 | Ad3-E4orf1 | MADEALYVYLEGPGATLPEQQQRNNYIFYSPVPFTLYPRGVALL |
| | | YLKLSIIIPRGYVGCFFSLTDANMSGLYASSRIIHAGHREELSV |
| | | LLFNHNDRFYEGRAGDPVACLVMERLIFPPVRQATMI |
| SEQ ID No. 9 | Ad4-E4orf1 | MDAQVLYVFLEGAGALLPVQKGSNYIFYAPANFVLHPHGVALLE |
| | | LRLSIVVPQGFIGRFFSLTDANVPGVYASSRIIHAGHREGLSVM |
| | | LFNHNVSFYNGRAGDPVACLVLERVIYPPVRQASMV |
| SEQ ID No. 10 | Ad6-E4orf1 | MAAAVEALYVVLEREGAILPRQEGFSGVYVFFSPINFVIPPMGA |
| | | VMLSLRLRVCIPPGYFGRFLALTDVSQPDVFTESYIMTPDMTEE |
| | | LSVVLFNHGDQFFYGHAGMAVVRLMLIRVVFPVVRQASNV |
| SEQ ID No. 11 | Ad7-E4orf1 | MADEALYVYLEGPGATLPEQQQRNNYIFYSPVPFTLYPRGVALL |
| | | YLRLSIIIPRGYVGCFFSLTDANMSGLYASSRIIHAGHREELSV |
| | | LLFNHDDRFYEGRAGDPVACLVMERLIYPPVRQATMI |
| SEQ ID No. 12 | Ad9-E4orf1 | MAESLYAFIDSPGGIAPVQEGTSNRYTFFCPESFHIPPHGVVLL |
| | | HLKVSVLVPTGYQGRFMALNDYHARDILTQSDVIFAGRRQELTV |
| | | LLFNHTDRFLYVRKGHPVGTLLLERVIFPSVKIATLV |
| SEQ ID No. 13 | Ad14-E4orf1 | MADEALYVYLEGPGATLPEQQQRNNYIFYSPVPFTLYPRGVALL |
| | | YLRLSIIIPRGYIGCFLSLTDANMFGLYASSRIIHAGHREELSV |
| | | LLFNHDDRFYEGRAGDPVACLVMERLIYPPVRQATLI |
| SEQ ID No. 14 | Ad16-E4orf1 | MADEALYVYFRGPGATLPEQQQQRNNYIFYSPVPFTLYPRGVAL |
| | | LYLRLSIIIPRGYVGCFFSLTDANMSGLYASSRIIHAGHREELS |
| | | VLLFNHDDRFYEGRAGDPVACLVMERLIYPPVRQATMI |
| SEQ ID No. 15 | Ad18-E4orf1 | MAALQALYVYFKGPGAMLPEQEGYSNAYVLESPANFVIPPHGVV |
| | | LLYLHIAVDIPPGYLGTLFSLSDMNARGVFVGAETLYPGSRMEL |
| | | SVLLFNHSDVFCDVRAKQPVARLLLSRVIFPPVRQASLL |
| SEQ ID No. 16 | Ad20-E4orf1 | MAESLYAFIDSPGGIAPVQEGTSNRYNFFCPQSFHIPPHGVVLL |
| | | HLKVSVLVPTGYQGRFMALNDYHARDILTQSDVIFAGRRQELTV |
| | | LLFNHTDRFLYVRKGHPVGTLLLERVIFPSVKIATLV |
| SEQ ID No. 17 | Ad21-E4orf1 | MAEVLYVILEGPGARLPVQEGNNYIFYAPVDFTLHPRGVALLHL |
| | | RLSIIVPRCYIGRFFSLTDTNTSGLYASSQIIFAAHQQPLSVML |
| | | FNHTDRFYEGRVGDPVACLVLERVIYPSVRQASMM |
| SEQ ID No. 18 | Ad23-E4orf1 | MAESLYAFIDSPGGIAPVQEGSSNRYNFFCPESFHIPPHGVVLL |
| | | HLRVSVLIPTGYQGRFMALNDYHARGILTQSDVIFAGRRHELTV |
| | | LLFNHTDRFLYVREGHPVGTLLLERVIFPSVRLATLV |
| SEQ ID No. 19 | Ad25-E4orf1 | MAESLYAFIDSPGGIAPVQEGTSNRYTFFCPESFHIPPHGVVLV |
| | | HLRVSVLIPNGYQGRFMALNDYHSRGILTQSDVIFAGRRQELTV |
| | | LLFNHTDRFLYVREGHPVGTLLLERVIFPSVRLATLV |
| SEQ ID No. 20 | Ad30-E4orf1 | MAESLYAFIDSPGGIAPVQEGASNRYTFFCPESFHIPPHGVILL |
| | | HLRVSVLVPTGYQGRFMALNDYHARGILTQSDVIFAGRRHDLSV |
| | | LLFNHTDRFLYVREGHPVGTLLLERVIFPSVRLATLV |
| SEQ ID No. 21 | Ad36-E4orf1 | MAESLYAFIDSPGGIAPVQEGASNRYIFFCPESFHIPPHGVILL |
| | | HLRVSVLVPTGYQGRFMALNDYHARGILTQSDVIFAGRRHDLSV |
| | | LLFNHTDRFLYVREGHPVGTLLLERVIFPSVRIATLV |
| SEQ ID No. 22 | Ad50-E4orf1 | MADEALYVYLDGPGATLPEQQQRNNYIFYSPVPFTLYPRGVALL |
| | | YLRLSIIIPRGYVGCFFSLTDANMSGLYASSRIIHAGHREELSV |
| | | LLFNHDDRFYEGRAGDPVACLVMERLIYPPVRQATMI |
| SEQ ID No. 23 | Ad55-E4orf1 | MAHEALYVYLEGPGATLPEQQQRNNYIFYSPVPFTLYPRGVALL |
| | | YLRLSIIIPRGYIGCFLSLTDANMFGLYASSRIIHAGHREELSV |
| | | LLFNHDDRFYEGRAGDPVACLVMERLIYPPVRQATLI |
| SEQ ID No. 24 | Ad62-E4orf1 | MAESLYAFIDSPGGIAPVQEGASNRYIFFCPESFHIPPHGVILL |
| | | HLRVSVMVPTGYQGRFMALNDYHARGILTQSDVIFAGRRHDLSV |
| | | LLFNHTDRFLYVREGHPVGTLLLERVIFPSVRIATLV |
| SEQ ID No. 25 | Ad71-E4orf1 | MAESLYAFIDSPGGIAPVQEGTSNRYDFFCPESFHIPPHGVVLL |
| | | HLRVSVLIPTGYQGRFMALNDYHARGILTQSDVIFAGRRHELTV |
| | | LLFNHTDRFLYVREGHPVGTLLLERVIFPSVRLATLV |

In a preferred embodiment, the adenoviral protein is E4orf6/7 or a variant thereof.

An example of a nucleotide sequence encoding Ad5-E4orf6/7 is set forth in SEQ ID No. 26.

(SEQ ID NO. 26)
ATGACCACCAGCGGCGTGCCCTTCGGCATGACACTCAGACCTACCAGAA
GCCGGCTGAGCAGAAGAACCCCTTACAGCAGAGACAGGCTGCCTCCATT
CGAGACAGAGACACGGGCCACCATCCTGGAAGATCACCCTCTGCTGCCC
GAGTGTAACACCCTGACCATGCACAACGCCTGGACAAGCCCATCTCCTC
CAGTGAAACAGCCCCAAGTGGGACAGCAGCCTGTTGCTCAGCAGCTGGA
CAGCGACATGAACCTGTCTGAACTGCCCGGCGAGTTCATCAACATCACC
GACGAGAGACTGGCCCGGCAAGAGACAGTGTGGAACATCACCCCTAAGA
ACATGAGCGTGACCCACGACATGATGCTGTTCAAGGCCAGCAGAGGCGA
GCGGACAGTGTACAGCGTTTGTTGGGAAGGCGGCGGACGGCTGAATACC
AGAGTGCTGTAA

An example of an amino acid sequence of Ad5-E4orf6/7 is set forth in SEQ ID No. 27. Suitably, the at least one adenoviral protein may comprise an amino acid sequence as set forth in SEQ ID No. 27 or a variant thereof.

(SEQ ID NO. 27)
MTTSGVPFGMTLRPTRSRLSRRTPYSRDRLPPFETETRATILEDHPLLP
ECNTLTMHNAWTSPSPPVKQPQVGQQPVAQQLDSDMNLSELPGEFINIT
DERLARQETVWNITPKNMSVTHDMMLFKASRGERTVYSVCWEGGGRLNT
RVL

In some embodiments, the adenoviral protein comprises or consists of the sequence SEQ ID NO: 27, or comprises or consists of an amino acid sequence having at least 70%, 75%, 80%, 85%, 90%, 95%, 96%, 97%, 98%, or 99% sequence identity to SEQ ID NO: 27.

Other examples of an amino acid sequence of E4orf6/7 are set forth in SEQ ID No. 29 to SEQ ID No. 59. Suitably, the at least one adenoviral protein may comprise an amino acid sequence as set forth in SEQ ID No. 29 to SEQ ID No. 59 or a variant thereof.

| | | |
|---|---|---|
| SEQ ID No. 29 | Ad1-E4orf6/7 | MTTSGVPFGMTLRPTRSRLSRRTPYSRDRLPPFETETRATILEDH |
| | | PLLPECNTLTMHNAWTSPSPPVEQPQVGQQPVAQQLDSNMNLREL |
| | | PGEFINITDERLARQETVWNITPKNMSVTHDMMLFKASRGERTVY |
| | | SVCWEGGGRLNTRVL* |
| SEQ ID No. 30 | Ad2-E4orf6/7 | MTTSGVPFGMTLRPTRSRLSRRTPYSRDRLPPFETETRATILEDH |
| | | PLLPECNTLTMHNAWTSPSPPVEQPQVGQQPVAQQLDSDMNLSEL |
| | | PGEFINITDERLARQETVWNITPKNMSVTHDMMLFKASRGERTVY |
| | | SVCWEGGGRLNTRVL* |
| SEQ ID No. 31 | Ad3-E4orf6/7 | MSGSNSIMTRLRARSTSCARHHPYTRAQLPRCEENETRASMTEDH |
| | | PLLPDCDTMTMHSMTVIQTPESHPQQLDCESALKDYPDAFLCITD |
| | | PRLARSETVWNVESKTMSISNGIQMFKAVRGERLVYSVKWEGGGK |
| | | ITTRIL* |
| SEQ ID No. 32 | Ad4-E4orf6/7 | MSGNSSIMTRSRTRLALSRHHPYQPPATLPRCEETESRASLVEDH |
| | | PVLPDCDTLSMHNITVIPITEDSPQLLNCEVQMQECPEGFISLTD |
| | | PRLSRSETVWNVEIKTMSITNSIQMFKAVRGERIVYSMRWEGGGK |
| | | ITTRIL* |
| SEQ ID No. 33 | Ad6-E4orf6/7 | MTTSGVPFGMTLRPTRSRLSRRTPYSRDRLPPFETETRATILEDH |
| | | PLLPECNTLTMHNAWTSPSPSVKRPQVGQQPVAQQLDSDMNLSEL |
| | | PGEFINITDERLARQETVWNITPKNMSVTHDMMLFKASRGERTVY |
| | | SVCWEGGGRLNTRVL* |
| SEQ ID No. 34 | Ad7-E4orf6/7 | MSGSNSIMTRLRARSTSCARHHPYTRAQLPRCEENETRASMTEDH |
| | | PLLPDCDTMTMHSMTVIQTPESHPQQLDCESALKDYPDAFLCITD |
| | | PRLARFETVWNVESKTMSICNGIQMFKAVRGERLVYSVKWEGGGK |
| | | ITTRIL* |
| SEQ ID No. 35 | Ad11-E4orf6/7 | MSGSNSIMTRLRARSTSCARHHPYTRAQLPRCEENETRASMTEDH |
| | | PLLPDCDTMTMHSMTVIQTPESQPQQLDCESALKDYPDAFLSITD |
| | | PRLARSETVWNVESKTMSISNGIQMFKAVRGERLVYSVKWEGGGK |
| | | ITTRIL* |
| SEQ ID No. 36 | Ad14-E4orf6/7 | MSGSNSIMTRLCARSTSCARHHPYTRAQLPRCEENETRASMTEDH |
| | | PLLPDCDTMTMHSMTVIQTPESHPQQLDCESALKDYPAGELSITD |
| | | PRLARYETVWNVESKTMSISNGIQMFKAVRGERLVYSVKWEGGGK |
| | | ITTRSL* |
| SEQ ID No. 37 | Ad18-E4orf6/7 | MQRNRRYPYRLAPYGKYPLPPCEKEMRASLFGPENSSVSECNSLT |
| | | LHNVINMDLVLDGESYLSDCVGEGFVSIIDHRFARKETIWTVTPK |
| | | NLSRNMHMQLFSAIKGERVVYKIKWEGGGSLTTRIV* |
| SEQ ID No. 38 | Ad19-E4orf6/7 | MQTEDQSSLLRHHPYRRARLPRSDEETRASLTEQHPLLPDCDHAD |
| | | YHNTVTLDCEARLDDFSEDGFISITDPRLARQETVWIIDPKLSSR |
| | | TNQNIPLFKATRAERTVYTVKWAGGGRLTTRAGVKINKDT* |
| SEQ ID No. 39 | Ad20-E4orf6/7 | MQTEIQSSSLRHHPYRRARLPRSDEETRASLTEQHPLLPDCDHAD |
| | | YHNTVTLDCEARLDDFSEDGFISITDPRLARQETVWIIDPKSSSR |
| | | TNQNISLFKATRAERTVYTVKWAGGGRLTTRAGVKINKDT* |
| SEQ ID No. 40 | Ad23-E4orf6/7 | MSTEEQSSSLRHHPYRRARLPRCEEETRASLTEQHPLLPDCDHAD |
| | | YHNTVTLDCEARLDDFSEDGFISITDPRLARQETVWIIDTKTSSR |
| | | TNNNIPLFKATRAERIVYTVKWAGGGRLTTRAGVKINKDT* |
| SEQ ID No. 41 | Ad25-E4orf6/7 | MSTEEQSSSLRHHPYRRARLPRCEEETRASLTEQHPLLPDCDHAD |
| | | YHNTVTLDCEARLDDFSEDGFISITDPRLARQETVWIIDPKSSSR |
| | | TNQNISLFKATRAERTVYTVKWAGGGRLTTRAGVKINKDT* |
| SEQ ID No. 42 | Ad27-E4orf6/7 | MQTEDQSSLLRHHPYRRARLPRSDEETRASLTEQHPLLPDCDHAD |
| | | YHNTVTLDCEARLDDFSEDGFISITDPRLARQETVWIIDPKESSR |
| | | TNQNIPLFKATRAERIVYTVKWAGGGRLTTRAGVKINKDT* |
| SEQ ID No. 43 | Ad29-E4orf6/7 | MQTEDQSSLLRHHPYRRARLPRSDEETRASLTEQHPLLPDCDHAE |
| | | YHNTVTLDCEARLDDFSEDGFISITDPRLARQETVWLIDTKSSSR |
| | | ANQNIPLFKATRAERIVYTVKWAGGGRLTTRAGVKINKDT* |
| SEQ ID No. 44 | Ad30-E4orf6/7 | MSTEEQSSSLRHHPYRRARLPRCEEETRASLTEQHPLLPDCDHAD |
| | | YHNTVTLDCEARLDDFSEDGFISITDPRLARQETVWIIDSKSSSR |
| | | TNQNIPLFKATRAERIVYTVKWAGGGRLTTRAGVKINKDP* |
| SEQ ID No. 45 | Ad32-E4orf6/7 | MSTEEQSSSLRHHPYRRARLPRCDEETRASLTEQHPLLPDCDHAD |
| | | YHNTVTLDCEARLDDFSEDGFISITDPRLARQETVWIIDPKSSSR |
| | | TNQNIFLFKATRAERIVYTVKWAGGGRLTTRAGVKINKDT* |
| SEQ ID No. 46 | Ad34-E4orf6/7 | MSGSNSIMTRLRARSTSCARHHPYTRAQLPRCEENETRASMTEDH |
| | | PLLPDCDTMTMHSMTVIQTPESHPQQLDCESALKDYPDAFLSITD |
| | | PRLARSETVWNVESKTMSISNGIQMFKAVRGERLVYSVKWEGGGK |
| | | ITTRIL* |
| SEQ ID No. 47 | Ad36-E4orf6/7 | MSTEEQSTSLRHHPYRRARLPRSEEETRASLTEQHPLLPDCDHAE |
| | | YHNTVTLDCEARLEDFSEDGFISITDPRLARQETVWIIDTKSSSR |
| | | TNQNIPLFKATRAERIVYTVKWAGGGRLTTRAGVKINKDT* |
| SEQ ID No. 48 | Ad38-E4orf6/7 | MSTEEQSSLLRHHPYRRARLPRCEEETRASLTEQHPLLPDCDHAD |
| | | YHNTVTLDCEARLDDFSEDGFISITDPRLARQETVWIIDTKSSSR |
| | | SNNNIPLFKATRAERIVYTVKWAGGGRLTTRAGVKINKDS* |
| SEQ ID No. 49 | Ad39-E4orf6/7 | MSTEEQSSLLRHHPYRRARLPRCEEETRASLTEQHPLLPDCDHAD |
| | | YHNTVTLDCEARLEDFSEDGFISITDPRLARQETVWIIDTKSSSR |
| | | TNQNIPLFKATRAERIVYTVKWAGGGRLTTRAGVKINKDT* |
| SEQ ID No. 50 | Ad43-E4orf6/7 | MSTEEQSSLLRHHPYRRARLPRCEQETRASLTEQHPLLPDCDHAE |
| | | YHNTVTLDCEPRLEDFSEDGFISITDPRLARQETVWLIDTKTSSR |
| | | TNQNIPLFKATRAERTVYTVKWAGGGRLTTRAGVKINKDT* |
| SEQ ID No. 51 | Ad44-E4orf6/7 | MQTEDQSSLLRHHPYRRARLPRCEEETRASLTEQHPLLPDCDHAD |
| | | YHNTVTLDCEARLEDFSEDGFISITDPRLARQETVWLIDTKSSSR |
| | | TNQNIPLFKATRAERTVYTVKWAGGGRLTTRAGVKINKDT* |
| SEQ ID No. 52 | Ad45-E4orf6/7 | MSTEEQSSLLRHHPYRRARLPRCEEETRASLTEQHPLLPDCDHAD |
| | | YHNTVTLDCEARLDDFSEDGFISITDPRLARQETVWIIDPKSSSR |
| | | TNQNISLFKATRAERIVYTVKWAGGGRLTTRAGVKINKDT* |
| SEQ ID No. 53 | Ad47-E4orf6/7 | MSTEEQSSLLRHHPYRRARLPRSDEETRASLTEQHPLLPDCDHAD |
| | | YHNTVTLDCEARLEDFSEDGFISITDPRLARQETVWIIDPKSSSR |
| | | TNQNIPLFKATRAERIVYTVKWAGGGRLTTRAGVKINKDT* |
| SEQ ID No. 54 | Ad50-E4orf6/7 | MSGNNSIMTRLRARSTSCARHHPYTRAQLPRCEENETRASMTEDH |
| | | PLLPDCDTMTMHSMTVIQTPESHPQQLDCESALKDYPDAFLCITD |
| | | PRLARSETVWNVETKTMSISNGIQMFKAVRGERLVYSVKWEGGGK |
| | | ITTRIL* |
| SEQ ID No. 55 | Ad53-E4orf6/7 | MQTEIQSSSLRHHPYRRARLPRSDEETRASLTEQHPLLPDCDHAD |
| | | YHNTVTLDCEARLDDFSEDGFISITDPRLARQETVWIINPKSSSR |
| | | TNENFPLFKATRAERIVYTVKWAGGGRMCTRAGVKINKDT* |
| SEQ ID No. 56 | Ad54-E4orf6/7 | MSTEEQSTLLRHHPYRRARLSRYDKETRASLTEQHPLLPDCDHAD |
| | | YHNTVTLDCEARLDDLSKDGFISITDPRLARQETVWIIDPKSSSR |
| | | TNENFPLFKATRTERIVYTVKWAGGGRMCTRAGVKINKDT* |
| SEQ ID No. 57 | Ad58-E4orf6/7 | MQTEIQSSLLRHHPYRRARLPRSDEETRASLTEQHPLLPDCDHAD |
| | | YHNTVTLDCEARLDDFSEDGFISITDPRLARQETVWLIDTKSSSR |
| | | ANQNIPLFKATRAERIVYTVKWAGGGRLTTRAGVKINKDT* |
| SEQ ID No. 58 | Ad64-E4orf6/7 | MQTEIQSSSLRHHPYRRARLPRSDEETRASLTEQHPLLPDCDHAD |
| | | YHNTVTLDCEARLDDFSEDGFISITDPRLARQETVWIIDPKSNSR |
| | | TNENISLFKATRAERTVYTVKWAGGGRLSTRAGVKINKDT* |
| SEQ ID No. 59 | Ad69-E4orf6/7 | MQTEIQSSSLRHHPYRRARLPRSDEETRASLTEQHPLLPDCDHAD |
| | | YHNTVTLDCEARLDDFSEDGFISITDPRLARQETVWIIDPKSSSR |
| | | TNQNISLFKATRAERIVYTVKWAGGGRLTTRAGVKINKDT* |

Variant sequences of SEQ ID NOs recited herein may, for example, have at least 70%, 75%, 80%, 85%, 90%, 95%, 98% or 99% sequence identity to the reference sequence SEQ ID NOs. Preferably, the variant sequence retains one or more functions of the reference sequence (i.e. is a functional variant).

Variant sequences may comprise one or more conservative substitutions. Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) variants i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below.

An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue. Suitably, a conservative substitution may be made between amino acids in the same line in the Table above.

An example of a nucleotide sequence encoding E4orf6 is set forth in SEQ ID No. 60.

(SEQ ID No. 60)
ATGACAACCAGCGGCGTGCCCTTCGGCATGACACTGAGGCCTACAAGAA
GCCGGCTGAGCAGAAGAACCCCTTACAGCAGAGACAGACTGCCTCCATT
CGAGACAGAGACACGGGCCACCATCCTGGAAGATCACCCTCTGCTGCCC
GAGTGCAACACCCTGACAATGCACAACGTGTCCTACGTGCGGGGCCTGC
CTTGTAGCGTTGGCTTCACACTGATCCAAGAGTGGGTCGTGCCCTGGGA
CATGGTGCTGACCAGAGAGGAACTGGTCATCCTGCGGAAGTGTATGCAC
GTGTGCCTGTGCTGCGCCAACATCGACATCATGACCAGCATGATGATCC
ACGGCTACGAGAGCTGGGCCCTGCACTGTCACTGTTCTAGCCCTGGCAG
CCTGCAGTGTATTGCTGGTGGACAGGTTCTGGCCAGCTGGTTCAGAATG
GTGGTGGACGGCGCCATGTTCAACCAGAGATTCATCTGGTACAGAGAGG
TGGTCAACTACAACATGCCCAAAGAAGTGATGTTCATGAGCAGCGTTTT
CATGCGGGGCAGACACCTGATCTACCTGCGGCTTTGGTACGATGGCCAC
GTGGGATCTGTGGTGCCTGCCATGAGCTTTGGCTACAGCGCCCTGCATT
GCGGCATCCTGAACAACATCGTGGTGCTGTGCTGCAGCTACTGCGCCGA
TCTGAGCGAGATCAGAGTGCGGTGTTGTGCCAGACGGACCAGACGGCTG
ATGCTGAGAGCCGTGCGGATCATTGCCGAAGAGACAACCGCCATGCTGT
ACTCTTGCCGGACCGAGAGAAGAAGGCAGCAGTTCATCAGAGCCCTGCT
CCAGCACCACCGGCCTATCCTGATGCACGACTACGACAGCACCCCTATG
TAG

An example of an amino acid sequence of E4orf6 is set forth in SEQ ID No. 61.

(SEQ ID No. 61)
MTTSGVPFGMTLRPTRSRLSRRTPYSRDRLPPFETETRATILEDHPLLP
ECNTLTMHNVSYVRGLPCSVGFTLIQEWVVPWDMVLTREELVILRKCMH
VCLCCANIDIMTSMMIHGYESWALHCHCSSPGSLQCIAGGQVLASWFRM
VVDGAMFNQRFIWYREVVNYNMPKEVMFMSSVFMRGRHLIYLRLWYDGH
VGSVVPAMSFGYSALHCGILNNIVVLCCSYCADLSEIRVRCCARRTRRL
MLRAVRIIAEETTAMLYSCRTERRRQQFIRALLQHHRPILMHDYDSTPM

An example of a nucleotide sequence encoding E1B55K is set forth in SEQ ID No. 62.

(SEQ ID No. 62)
ATGGAAAGACGGAACCCCAGCGAGAGAGGCGTGCCAGCTGGATTTTCTGGACACGCCAGCGTGGAAAGCGGCTGC
GAGACACAAGAAAGCCCTGCCACCGTGGTGTTCAGACCACCTGGCGATAATACCGATGGCGGAGCTGCTGCTGCA
GCTGGTGGATCTCAGGCAGCAGCTGCAGGCGCTGAACCTATGGAACCTGAGAGCAGACCTGGACCTAGCGGCATG
AATGTGGTGCAGGTCGCCGAGCTGTATCCCGAGCTGAGAAGAATCCTGACCATCACCGAGGATGGCCAGGGACTG
AAGGGCGTGAAGAGAGAAAGAGGCGCCTGCGAGGCCACAGAGGAAGCCAGAAATCTGGCCTTCAGCCTGATGACC
AGACACAGACCCGAGTGCATCACCTTCCAGCAGATCAAGGACAACTGCGCCAACGAGCTGGACCTGCTGGCCCAG
AAGTACAGCATCGAGCAGCTGACCACCTACTGGCTGCAACCCGGCGACGATTTCGAAGAGGCCATCAGAGTGTAC
GCCAAGGTGGCCCTCAGACCTGACTGCAAGTACAAGATCAGCAAGCTGGTCAACATCCGGAACTGCTGCTACATC
AGCGGCAATGGCGCCGAGGTGGAAATCGACACAGAGGACAGAGTGGCCTTCCGGTGCAGCATGATCAACATGTGG
CCTGGCGTGCTCGGCATGGATGGCGTGGTCATTATGAACGTGCGGTTCACAGGCCCCAACTTCAGCGGCACAGTG
TTTCTGGCCAACACCAACCTGATCCTGCACGGCGTGTCCTTCTACGGCTTCAACAATACCTGCGTGGAAGCCTGG
ACCGACGTTCGCGTTAGAGGCTGCGCCTTCTACTGCTGTTGGAAGGGCGTCGTGTGCAGACCCAAGAGCAGAGCC
AGCATCAAGAAGTGCCTGTTCGAGAGATGCACCCTGGGCATCCTGAGCGAGGGCAACAGCAGAGTCAGACACAAC
GTGGCCAGCGACTGCGGCTGCTTCATGCTGGTTAAGAGCGTGGCCGTGATCAAGCACAACATGGTCTGCGGCAAC
TGCGAGGATAGAGCCAGCCAGATGCTGACCTGCAGCGACGGCAATTGTCATCTGCTGAAAACCATCCACGTGGCC
TCTCACAGCAGAAAGGCCTGGCCTGTGTTCGAGCACAATATCCTGACACGGTGCTCCCTGCACCTGGGCAATAGA
CGGGGAGTGTTCCTGCCTTACCAGTGCAACCTGAGCCACACCAAGATCCTGCTGGAACCCGAGTCCATGAGCAAA
GTGAACCTGAATGGCGTGTTCGACATGACCATGAAGATCTGGAAAGTGCTGCGCTACGACGAGACACGGACCAGA
TGTAGACCTTGCGAGTGTGGCGGCAAGCACATCAGAAACCAGCCTGTGATGCTGGACGTGACCGAGGAACTGAGG
CCTGATCATCTGGTGCTGGCCTGTACCAGAGCCGAGTTTGGCAGCTCCGACGAGGATACCGAT

An example of an amino acid sequence of E1B55K is set forth in SEQ ID No. 63.

(SEQ ID NO. 63)
MERRNPSERGVPAGFSGHASVESGCETQESPATVVFRPPGDNTDGGAAAAAGGSQAAAAGAEPMEPESRPGPSGM
NVVQVAELYPELRRILTITEDGQGLKGVKRERGACEATEEARNLAFSLMTRHRPECITFQQIKDNCANELDLLAQ
KYSIEQLTTYWLQPGDDFEEAIRVYAKVALRPDCKYKISKLVNIRNCCYISGNGAEVEIDTEDRVAFRCSMINMW
PGVLGMDGVVIMNVRFTGPNFSGTVFLANTNLILHGVSFYGFNNTCVEAWTDVRVRGCAFYCCWKGVVCRPKSRA
SIKKCLFERCTLGILSEGNSRVRHNVASDCGCFMLVKSVAVIKHNMVCGNCEDRASQMLTCSDGNCHLLKTIHVA
SHSRKAWPVFEHNILTRCSLHLGNRRGVFLPYQCNLSHTKILLEPESMSKVNLNGVFDMTMKIWKVLRYDETRTR
CRPCECGGKHIRNQPVMLDVTEELRPDHLVLACTRAEFGSSDEDTD

In one embodiment, the at least one adenoviral protein is not E4orf6. In one embodiment, the at least one adenoviral protein is not E1B55K. In one embodiment, the at least one adenoviral proteins does not comprise E4orf6 or E1B55K.

Other transduction enhancers

The combination of CsH or a derivative thereof, and a p53 inhibitor and/or an adenoviral protein may be used in combination with any other additional transduction enhancer.

As used herein, a “transduction enhancer” may refer to any agent capable of increasing the efficiency of transduction. Exemplary transduction enhancers include enhancers of prostaglandin EP receptor signalling (e.g. PGE2, dmPGE2, derivatives, analogues and precursors of PGE2); CAMP activators (e.g. cAMP/PI3K/AKT agonists); ABC transporter inhibitors (e.g. verapamil, quinidine, diltiazem, ritonavir); mTOR inhibitors (e.g. rapamycin or a derivative thereof); beta-deliverin; inhibitors of cofilin phosphorylation (e.g. Staurosporin); CsA and derivatives thereof; LentiBOOST and other poloxamers (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88); poloxamine compounds (e.g. T304, T701, T901, T904, T908, T1107, T1301, T1304, T1307, 9R4, 15R1); retronectin; vectofusin-1 and derivatives thereof; polybrene; protamine sulphater; DEAE-Dextran; human semen-derived enhancer of infection; HIVgp120-derived peptides; b1 receptor blockers and selective serotonin reuptake inhibitors; Vpx; nanofibrils (e.g. EF-C peptides); epigenetic drugs; proteosomal inhibitors; kinase and kinase receptor inhibtors; central DNA flap/PPT; and cationic lipids or recombinant fibronectin.

In one embodiment, the at least one additional transduction enhancer is selected from: an enhancer of prostaglandin EP receptor signalling (e.g. PGE2, dmPGE2, derivatives, analogues and precursors of PGE2); a CAMP activator (e.g. cAMP/PI3K/AKT agonists); an ABC transporter inhibitor (e.g. verapamil, quinidine, diltiazem, ritonavir); a mTOR inhibitor (e.g. rapamycin or a derivative thereof); beta-deliverin; an inhibitor of cofilin phosphorylation (e.g. Staurosporin); CsA or a derivative thereof; LentiBOOST or other poloxamer (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88); a poloxamine compound (e.g. T304, T701, T901, T904, T908, T1107, T1301, T1304, T1307, 9R4, 15R1); retronectin; vectofusin-1 or a derivative thereof; polybrene; protamine sulphate; DEAE-Dextran; human semen-derived enhancer of viral infection (SEVI); a HIV gp120-derived peptide; a b1 receptor blocker or a selective serotonin reuptake inhibitor; Vpx; a nanofibril (e.g. an EF-C peptide); an epigenetic drug; a proteosome inhibitor; a kinase or kinase receptor inhibitor; a central DNA flap or PPT; a cationic lipid or a recombinant fibronectin.

In one embodiment, the at least one additional transduction enhancer comprises an enhancer of prostaglandin EP receptor signalling (e.g. PGE2, dmPGE2, derivatives, analogues and precursors of PGE2). Prostaglandin E2 (PGE2), which is also known as dinoprostone, is a naturally occurring prostaglandin having the structure:

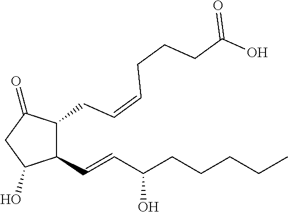 [SMILES: CCCCC[C@H](O)/C=C/[C@H]1[C@H](O)CC(=O)[C@@H]1C/C=C\CCCC(=O)O]

Prostaglandin E2 or a prostaglandin E2 derivative may be used for increasing transduction efficiency and/or gene editing efficiency of an isolated population of cells. In one embodiment, the prostaglandin E2 derivative is 16, 16-dimethyl prostaglandin E2. By derivative of prostaglandin E2, it is to be understood that prostaglandin E2 is modified by any of a number of techniques known in the art, preferably to improve properties such as stability and activity, while still retaining its function of increasing transduction efficiency and/or gene editing efficiency of an isolated population of cells. WO2007112084, for example, describes agents that stimulate the PGE2 pathway.

In one embodiment, the at least one additional transduction enhancer comprises dmPGE2. In one embodiment, the dmPGE2 is present at a concentration of around 1 to 50 μM, preferably 10 μM.

In one embodiment, the at least one additional transduction enhancer comprises LentiBOOST. In one embodiment, the LentiBOOST is present at a concentration of around 0.5 to 5 mg/ml, preferably 1 mg/ml.

In one embodiment, the at least one additional transduction enhancer comprises dmPGE2 (e.g. at a concentration of 10 μM) and LentiBOOST (e.g. at a concentration of 1 mg/ml).

In one embodiment, the at least one additional transduction enhancer comprises a cAMP activator (e.g. cAMP/PI3K/AKT agonists). WO2013049615, for example, describes compounds that stimulate the prostaglandin EP receptor signaling pathway by increasing signaling through the cAMP/P13K/AKT second messenger pathway.

In one embodiment, the at least one additional transduction enhancer comprises an ABC transporter inhibitor (e.g. verapamil, quinidine, diltiazem, ritonavir). WO2004098531, for example, describes increased transduction using ABC transporter inhibitors.

In one embodiment, the at least one additional transduction enhancer comprises an mTOR inhibitor (e.g. rapamycin or a derivative thereof). Rapamycin (CAS No. 53123-88-9, also known as Sirolimus) is a macrolide produced by Streptomyces hygroscopicus. Rapamycin has the following structure:

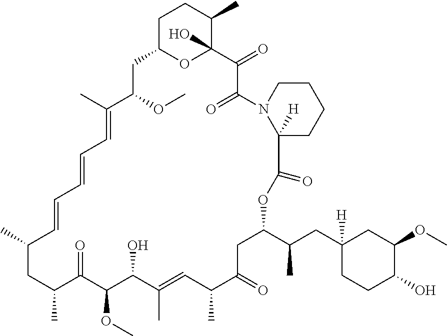

Rapamycin is an approved immunosuppressive agent for use in prevention of allograft rejection. By derivative of rapamycin, it is to be understood that rapamycin is modified by any of a number of techniques known in the art, preferably to improve properties such as stability and activity, while still retaining its function of increasing transduction efficiency and/or gene editing efficiency of an isolated population of cells.

In one embodiment, the at least one additional transduction enhancer comprises beta-deliverin.

In one embodiment, the at least one additional transduction enhancer comprises an inhibitor of cofilin phosphorylation (e.g. Staurosporin). Staurosporine is a natural product originally isolated from Streptomyces staurosporeus. It displays activity as an inhibitor of protein kinases through the prevention of ATP binding to the kinase.

In one embodiment, the at least one additional transduction enhancer comprises CsA or a derivative thereof. Petrillo C, et al (2015) Mol Ther 23:352-362 describes that cyclosporin A (CsA) and rapamycin relieve distinct lentiviral restriction blocks in hematopoietic stem and progenitor cells.

In one embodiment, the at least one additional transduction enhancer comprises LentiBOOST or another poloxamer (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88). WO2013127964, for example, describes retroviral transduction using poloxamers.

In one embodiment, the at least one additional transduction enhancer comprises a poloxamine compound (e.g. T304, T701, T901, T904, T908, T1107, T1301, T1304, T1307, 9R4, 15R1). WO2003066104, for example, describes using poloxamines to improve in vivo gene transfer.

In one embodiment, the at least one additional transduction enhancer comprises retronectin.

In one embodiment, the at least one additional transduction enhancer comprises vectofusin-1 or a derivative thereof.

In one embodiment, the at least one additional transduction enhancer comprises polybrene.

In one embodiment, the at least one additional transduction enhancer comprises protamine sulphate.

In one embodiment, the at least one additional transduction enhancer comprises DEAE-Dextran.

In one embodiment, the at least one additional transduction enhancer comprises human semen-derived enhancer of viral infection (SEVI).

In one embodiment, the at least one additional transduction enhancer comprises a HIV gp120-derived peptide.

In one embodiment, the at least one additional transduction enhancer comprises a b1 receptor blocker or a selective serotonin reuptake inhibitor.

In one embodiment, the at least one additional transduction enhancer comprises Vpx.

In one embodiment, the at least one additional transduction enhancer comprises a nanofibril (e.g. an EF-C peptide).

In one embodiment, the at least one additional transduction enhancer comprises an epigenetic drug.

In one embodiment, the at least one additional transduction enhancer comprises a proteosome inhibitor.

In one embodiment, the at least one additional transduction enhancer comprises a kinase or kinase receptor inhibitor.

In one embodiment the at least one additional transduction enhancer comprises a central DNA flap or PPT.

In one embodiment, the at least one additional transduction enhancer comprises a cationic lipid or a recombinant fibronectin.

In one embodiment, the combination of the invention further comprises one or more additional agents. The one or more additional agents may include, for example, one or more cell culture supplement, including antibiotics (e.g. penicillin, streptomycin), amino acids (e.g. glutamine), carbohydrates (e.g. glucose, galactose, maltose, fructose, pyruvate), vitamins (e.g. vitamin B12, vitamin A, vitamin E, riboflavin, thiamine, biotin), inorganic salts (e.g. sodium salts, potassium salts, calcium salts), buffers (e.g. HEPES), proteins (e.g. albumin, transferrin, fibronectin, fetuin, growth factors), lipids and fatty acids (e.g. cholesterol, steroids), and trace elements (e.g. zince, copper, selenium).

Population of cells

In one aspect, the present invention provides a population of cells prepared according to the method of the invention.

In another aspect, the present invention provides a kit comprising the population of cells of the invention.

The population of cells may be an isolated population of cells. In one embodiment, the population of cells comprises, substantially consists of, or consists of: haematopoietic stem and/or progenitor cells (HSPCs), and/or peripheral blood mononuclear cells (PBMCs), and/or T cells.

In preferred embodiments the population of cells are quiescent. Quiescence is a reversible state of a cell in which it does not divide but retains the ability to re-enter cell proliferation. Some adult stem cells are maintained in a quiescent state and can be rapidly activated when stimulated.

### Haematopoietic Stem and Progenitor Cells

In one embodiment, the population of cells comprises, substantially consists of, or consists of haematopoietic stem and/or progenitor cells (HSPCs).

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

The haematopoietic stem and progenitor cells of the invention comprise the CD34 cell surface marker (denoted as CD34⁺). In one embodiment, the haematopoietic stem and/or progenitor cells comprise, substantially consist of, or consist of CD34⁺ cells or CD34-cells. In one embodiment, the haematopoietic stem and/or progenitor cells comprise, substantially consist of, or consist of primitive subtypes. In one embodiment, the haematopoietic stem and/or progenitor cells comprise, substantially consist of, or consist of CD34⁺ cells. In one embodiment, the haematopoietic stem and/or progenitor cells comprise, substantially consist of, or consist of CD34⁺ CD133-CD90-, CD34⁺ CD133⁺ CD90, and/or CD34⁺ CD133⁺ CD90⁺ cells. In one embodiment, the haematopoietic stem and/or progenitor cells comprise, substantially consist of, or consist of CD34⁺ CD133⁺ CD90⁺ cells.

### Haematopoietic Stem and Progenitor Cell (HSPC) Source

A population of haematopoietic stem and/or progenitor cells (HSPCs) may be obtained from a tissue sample.

For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, haematopoietic stem and progenitor cells may also be derived from induced pluripotent stem cells.

### Hsc Characteristics

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

### Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker” it is to be understood that human HSCs lack the expression of these markers.

### Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker” it is to be understood that human HSCs express these markers.

In one embodiment, the haematopoietic stem and progenitor cells are CD34⁺ CD38-cells.

In one embodiment, the HSPCs are pre-stimulated HSPCs. Pre-stimulated HSPCs may be HSPCs which are stimulated prior to transduction. In preferred embodiments, the HSPCs are stimulated before and/or during transduction. In one embodiment, the HSPCs are stimulated before transduction. In one embodiment, the HSPCs are stimulated during transduction. Cytokines for stimulating quiescent HSPCs are known to those of skill in the art and include, for example early-acting cytokines such as IL-3, IL-6, stem cell factor (SCF), and Flt-3L. In one embodiment, the HSPCs are contacted with cytokines (e.g. early-acting cytokines) before and/or during transduction. In one embodiment, the HSPCs are contacted with recombinant human stem cell factor (rhSCF), recombinant human thrombopoietin (rhTPO), recombinant human Flt3 ligand (rhFlt3), or recombinant human IL6 (rhIL6) before and/or during transduction.

### Peripheral Blood Mononuclear Cells and T Cells

In one embodiment, the population of cells comprises, substantially consists of, or consists of peripheral blood mononuclear cells (PBMCs). Peripheral blood mononuclear cells (PBMCs) are blood cells with round nuclei, such as monocytes, lymphocytes, and macrophages.

The PBMCs of the invention may, for example, not display the CD14 cell surface marker (denoted as CD14-). Cluster of differentiation 14 (CD14) has been described as a monocyte/macrophage differentiation antigen on the surface of myeloid lineage and has been commonly used in normal tissue or blood as a marker for myeloid cells.

In one embodiment, the population of cells comprises, substantially consists of, or consists of T cells. T cells (or T lymphocytes) are a type of lymphocyte that play a central role in cell-mediated immunity. They can be distinguished from other lymphocytes, such as B cells and natural killer cells (NK cells), by the presence of a T-cell receptor (TCR) on the cell surface.

In one embodiment, the T cells are resting T cells. Resting CD4⁺ T cells are quiescent. In one embodiment, the T cells are unstimulated T cells. Once stimulated, these resting T cells proliferate and generate a large clone of antigen-specific cells. In one embodiment, the T cells are CD4⁺ T cells. In one embodiment, the T cells are CD3⁺ T cells. In one embodiment, the T cells are CD8⁺ T cells.

In one embodiment, the T cells are resting CD3⁺ T cells. In one embodiment, the T cells are Stem memory T cells; Central Memory T cells; Effector Memory T cells; and/or terminally differentiated effector memory T cells.

Differentiated cells

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes. Here, a differentiated cell includes differentiated cells of the haematopoietic lineage such as monocytes, macrophages, neutrophils, basophils, eosinophils, erythrocytes, megakaryocytes/platelets, dendritic cells, T cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

### Isolation and Enrichment of Populations of Cells

The term “isolated population” of cells as used herein may refer to the population of cells having been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody.

The term “antibody” refers to complete antibodies or antibody fragments capable of binding to a selected target, and including Fv, ScFv, F (ab′) and F(ab′) 2, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for haematopoietic stem cells.

Suitably, the agent does not reduce the fraction CD34⁺ CD133⁺ CD90⁺ cells in population of gene edited cells compared with a population of untreated gene edited cells.

### Gene Editing

The term “gene editing” refers to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations.

Such nucleases may be delivered to a target cell using viral vectors. The present invention provides methods of increasing the efficiency of the gene editing process.

Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31:397-405; Sander, J. D. et al. (2014) Nat. Biotechnol. 32:347-55).

Meganucleases (Silve, G. et al. (2011) Cur. Gene Ther. 11:11-27) may also be employed as suitable nucleases for gene editing.

The CRISPR/Cas system is an RNA-guided DNA binding system (van der Oost et al. (2014) Nat. Rev. Microbiol. 12:479-92), wherein the guide RNA (gRNA) may be selected to enable a Cas9 domain to be targeted to a specific sequence. Methods for the design of gRNAs are known in the art. Furthermore, fully orthogonal Cas9 proteins, as well as Cas9/gRNA ribonucleoprotein (RNP) complexes and modifications of the gRNA structure/composition to bind different proteins, have been recently developed to simultaneously and directionally target different effector domains to desired genomic sites of the cells (Esvelt et al. (2013) Nat. Methods 10:1116-21; Zetsche, B. et al. (2015) Cell pii: S0092-8674 (15) 01200-3; Dahlman, J. E. et al. (2015) Nat. Biotechnol. 2015 Oct. 5. doi: 10.1038/nbt.3390. [Epub ahead of print]; Zalatan, J. G. et al. (2015) Cell 160:339-50; Paix, A. et al. (2015) Genetics 201:47-54), and are suitable for use in the invention.

### Viral Vectors

A vector is a tool that allows or facilitates the transfer of an entity from one environment to another. The vectors used to transduce the cells in the present invention are viral vectors.

In one embodiment, the viral vectors are retroviral vectors. In a preferred embodiment, the viral vectors are lentiviral vectors.

In one embodiment, the lentiviral vectors are derived from HIV-1, HIV-2, SIV, FIV, BIV, EIAV, CAEV or visna lentivirus. In one embodiment, the viral vector is a gamma-retroviral vector.

The vector of the present invention may be in the form of a viral vector particle. In one embodiment, the viral vector is pseudotyped to enter cells via an endocytosis-dependent mechanism and/or the viral vector is a VSV-g pseudotyped vector. In one embodiment, the viral vector is pseudotyped to enter cells via an endocytosis-dependent mechanism. In one embodiment, the viral vector is a VSV-g pseudotyped vector. In one embodiment, the viral vector is a measles virus glycoprotein pseudotyped viral vector. In one embodiment, the viral vector is pseudotyped with measles virus glycoproteins hemagglutinin (H) and fusion protein (F). In one embodiment, the viral vector is not an adeno-associated virus (AAV) vector.

By “vector derived from” a certain type of virus, it is to be understood that the vector comprises at least one component part derivable from that type of virus.

### Retroviral and Lentiviral Vectors

A retroviral vector may be derived from or may be derivable from any suitable retrovirus. A large number of different retroviruses have been identified. Examples include murine leukaemia virus (MLV), human T cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from other retroviruses in that lentiviruses have the capability to infect both dividing and non-dividing cells (Lewis, P et al. (1992) EMBO J. 11:3053-8; Lewis, P. F. et al. (1994) J. Virol. 68:510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

The plasmid vector used to produce the viral genome within a host cell/packaging cell may also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272:263-7; Naldini, L. et al. (1996) Proc. Natl. Acad. Sci. USA 93:11382-8; Leavitt, A. D. et al. (1996) J. Virol. 70:721-8) or by modifying or deleting essential att sequences from the vector LTR (Nightingale, S. J. et al. (2006) Mol. Ther. 13:1121-32), or by a combination of the above. In one embodiment, the vector is integrase-defective.

### Hiv-Derived Vectors

In one embodiment, the viral vector is an HIV-derived vector. HIV-derived vectors for use in the present invention are not particularly limited in terms of HIV strain. Numerous examples of sequences of HIV strains may be found at the HIV Sequence Database (http://www.hiv.lanl.gov/content/index).

For example, a HIV-1-derived vector may be derived from any of the HIV-1 strains NL4-3, IIIB_LAI or HXB2_LAI (X4-tropic), or BAL (R5-tropic), or a chimaera thereof. Preferably, HIV-1-derived vectors are derived from the pMDLg/pRRE Gag-Pol-expressing packaging construct (U.S. Pat. Nos. 7,629,153; 8,652,837; Naldini, L. et al. (1996) Science 272:263-7; Follenzi, A. et al. (2002) Methods Enzymol. 346:454-65).

A HIV-2-derived vector may be derived, for example, from the HIV-2 strain ROD.

### Nucleotide of Interest

The vector used in the present invention preferably comprises a nucleotide of interest (NOI). Preferably, the nucleotide of interest gives rise to a therapeutic effect.

Suitable NOIs include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, guide RNA (gRNA, e.g. used in connection with a CRISPR/Cas system), ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The NOIs may also encode pro-drug activating enzymes.

An example of a NOI is the beta-globin chain which may be used for gene therapy of thalassemia/sickle cell disease.

NOIs also include those useful for the treatment of other diseases requiring non-urgent/elective gene correction in the myeloid lineage such as: chronic granulomatous disease (CGD, e.g. the gp91phox transgene), leukocyte adhesion defects, other phagocyte disorders in patients without ongoing severe infections and inherited bone marrow failure syndromes (e.g. Fanconi anaemia), as well as primary immunodeficiencies (SCIDs).

NOIs also include those useful in the treatment of lysosomal storage disorders and immunodeficiencies.

The applicability of the invention to T cells also facilitates its application in cell therapies that are based on infusion of modified T cells into patients, including anti-cancer strategies (such as using engineered CAR-T cells) and approaches based on infusion of universal donor T cells. NOIs may therefore also include, for example, chimeric antigen receptors (CARs).

### Pharmaceutical Composition

The cells of the present invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy product is preferably performed in compliance with FACT-JACIE International Standards for cellular therapy.

Haematopoietic Cell, Haematopoietic Stem and/or Haematopoietic Progenitor Cell Transplantation

The present invention provides a population of haematopoietic cells, haematopoietic stem cells and/or haematopoietic progenitor cells, prepared according to a method of the invention for use in therapy, for example for use in gene therapy.

The use may be as part of a cell transplantation procedure, for example a haematopoietic stem cell transplantation procedure.

Haematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In one embodiment, a population of haematopoietic stem cells prepared according to a method of the invention is administered as part of an autologous stem cell transplant procedure.

In another embodiment, a population of haematopoietic stem cells prepared according to a method of the invention is administered as part of an allogeneic stem cell transplant procedure.

The term “autologous stem cell transplant procedure” as used herein refers to a procedure in which the starting population of cells (which are then transduced according to a method of the invention) is obtained from the same subject as that to which the transduced cell population is administered. Autologous transplant procedures are advantageous as they avoid problems associated with immunological incompatibility and are available to subjects irrespective of the availability of a genetically matched donor.

The term “allogeneic stem cell transplant procedure” as used herein refers to a procedure in which the starting population of cells (which are then transduced according to a method of the invention) is obtained from a different subject as that to which the transduced cell population is administered. Preferably, the donor will be genetically matched to the subject to which the cells are administered to minimise the risk of immunological incompatibility.

Suitable doses of transduced cell populations are such as to be therapeutically and/or prophylactically effective. The dose to be administered may depend on the subject and condition to be treated, and may be readily determined by a skilled person.

Haematopoietic progenitor cells provide short term engraftment. Accordingly, gene therapy by administering transduced haematopoietic progenitor cells would provide a non-permanent effect in the subject. For example, the effect may be limited to 1-6 months following administration of the transduced haematopoietic progenitor cells.

Such haematopoietic progenitor cell gene therapy may be suited to treatment of acquired disorders, for example cancer, where time-limited expression of a (potentially toxic) anti-cancer nucleotide of interest may be sufficient to eradicate the disease.

The invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated of receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the invention may be useful in the treatment of β-thalassemia, chronic granulomatous disease, metachromatic leukodystrophy, mucopolysaccharidoses disorders and other lysosomal storage disorders.

As mentioned above, the applicability of the invention to T cells also facilitates its application in cell therapies that are based on infusion of modified T cells into patients, including anti-cancer strategies (such as using engineered CAR-T cells) and approaches based on infusion of universal donor T cells. Thus, in addition, or in the alternative, the invention may be useful in the prevention of graft-versus-host disease.

### Method of Treatment

It is to be appreciated that all references herein to treatment include curative, palliative and prophylactic treatment; although in the context of the invention references to preventing are more commonly associated with prophylactic treatment. In one embodiment, the treatment of mammals, particularly humans, is preferred. Both human and veterinary treatments are within the scope of the invention.

The skilled person will understand that they can combine all features of the invention disclosed herein without departing from the scope of the invention as disclosed.

### Administration

Although the agents for use in the invention (in particular, the populations of cells produced by a method of the invention) can be administered alone, they will generally be administered in admixture with a pharmaceutical carrier, excipient or diluent, particularly for human therapy.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

A “subject” refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1-AAV ITRs are the Main Culprit of p53 Pathway Activation Via Mre 11-Rad50-Nbs1 (MRN) Complex in Edited HSPCs

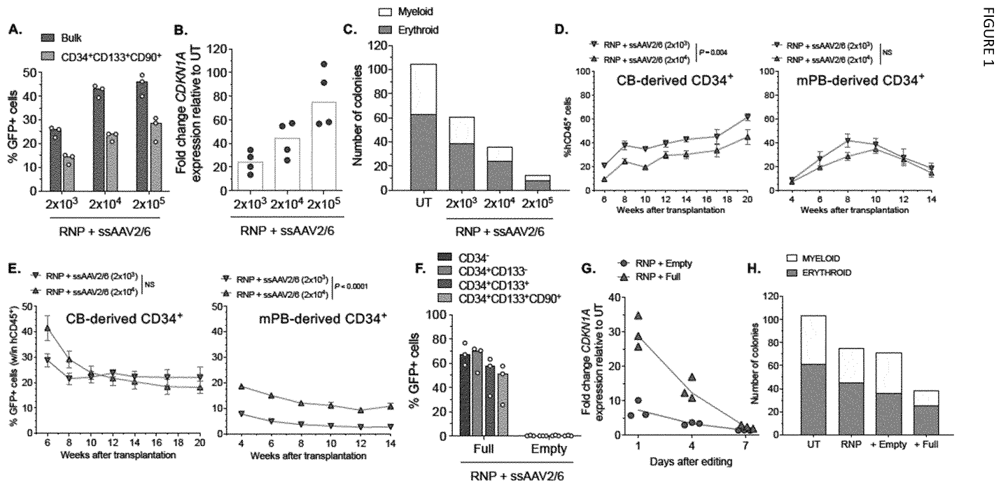

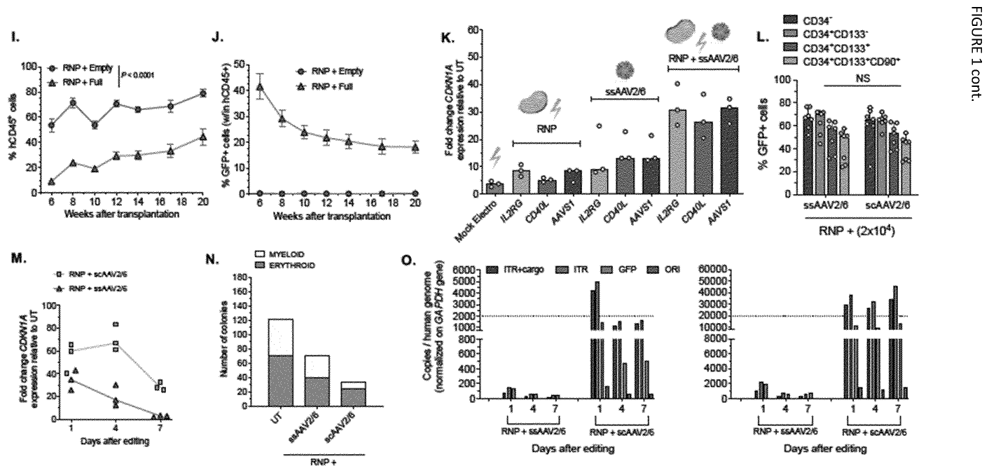

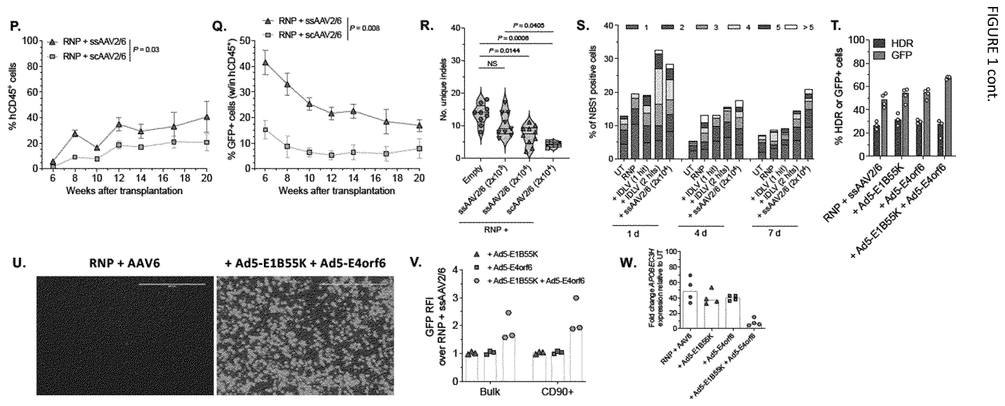

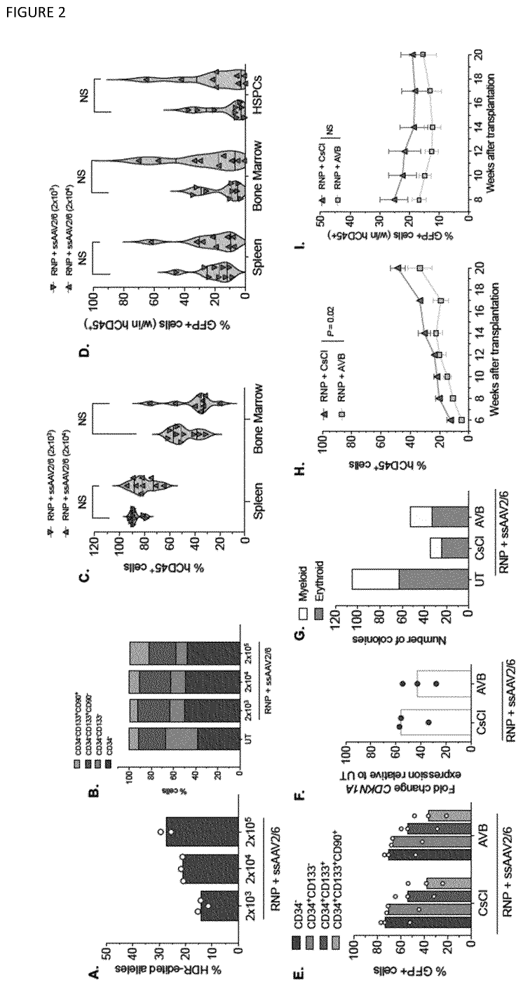

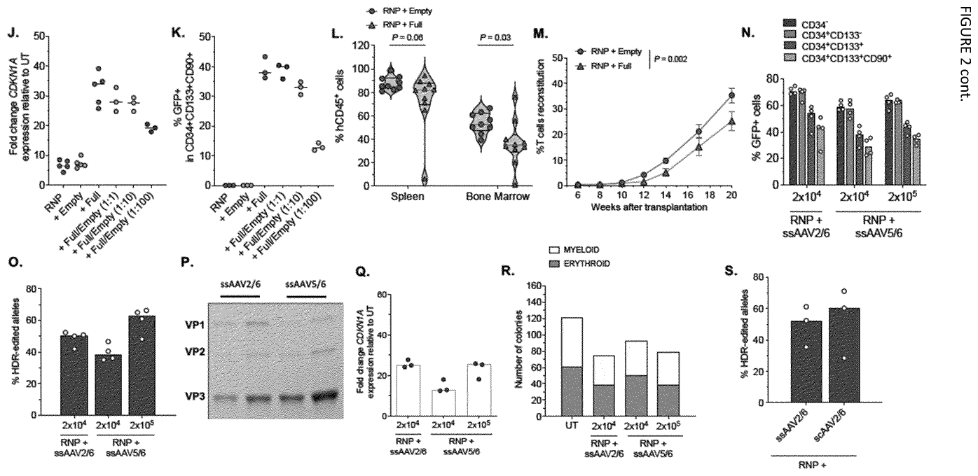

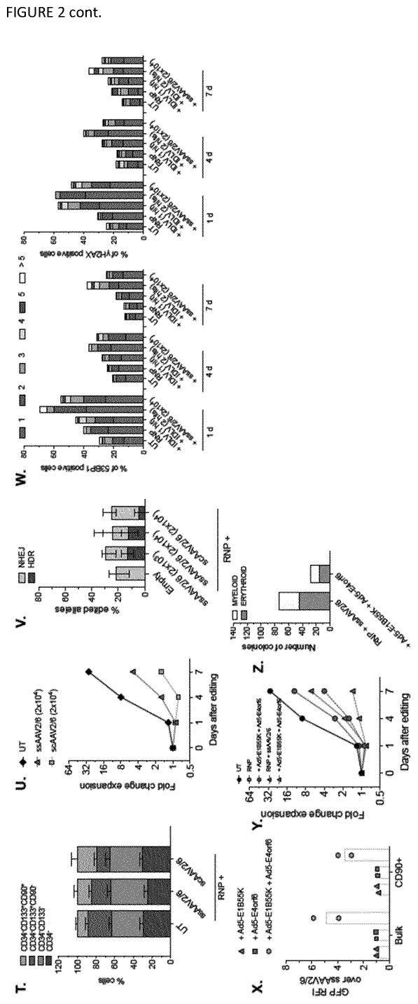

We performed HDR-based gene editing by targeting the AAVS1 safe harbor locus. We electroporated umbilical cord-blood (CB) HSPCs with a previously validated and highly specific RNA guide (sgRNA) delivered as ribonucleoprotein (RNP) with S.p. Cas9 nuclease (Schiroli, et al., Cell Stem Cell 24, 551-565.e8, 2019). Upon electroporation cells were transduced with a single strand (ss) AAV2/6 purified by cesium chloride (CsCl) density gradient, which carried homologies for AAVS1 locus and an eGFP reporter sequence under transcriptional control of the human phosphoglycerate kinase (PGK) promoter. By measuring the percentage of edited cells and the DDR-dependent CDKN1A (p21) induction, we found that incremental doses of ssAAV2/6 progressively increased editing efficiencies in the bulk and the most primitive HSPCs (phenotypically defined as CD34⁺ CD133⁺ CD90⁺) at the expense of an exacerbated p53 activation (FIGS. 1a, b and FIGS. 2a, b) and reduced in vitro clonogenic potential (FIG. 1c). To investigate in vivo engraftment capacity of edited HSPCs, we transplanted immunodeficient NOD.Cg-Prkdc^{scid} II2rg^{tm1Wl}/SzJ (NSG) mice with a non-saturating dose of CB or mobilized peripheral blood (mPB) HSPCs edited with the lowest (2×10³ vg/cell) or intermediate (2×10⁴ vg/cell) ssAAV2/6 doses, which allowed efficient HDR editing with moderate toxicity in vitro. The lowest dose resulted in better repopulation than the intermediate one in peripheral blood (PB) and hematopoietic organs (FIG. 1d, FIG. 2c). The proportion of edited (GFP⁺) cells in the graft was higher during the early phase of hematopoietic reconstitution for the “intermediate dose” groups, likely reflecting higher permissiveness to HDR-editing of short-term repopulating cells. While the AAV dose response for edited cells was more evident for mobilized PB sourced cells, the fraction of edited cells was higher for CB-sourced cells, likely due to their higher susceptibility to transduction (FIG. 1e, FIG. 2d). Purification of ssAAV2/6 by AVB Sepharose immune-affinity chromatography, a clinically relevant methodology, did not improve the editing protocol (FIG. 2e-i).

To further investigate whether the capsid and/or the viral DNA of ssAAV2/6 particles is responsible for p53 triggering in HSPCs, we edited AAVS1 in presence of “empty” or “full” AAV6-derived capsids (as fractionated by CsCl gradient) (FIG. 1f). Treatment with ssAAV2/6 full particles triggered a robust and transient p21 induction decreasing HSPC clonogenic potential. Treatment with empty AAV particles showed much less p21 induction and preserved clonogenic capacity comparable to RNP-only treated cells (FIG. 1g, h). Indeed, adding increasing amounts of empty particles to full ones progressively decreased p21 induction and editing efficiency, likely due to competition for cellular transduction (FIG. 2j, k). HSPCs edited with ssAAV full particles showed 2-to-3-fold lower engraftment in vivo than cells treated with empty vector both in the blood and hematopoietic organs (FIG. 1i, j and FIG. 2l) as well as delayed T cell reconstitution (FIG. 2m). These findings uncover the encapsidated AAV genome as the culprit of p53 activation in HSPCs. Editing multiple sites (IL2RG, CD40L, AAVS1), with different AAV constructs that share the same AAV2 ITRs but different cargos showed similar p21 induction, which is cumulative with that induced by RNP, thus pointing to the AAV ITR as responsible for DDR activation (FIG. 1k). To exclude that p53-mediated toxicity is a specific feature of the AAV2 ITR sequence, we performed editing with ssAAV5/6 whose ITR sequences evolutionary diverged along the AAV phylogenic tree and contain fewer putative p53 binding sites. ssAAV5/6 allowed HDR editing in all HSPC subpopulations, albeit lower than ssAAV2/6 at matching dose, possibly due to the decreased infectivity resulting from lower content of viral protein 1 (VP1) in the capsid (FIG. 2n-p). However, at comparable levels of editing efficiency, ssAAV5/6 resulted in p21 induction and reduced clonogenic potential similar to ssAAV2/6 (FIG. 2q, r), suggesting that common structural features of AAV ITRs rather than serotype-specific sequences are responsible for DDR signaling in HSPCs.

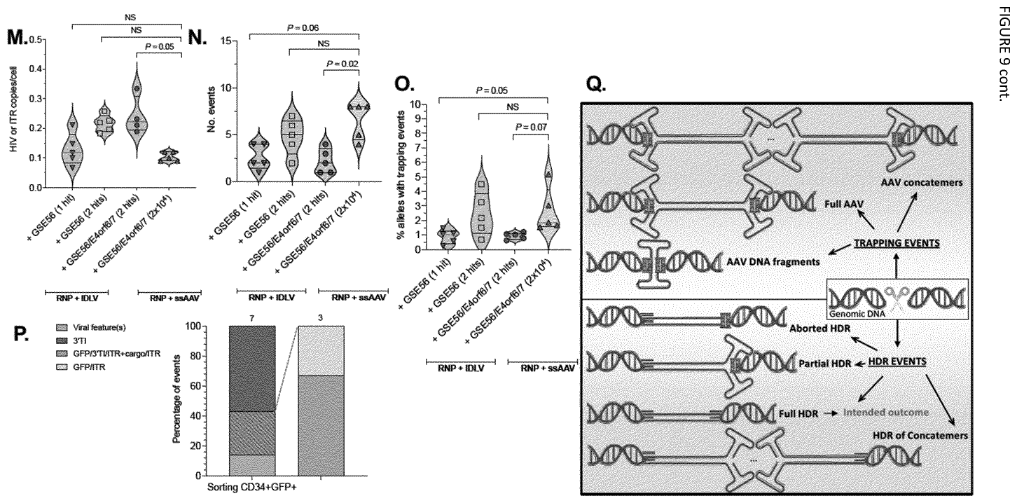

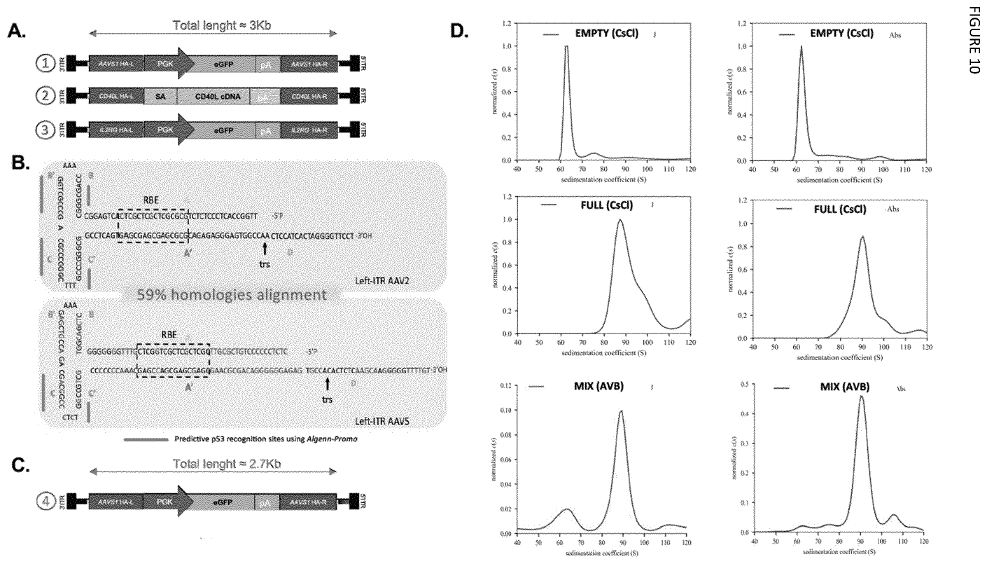

We then compared ss- with self-complementary (sc) AAV2/6, which is reported to be encapsidated as double stranded genome folded at an intervening ITR sequence and found equal editing efficiencies among subpopulations in culture (FIG. 1l and FIG. 2s, t). Unexpectedly, scAAV2/6 further exacerbated and prolonged p21 induction while drastically impairing cell growth and in vitro clonogenicity, despite comparable purity/yield of the batches tested (FIGS. 1m, n and FIG. 2u). Quantification of AAV DNA, using different set of probes along the genome (“GFP”, “AAV ITR+cargo” and “ITR” ì ddPCR assays) showed substantial and persistent amounts of AAV genomes in the treated HSPCs, consistent with robust induction of DDR which, in turn, by halting cell proliferation, may prevent effective dilution of the episomal DNA (FIG. 1o). Notably, AAV copies per human genome (CG) were on average 10-fold higher and more persistent for scAAV2/6 than ssAAV2/6, again in line with increased toxicity. Concordantly, HSPC transduction with scAAV2/6 significantly reduced engraftment capacity and almost abrogated the repopulating potential of HDR-edited cells (FIG. 1p, q). The frequency and diversity of insertions/deletions (indels) generated at the target site by NHEJ can be used as surrogate readout of graft complexity. Targeted deep sequencing of AAVS1 in hematopoietic organs of xenotransplanted mice showed that full ssAAV2/6 particles decreased indels diversity in a dose-dependent manner, which was further exacerbated when using scAAV2/6 (FIG. 1r and FIG. 2v). Furthermore, we detected the presence and persistence of DNA sequences of AAV plasmid E. coli replication origin (“ORI” ddPCR assay) (see FIG. 10), although at 10-to-30-fold lower abundance than the AAV genome, likely due to reverse packaging of portions of the plasmid backbone in the viral particle.

Several studies showed that the MRN complex, which binds to free DNA ends and engages the repair machinery binds the AAV ITR and inhibits transgene expression in transduced cells. We then performed immunofluorescence staining against Nbs1, a subunit of the MRN complex. Nbs1 foci were increased in nuclease-treated cells, and further exacerbated in presence of the ssAAV2/6 template, even several days after editing (FIG. 1s). An increase in foci formation was also observed staining for the DDR sensor/marker 53BP1 and γH2AX, following a similar pattern over time as seen for Nbs1 (FIG. 2w). Note that ex vivo HSPC culture triggered the formation of some foci in control untreated cells (UT), probably due to replication stress in culture. We then induced transient expression of adenoviral proteins Ad5-E1B55K and Ad5-E4orf6 during editing, whose combination was shown to inhibit MRN complex activity by promoting Mre11 degradation. Molecular analysis showed similar HDR efficiencies in presence or absence of these Ad proteins (FIG. 1t). However, the fraction of positive cells and the level of expression of GFP were higher upon editing in presence of the Ad proteins (FIGS. 1u, v and FIG. 2x), in agreement with previous reports in other cell types (Chu et al., 2015, Nature Biotechnology, 33:543-548). Notably, we observed nearly abrogated induction of the p53 downstream effectors APOBEC3H (FIGS. 1w) and p21, highlighting a central role of MRN in triggering the AAV-dependent DDR. Note that in vitro HSPC expansion and clonogenicity were drastically affected by this treatment, suggesting that MRN inhibition is detrimental for cell proliferation when blocking, even transiently, DNA repair (FIGS. 2y, z).

Overall, these results point out a central role of AAV ITRs in triggering a p53 response via MRN complex and, consequently, lead to a reduction of the human graft size in xenotransplantation settings.

### Example 2-Trapping of Transcriptionally Active AAV ITR Sequences at the Target Site is an Inadvertent Consequence of HDR Editing in HSPCs

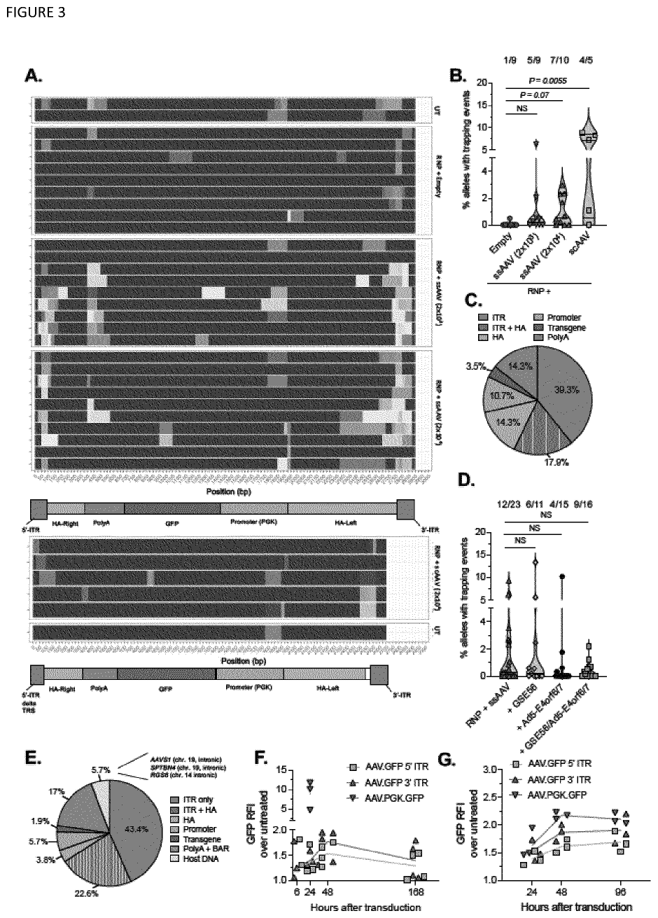

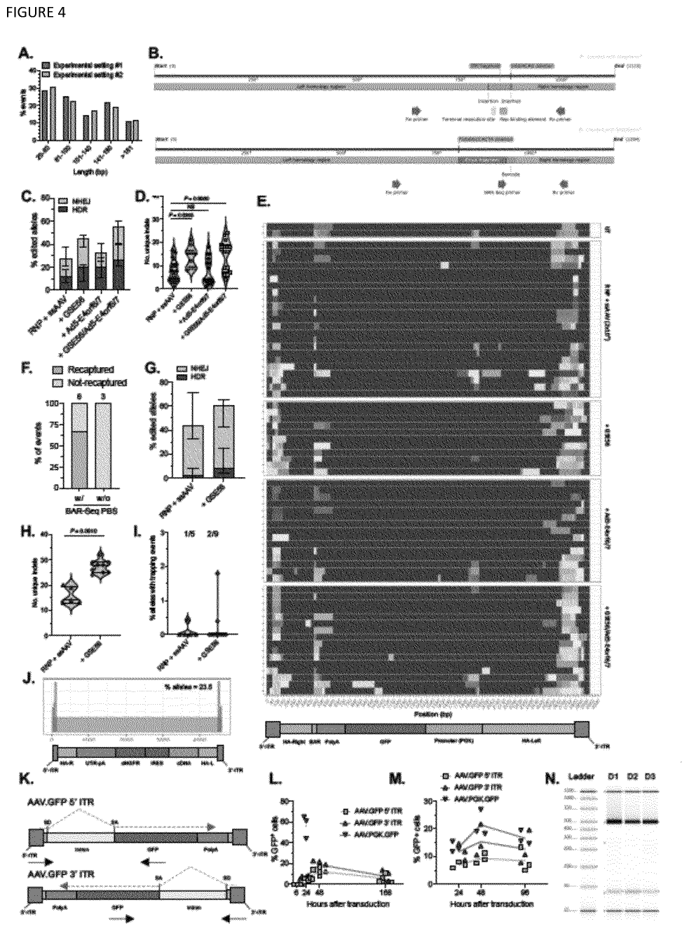

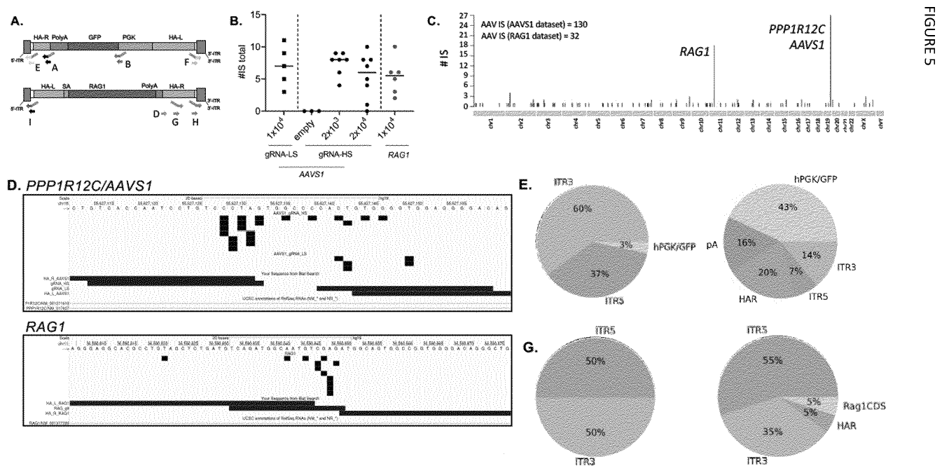

Granular inspection of reads alignments from the indels analysis at the edited AAVS1 reported above highlighted the presence of alleles carrying insertions of AAV sequences with variable lengths (up to 210 bp; FIG. 3a and experimental setting no. 1 in FIG. 4a) in about two thirds of mice receiving HSPCs edited with full AAV2/6 particles (FIG. 3b). AAV-containing alleles ranged in frequency from the lower threshold of detection (set at 0.2%) to 3% of the total allele diversity in most mice, except for 4 out of 33, which showed much higher abundance. Three of these mice belonged to the scAAV group, which having the lowest graft clonality may show relative overrepresentation of any contributing clone. We then extracted the inserted DNA sequences and found that >55% aligned to AAV ITR sequences (FIG. 3a, c), comprising the D sequence, the terminal resolution site (trs), the rep-binding element and a portion of the palindromic sequences within the ITR which form a T-shaped structure (FIG. 4b). In a subfraction of ITR-containing sequences, the distal portion of the homology arms was also included and allowed to identify prevalence of the 3′ ITR in these trapping events. Interestingly, we often observed deletion of some nucleotides flanking the insertion site, reminiscent of NHEJ-induced indels (FIG. 4b, top). A minor subset of insertion events mapped inside the transgene cassette with a prevalence of those sequences immediately adjacent to the proximal portion of the homology arms (polyA and human PGK promoter), possibly reflecting aborted HDR events (FIG. 3a, c).

To consolidate these observations and evaluate whether the use of HDR editing enhancers could influence the frequency or the pattern of AAV fragments integrations at the target site, we performed an extensive analysis on 65 samples from the long-term xenograft of mice belonging to four previously published experiments (Ferrari et al., 2020, Nature Biotechnology). The frequency and number of unique NHEJ-edited alleles in the human graft were higher in groups treated with the p53 inhibitor GSE56 both in presence and absence of Ad5-E4orf6/7, confirming the previously observed increased clonal composition of the edited graft upon transient p53 inhibition (FIG. 4c, d). As above, we found integration of AAV DNA fragments of variable lengths at the nuclease target site (experimental setting no. 2 in FIG. 4a). No obvious differences were found among groups in terms of fraction of mice carrying at least one event and median allele frequency for each mouse (˜0.5%), with some animals showing >2% of alleles carrying trapping of AAV fragments (FIG. 3d). Moreover, the distribution of events upon alignment to the reference AAV genome was similar among groups, with prevalence of ITR-containing sequences (˜65% of all events) (FIG. 3e and FIG. 4e). Because an AAV donor carrying a barcoded sequence downstream the transgene cassette and upstream the homology arm (on the left in the drawing of FIG. 4e) was used for clonal tracking of edited cells in these set of experiments, we verified whether barcodes (BARs) retrieved as insertions in the target site had also been recovered in the previous clonal tracking analysis. This was true for most barcoded AAV fragments long enough to comprise the primer binding site used for BAR-Seq NGS, providing an independent confirmation of their occurrence in the mouse graft (FIG. 4f). Moreover, sequencing of both junctions of these insertions provided additional support to our hypothesis that they originated from NHEJ on one side and aborted HDR on the other one (FIG. 4b, bottom). Finally, we also found three trapping events, which carried DNA sequences aligning to the human cellular genome, two of which mapped to the q-arm of chromosome 19 (the same of AAVS1) (FIG. 3e).

To exclude an influence of the targeted locus and cell type on AAV fragments trapping, we performed the same analyses in two previously published experiments (Vavassori et al., 2021, EMBO Molecular Medicine, 13: e13545) based on xenotransplantation of CD40LG-edited human HSPCs (FIG. 4g, h) or T cells, which confirmed the integration of DNA fragments of AAV origin in both cell types (FIG. 4i, j).

Putative transcriptional activity of AAV ITRs might be of concern upon integration of full-length or partial sequences in the human cell genome. To assess whether AAV2 ITRs have promoter activity in human hematopoietic cells, we designed an AAV2/6 carrying the GFP transgene downstream of either the 5′ or the 3′ ITR (FIG. 4k). Transduced human primary hematopoietic cells from multiple donors showed detectable GFP protein expression peaking at 48 hours after transduction and progressively decreasing over time (FIG. 3f, g and FIG. 41, m). Of note, transduction with the 3′ ITR reporter vector resulted in a slightly higher fluorescence intensity and percentage of GFP⁺ cells than the 5′ ITR reporter one, concordantly with previous reports in other contexts. Successful amplification of the spliced GFP transcript confirmed the presence of transcriptional start site(s) upstream of the splicing donor site and therefore within the ITR sequence (FIG. 4n).

Overall, these findings reveal unanticipated consequences that may aggravate the genotoxic burden of AAV-based HDR editing in primary hematopoietic cells, including LT-HSPCs.

### Example 3-Unbiased Genome-Wide Integration Site Analysis Reveals AAV DNA Inserted at Nuclease On- and Off-Target Sites in LT-HSCs

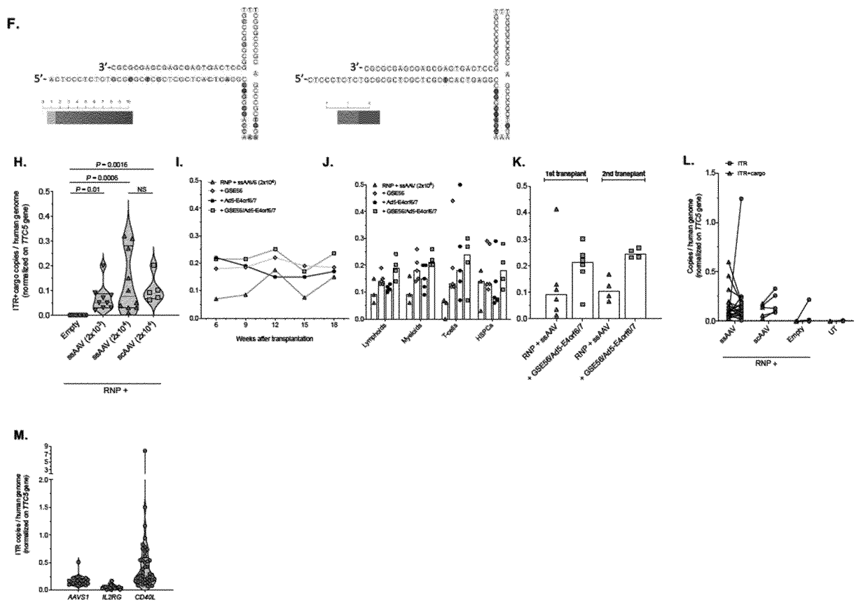

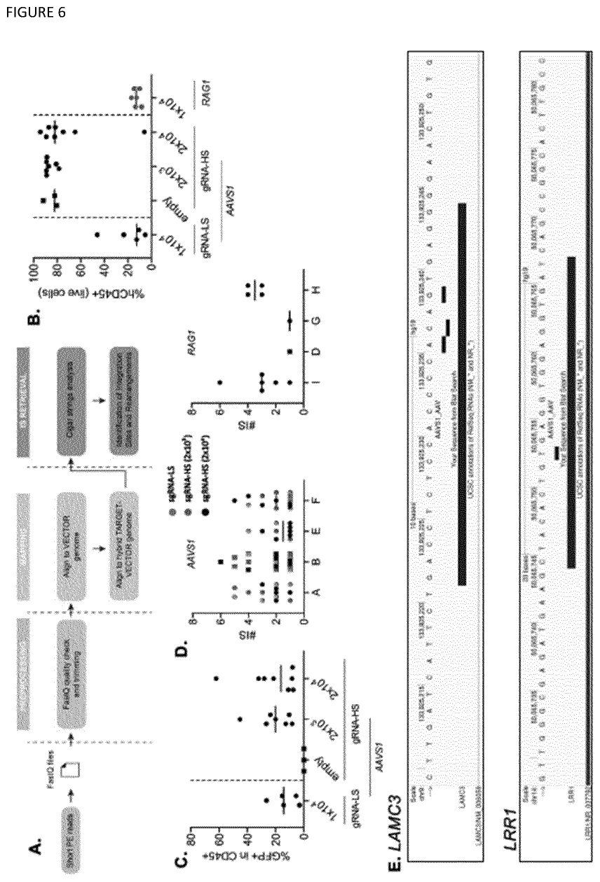

To assess the eventual occurrence of AAV integration outside of the editing locus, we developed a methodology for unbiased, genome-wide retrieval and identification of AAV integration sites (IS). We adapted a nested PCR protocol for AAV IS retrieval following random shearing of genomic DNA (Sonication Linker-Mediated, SLIM; see Methods), which we and others have extensively used for clonal tracking and safety studies in patients treated with LV-based HSPC gene therapy, whose reliability and sensitivity was recently described (Cesana et al., 2021, Nature medicine, 27:1458-1470). Since AAV DNA may integrate as fragments as shown here and in other studies, several primers were designed to amplify AAV-cellular genome junctions involving different locations of the AAV genome (FIG. 5a, top). Amplicon libraries were assembled, sequenced and analyzed by an ad-hoc bioinformatics pipeline (Recombinant Adeno-Associated Vector Integration analysis, RAAVIoli), which filtered sequencing reads for quality, removed adapters and linker sequences added during the amplification steps and aligned the remaining portion against the AAV and human genomes. AAV IS were identified only from sequencing reads containing both AAV and human genomic DNA sequences (see Methods for details on breakpoint identification and filtering criteria for inclusion/exclusion of IS, FIG. 6a).

We analyzed genomic DNA samples collected from the bone marrow and spleen of 23 mice transplanted with CB CD34⁺ cells edited at AAVS1 with either a high (HS, see FIG. 1i) or low (LS) specificity sgRNA (FIG. 6b, c) and empty or full ssAAV. After collapsing all chimeric AAV/human reads containing the same junction, we identified 130 non-redundant IS, ranging from 1 to 11 unique IS per mouse, while no IS were retrieved from the empty AAV group (FIG. 5b). No significant differences were observed in the number of IS identified from all other treatment groups, whether differing for the AAV dose or guide, and among the different PCR systems used for the amplification step (FIG. 5b and FIG. 6d, left). The editing site within PPP1R12C, the AAVS1 host gene, was the preferential location for integration accounting for 21% (N=27) of the identified IS (FIG. 5c and Table 1). These IS tightly clustered into the protospacer sequence targeted by the two different sgRNAs used, proving that AAV integration was promoted by the nuclease-induced DSB (FIG. 5d, top). Interestingly, among the other IS identified at lower frequency, two of them, LAMC3, which was the third ranking IS, and LRR1, were also captured by in silico (CRISPOR) and GUIDE-Seq specificity analyses performed for the LS sgRNA (Table 2, 3). AAV integrations at these off-target sites occurred specifically within the genomic regions homologous to the sgRNA, proving their origin from an off-target activity of the RNP (FIG. 6e). In addition, 3 independent IS mapped to the exon 1 and intron 1 of the PGK gene (FIG. 6f), suggesting that the homology between the vector-contained and cellular PGK promoter sequence might favor recombination at this transcriptionally active locus. Of note, these latter IS were amplified using the PCR set whose primers anneal to a vector-specific sequence at the 3′ end of the PGK promoter and the amplified downstream cellular sequences were not comprised within the vector, making unlikely that this sequence originated from a technical artifact in their retrieval. Remaining IS may represent insertions at random spontaneous DNA breaks or at additional unpredicted off-targets.

TABLE 1
Genes targeted by AAV and IDLV integration.

| | Chr | GeneID | N IS | N mice | % | PCR | Off-Target |
|---|---|---|---|---|---|---|---|
| AAVS1_AAV | 19 | PPP1R12C | 27 | 12 | 20.8 | B/E/F | |
| | 2 | LINC00486 | 4 | 3 | 3.1 | E/F | |
| | 9 | LAMC3 | 3 | 2 | 2.3 | E/F | LS |
| | X | PGK | 3 | 3 | 2.3 | B | |
| | X | CD40LG | 2 | 2 | 1.5 | A | |
| | 7 | SLC4A2 | 2 | 2 | 1.5 | B | |
| | 3 | ERC2 | 2 | 1 | 1.5 | F | |
| | 9 | MOB3B | 2 | 1 | 1.5 | F | |
| | 15 | ISG20 | 2 | 1 | 1.5 | E | |
| | 20 | PANK2 | 2 | 1 | 1.5 | F | |
| | 14 | LRR1 | 1 | 1 | 0.8 | F | LS |
| RAG1_AAV | 11 | RAG1 | 18* | 5 | 56.3 | I/H | |
| | 2 | SCG2 | 2 | 1 | 6.3 | I | |
| | 12 | PPFIA2 | 2 | 1 | 6.3 | I | |
| | 17 | TIMM22 | 2 | 1 | 6.3 | H | |
| AAVS1_IDLV | 9 | LAMC3 | 6 | 3 | 11.3 | B/L | LS |
| | 19 | PPP1R12C | 5 | 3 | 9.4 | B/L | |
| | X | PGK1 | 3 | 3 | 5.7 | B | |
| | 1 | DISP3 | 2 | 2 | 3.8 | B | |
| | 11 | OR4C46 | 4 | 1 | 7.5 | L | |
| | 7 | LINC00972 | 2 | 1 | 3.8 | L | |
| | 22 | SYN3 | 2 | 1 | 3.8 | L | |
| | 22 | ZDHHC8 | 1 | 1 | 1.9 | L | LS |

TABLE 2
Specificity analysis for AAVS1-LS sgRNA.

| | | | | | | GUIDE-Seq | |
|---|---|---|---|---|---|---|---|
| | | | CRISPOR specificity output | | | specificity output | |
| | | Target | MIT | Number | OTs for | Number | OT for |
| Guide | Target | sequence | specificity | of OTs | 0-1-2-3-4 | of OTs | 0-1-2-3-4 |
| ID | locus | (5′-3′) | score | predicted | mismatches | identified | mismatches |
| AAVS1- | PPP12RC, | GTCACCA | 46 | 342 | 0-1-7- | 0 | 0-0-5-2- |
| LS | intron | ATCCTGT | | | 41-293 | | 2 |
| | 1 | CCCTAGT | | | | | |
| | | GG | | | | | |

TABLE 3
List of OTs for the AAVS1-LS sgRNA identified by GUIDE-Seq.

| | | GUIDE- | | | | |
|---|---|---|---|---|---|---|
| Mismatch | Mismatch | Seq peak | | | | |
| position | count | score | Chr | Start | End | Gene ID |
| T . . . A . . . CA | 2 | 67 | 14 | 34255202 | 34255220 | NPAS3 |
| | | | | | | (intron) |
| . . . TA . . . | 4 | 60 | 14 | 85911475 | 85911495 | LINC00911 |
| | | | | | | (intergenic) |
| A . . . A . . . T . . . | 3 | 293 | 17 | 18869239 | 18869255 | SLC5A10 |
| | | | | | | (intron) |
| . . . G . . . C . . . CA | 4 | 72 | 19 | 3063352 | 3063372 | TLE5 |
| | | | | | | (intergenic) |
| A . . . A . . . | 2 | 1184 | 19 | 18608984 | 18609001 | ELL |
| | | | | | | (intron) |
| AG . . . | 2 | 113 | 3 | 38031626 | 38031646 | VILL |
| | | | | | | (intron) |
| . . . A . . . | 1 | 1633 | 4 | 108975643 | 108975657 | LEF1 |
| | | | | | | (intron) |
| . . . A . . . T . . . | 2 | 17135 | 9 | 133925231 | 133925245 | LAMC3 |
| | | | | | | (intron) |
| . . . T . . . C . . . A . . . | 3 | 54 | X | 49066556 | 49066574 | CACNA1F |
| | | | | | | (intron) |

We then investigated what regions of the AAV genome were involved in integration events. Overall, 97% of integrations at AAVS1 and two predicted off-target sites involved ITRs (FIG. 5e, left). Consistently with previous results and other reports, the 3′ITR was prevalent over the 5′ITR in these events. Outside the editing on- and off-target sites, 43% of IS involved vector-derived PGK promoter sequences, 21% the ITRs and the remaining subset of IS involved the right homology arm and the polyA site (FIG. 5e, right). A more granular inspection identified some preferred nucleotide breakpoints within the “A” and “C” loop regions of 3′ITR element (FIG. 5f, left), as previously reported (Nguyen et al., 2020, Nature Biotechnology, 39 (1): 47-55).

Since PCR steps are performed after sonication of the genomic DNA, we can rank their clonal abundance by measuring the number of sequencing reads of different lengths containing the same genomic junction. More than half of AAV-containing clones were represented by >5 to nearly 100 genomes, implying their proliferation and relevant contribution to the graft (FIG. 6g). A similar analysis was performed on 6 mice repopulated with bone marrow-derived HSPCs edited at the RAG1 gene, within the intron 1 (FIG. 5a, bottom and FIG. 6b, h). The repair template was a promoter-less codon-optimized RAG1 sequence downstream of a splice acceptor site and was delivered by ssAAV, modeling correction of mutant defective alleles causing primary immunodeficiency. The RAAVIoli platform identified 32 unique AAV IS, 12 of which (38%) were trapped at the RAG1 editing site (FIG. 5b, c, FIG. 5d, bottom, and FIG. 6d, right, and 6g). As for AAVS1 editing, ITR fragments were the most frequent portion of AAV DNA inserted at on- and off-target sites with similar preferences for the nucleotide breakpoint (FIG. 5f, right and 5g)

Overall, these analyses identified reproducible and significant occurrence of integration of AAV DNA at DSB induced by editing at on and off nuclease target sites, mainly involving a specific region of the ITR, which might trigger or be prone to capture during NHEJ-mediated repair.

### Example 4-Quantification of AAV DNA Trapping Reveals an Unexpectedly High Frequency of Integration in Edited Long-Term Engrafted HSPCs

Since short-read sequencing of the edited locus and genome-wide IS identification may underestimate the overall frequency of trapping events, we performed ddPCR analyses to get an independent genome-wide quantification of AAV genome trapping events. Long-term human xenografts from FIG. 1 showed detectable signal when probing for an AAV sequence spanning from the ITR trs to the homology arm (ITR+cargo) in nearly all mice with median 0.05 or 0.1 CG depending on the group. This pattern was notably higher than the frequency estimated from the target site sequencing and was similar for the ssAAV2/6 and scAAV2/6 groups and null in the “empty AAV” (FIG. 5h). On the contrary, we detected only background noise signal when probing for the AAV plasmid contaminants in the human long-term graft (FIG. 6i). To increase the number of replicates and evaluate the impact of editing enhancers, we performed the ITR+cargo assay on the long-term xenograft of mice transplanted with AAVS1-edited HSPCs in previously published experiments (Ferrari et al., 2020, Nature Biotechnology) and found more copies in presence of GSE56 and/or Ad5-E4orf6/7 (FIG. 6j). After pooling an even larger cohort of mice, we found median ITR+cargo CG of 0.08 and 0.18 (FIG. 6k) in the long-term graft of mice showing median 13 and 25% of cells edited with standard and optimized protocols, respectively. Of note, ITR+cargo CG were stable over time and across lineages, including BM-derived CD34⁺ HSPCs (FIG. 5i, j). Furthermore, we confirmed similar ITR+cargo CG in the human long-term graft of primary and secondary recipients as well as in human CD34⁺ HSPCs of secondary hosts, with more copies maintained in the enhanced editing group (FIGS. 5k and 61). Altogether, these results conceivably rule out that the ITR+cargo signal may come from carryover of episomal AAV genomes. Whether integration of AAV DNA occurs mostly as individual elements in a sizable fraction of cells treated for editing or as concatemers in only a small fraction of them cannot be determined by this analysis. Since ITR fragments are not detected by the ITR+cargo assay we performed ddPCR also with the ITR probe on PB samples of mice from FIG. 5g and found equal or higher signal in most mice (FIG. 51). The ITR signal in the “empty” group was comparable to the background threshold except for one mouse, possibly in agreement with the finding that fractionated empty AAV particles may carry ITR fragments at low abundance.

To verify whether measurable integration of AAV genome/fragments also occurs upon editing of other genomic sites of therapeutic relevance, we analyzed the long-term xenografts of mice transplanted with mPB HSPCs edited at CD40L or IL2RG. We found 1.5-fold higher and 1.5-fold lower median ITR CG, respectively, as compared to AAVS1-edited grafts (FIG. 5m), showing consistent occurrence but variable extent of AAV integration in cells edited at different target sites, likely dependent on the specificity and design of the sgRNA employed and/or the sequence, homology and content of the AAV genome.

### Example 5-Optimized IDLV Editing Shows a More Favorable Toxicity and Safety Profile than Aav and Reaches Higher Editing Efficiencies in LT-HSCs

Lentiviral vector (LV) transduction can be significantly enhanced by drugs favoring LV entry or relieving intracellular blocks. We previously reported that HSPC gene editing with IDLV as repair template also benefit by these optimizations, reaching HDR efficiencies close to those achieved by AAV-based HDR editing in CB HSPCs (Petrillo et al., 2018, Cell Stem Cell, 23:820-832.e9). IDLV delivery would also allow to increase cargo capacity for the HDR template and, most importantly, avoid transcriptional activity from viral DNA elements given the deletion of all known enhancer and promoter sequences in the self-inactivating long terminal repeats (SIN-LTRs) of commonly used LV (Zufferey et al., 1998, Journal of Virology, 72:9873-9880).

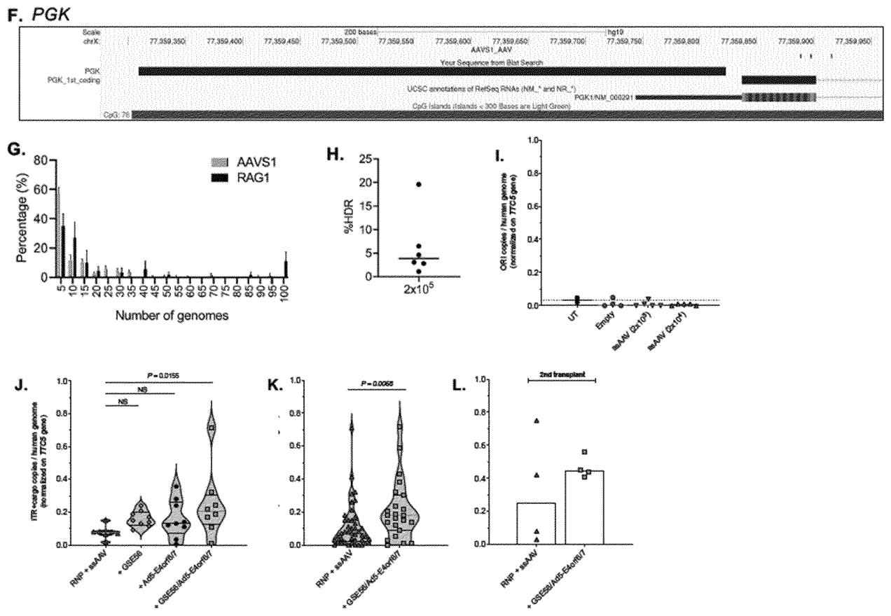

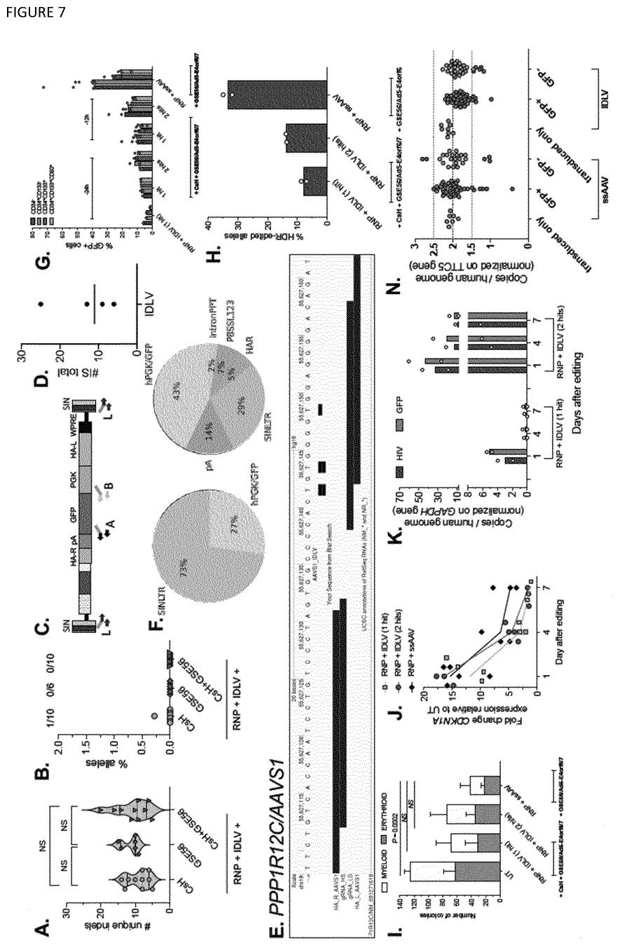

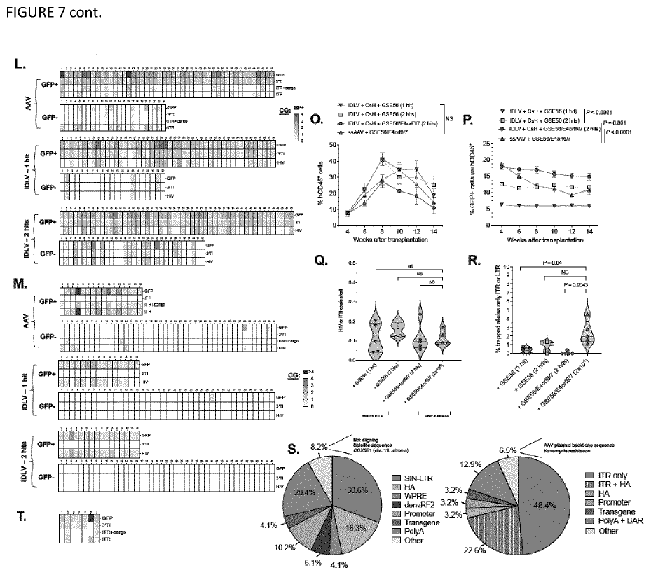

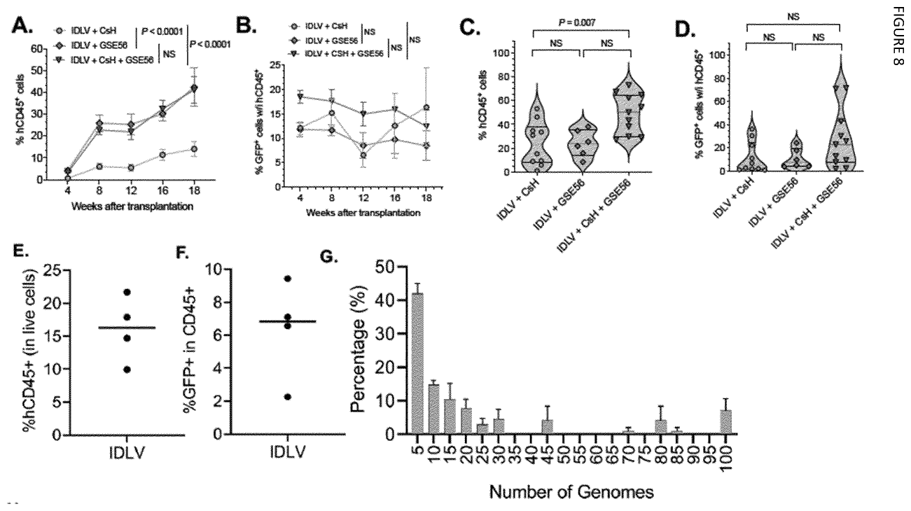

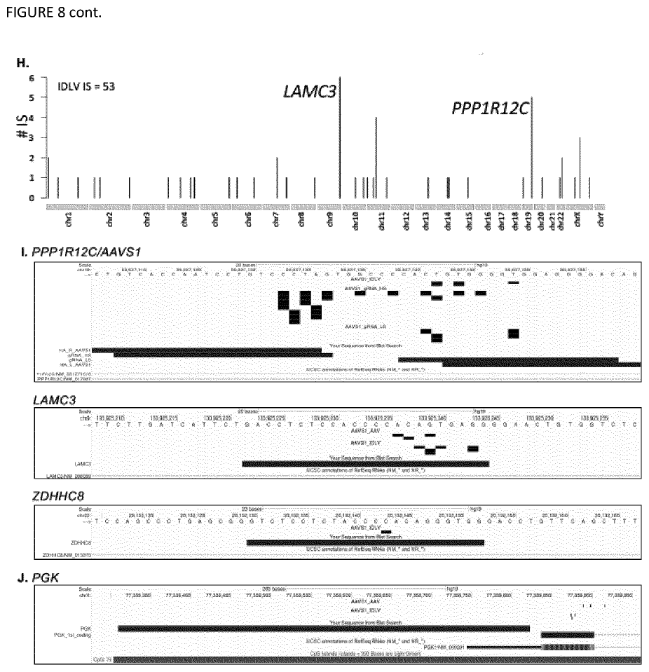

We thus performed two independent AAVS1 editing experiments in CB HSPCs using the cognate purified IDLV template in presence or absence of the p53 inhibitor GSE56 and the transduction enhancer cyclosporin H (CsH) previously described to overcome a potent LV entry block in human HSPCs (Petrillo et al., 2018, Cell Stem Cell, 23:820-832.e9). GSE56 in combination with CsH increased the size of the human graft and tended to increase the percentage of edited GFP⁺ cells among human circulating cells and in the bone marrow of xenotransplanted mice (FIG. 8a-d). Deep sequencing of the target locus in BM cells showed no differences among groups in term of indels diversity at NHEJ-edited sites (FIG. 7a), likely because the GSE56/CsH combination induced larger grafts also comprising larger fractions of HDR. Despite editing efficiencies were in the same range of AAV-based editing experiments, only one mouse out of 26 analyzed showed a single event of IDLV fragment integration with allelic frequency >0.2%, likely due to aborted HDR (FIG. 7b).

Next, we adapted the PCR protocols and the bioinformatics pipeline to screen for integration events of the IDLV donor template at genome-wide level (FIG. 7c). We analyzed DNA samples collected from the spleen of 4 long-term engrafted mice transplanted with CB HSPCs edited at AAVS1 with the LS sgRNA described above and IDLV as repair template (FIG. 8e, f). We identified 53 unique IS, ranging from 6 to 25 unique IS per mouse, all represented by multiple genomes (FIG. 7d and FIG. 8g). Also in this case, the editing site within the PPP1R12C gene was among the preferential integration site accounting for 9.6% (N=5) of the identified IS, all clustered into the LS sgRNA protospacer sequence (FIG. 7e and FIG. 8h, i). Moreover, 6 IS mapped to the same nuclease off-target sequence of LAMC3 that captured the AAV template in FIGS. 6e and 1 IS mapped to another predicted off-target site in ZDHH8 (Table 1 and FIG. 8i). Three IS were found in exon 1 of PGK (FIG. 8j), in agreement with the previously postulated recombination at the PGK locus because of homologous sequences contained within the vector genome. These findings confirm that IDLV can be captured at nuclease on and off target sites, similarly to what we described above for AAV, with preferential occurrence of the IDLV SIN LTR at the vector-genome junction (FIG. 7f). Such trapping, however, should mostly involve the whole genome or large fragments thereof, as our targeted deep sequencing analysis found a low occurrence of short IDLV derived sequences at the editing sites. Given the low frequency of LTR trapping and their transcriptionally inactive feature, we further tailored IDLV editing for the clinically relevant mPB HSPCs.

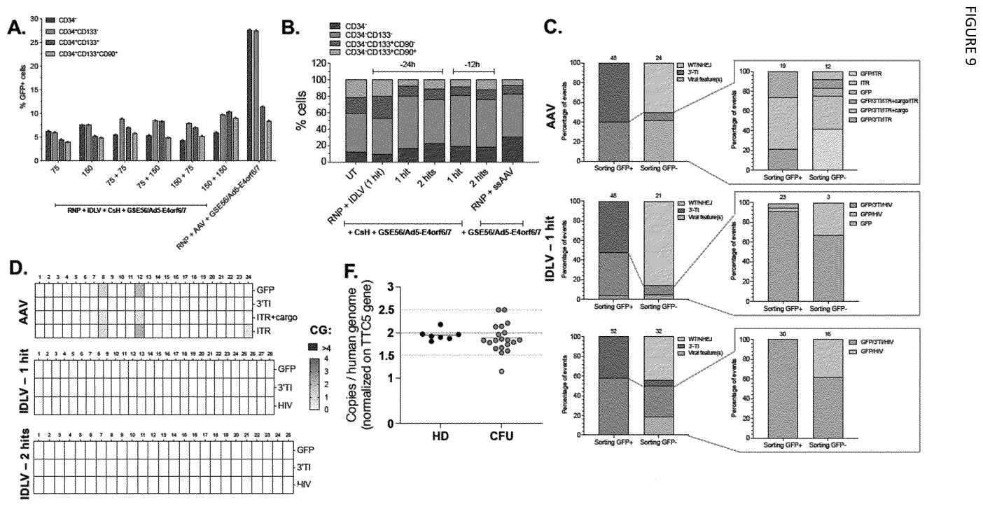

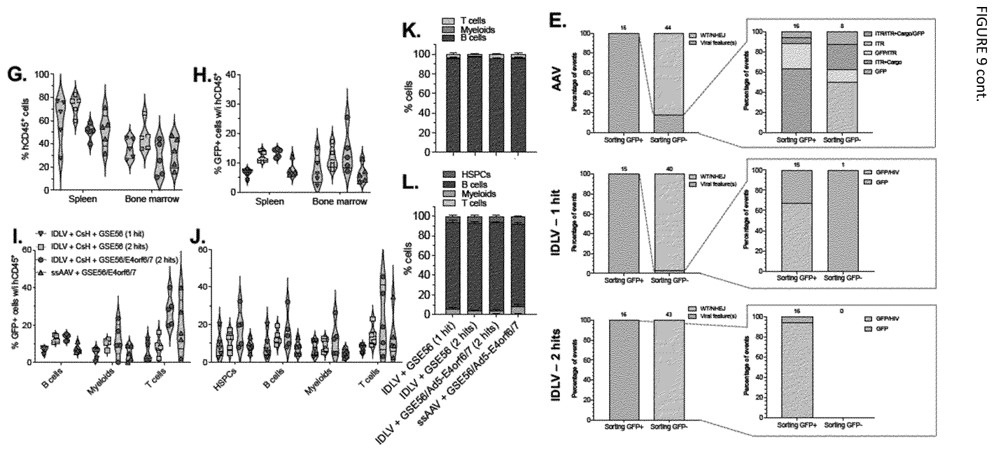

We thus performed AAVS1 editing in mPB HSPCs by combining CsH, GSE56 and Ad5-E4orf6/7. In order to identify the optimal conditions for maximal DNA donor availability during DSB repair, we tested different timings (12 or 24 hours before RNP electroporation) and one or two hits of IDLV delivery. The combination of all enhancers increased >8-fold the percentage of GFP⁺ cells in the most primitive HSPC compartment, which comprises LT-HSC. Two hits of transduction further increased editing efficiency in these cells up to 12% when using the highest MOIs (FIG. 7g and FIG. 9a). When comparing this optimized IDLV protocol with the AAV-based one we found that the latter was 3-times more efficient within committed progenitors (FIG. 7g), while the difference flattened in the most primitive compartment, explaining the higher overall percentage of HDR-edited alleles by AAV measured in the bulk culture (FIG. 7h). No differences in the proportion of most primitive HSPCs were found after editing with the optimized IDLV or AAV protocol (FIG. 9b). Notably, however, IDLV transduction better preserved HSPC clonogenic capacity, yielding 2-fold more colonies than the AAV-based protocol at matched cell input (FIG. 7i). This finding was consistent with a shorter wave of p21 induction after editing (FIG. 7j) and the substantially lower content and faster decay over time of intracellular IDLV DNA, as compared to AAV treatment (FIG. 7k and compare to matched experiment shown in FIG. 1o). Foci of DDR sensors, however, increased in similar manner in edited cells upon IDLV or AAV transduction (FIG. 1s).

We then performed a more granular assessment of the extent, preferential genomic features and possible underlying mechanisms leading to vector DNA integration for the two template delivery platforms. We screened with our panel of ddPCR probes tiling the vector genome, nearly 300 randomly picked colonies (CFU), outgrown from FACS-sorted GFP^{pos} or GFP^{neg} CB-derived HSPCs edited at AAVS1 with 70% efficiency for the AAV template and 28 or 38% efficiency for IDLV after one or two-hits, respectively. For both templates, nearly all GFP^{pos} colonies scored positive for both the payload (GFP) and the 3′TI assays (FIG. 7l and FIG. 9c). Among them, from 40 to 60%, according to the treatment group, carried putatively precise mono- or bi-allelic HDR mediated integration, given the absence of any signal for the assays probing for viral features (ITR+cargo and ITR for AAV, HIV for IDLV), while the remaining fractions tested positive also for one or more of them. These latter patterns suggest the occurrence of HDR on one junction and NHEJ on the other of the same edited allele, or, less likely, the occurrence of full HDR on one allele and NHEJ-mediated vector trapping on the other, or targeted integration of concatemers (with GFP probe >2), an outcome predominantly observed for AAV, or possibly concomitant off-target ITR/vector trapping. Analysis of colonies from sorted GFP^{neg} cells uncovered some GFP and 3′TI clones, which were more abundant for the two-hit IDLV protocol, likely due to delayed GFP expression after sorting (FIG. 71 and FIG. 9c). Notably, integration of viral DNA fragments in absence of GFP payload and 3′TI signal was reported exclusively for the AAV-based editing protocol and not for the IDLV one. Similarly, analysis of colonies plated from vector transduced-only HSPCs showed none of them carrying IDLV integration, while 3 out of 24 scored positive for at least one of the assays probing for AAV features (FIG. 9d), likely indicating integration of AAV DNA in spontaneous genomic DSBs.

To further evaluate the contribution of NHEJ-mediated trapping of full-length or fragmented viral vector genomes, independently from HDR, we transduced HSPCs with the same AAVS1 ssAAV2/6 or IDLV templates and edited them in the unrelated B2M locus, obtaining about 3% GFP⁺ cells for AAV, and 1.5 or 4.5% GFP^{pos} cells for IDLV after one or two hits, respectively. Colonies derived from the low proportion of FACS-sorted GFP^{pos} B2M-edited HSPCs mostly contained full-length vector trapping events (positive for the payload and viral features ddPCR probes) and confirmed the tendency of AAV to integrate more frequently as longer concatemers (FIG. 7m and FIG. 9e). Even more strikingly, we found only 1 integration event out of 83 colonies (1.2%) for IDLV and 8 out of 44 (18.2%) for AAV among the GFP^{neg} colonies, further confirming the higher tendency of AAV to give rise to unwanted integration of DNA fragments, particularly ITRs.

To obtain an even more comprehensive assessment of the full spectrum of genetic outcomes of editing at the targeted locus, we also probed colonies edited at AAVS1 for bearing long-range deletions encompassing the targeted locus, which would escape determination with the previously described targeted analyses. Strikingly, when probing for a sequence about 800 bp telomeric to the nuclease target site, we found that from 10 to 15% of AAVS1-edited colonies harbored only one copy (FIG. 7n). Moreover, probing for AURKC, a gene 2 Mbp telomeric to the target site, showed that one of these colonies lacked one copy of this sequence, suggesting sporadic occurrence of megabase-scale deletions or rearrangement in colony-forming edited HSPCs (FIG. 9f).

We then edited human mPB HSPCs pooled from 3 donors with the different enhanced protocols and HDR templates and transplanted them into NSG mice to evaluate repopulation capacity. While the best performing IDLV and AAV optimized protocols, both including Ad5-E4orf6/7 proteins, showed a similarly rate of GFP⁺ cells with medians of 18% in the graft early post-transplant (FIG. 70, p), there was a progressive decrease with time in the AAV group, consistent with previous findings in this and other papers (Ferrari et al., 2020, Nature Biotechnology). On the contrary, IDLV treatment showed stable marking throughout the follow-up with a median of 15% at the end of the study (FIG. 70, p and FIG. 9g, h), thus outperforming AAV at editing LT-HSCs. This outcome was confirmed throughout hematopoietic lineages (FIG. 9i, j), with no differences in composition across treatments (FIG. 9k, I). We then measured the copies of IDLV and AAV within the human graft of the respective groups and found detectable signal for both platforms in a similar range (0.1-0.2 CG) in these optimized conditions yielding highly edited grafts (FIG. 7q and FIG. 9m). However, deep sequencing of the edited locus from mice splenocytes showed higher number and proportion of alleles harboring integrated DNA fragments for the AAV-based protocol than the IDLV ones (FIG. 9n, o), which became even more evident when computing the fraction of alleles carrying ITR or LTR sequences (FIG. 7r). Moreover, the origin of trapped fragments was substantially different when aligning the integrated sequences to their reference AAV or IDLV genome and confirmed a higher propensity towards integration of AAV ITRs than IDLV LTRs (FIG. 7s). Aborted HDR events (as defined in FIG. 4b) were retrieved for both viral templates. Two alleles in the AAV group showed integration of fragments derived from reverse packaged AAV transfer plasmid backbone, encompassing a portion of the ITR and the neighboring F1 phage-derived ori sequence, or the kanamycin resistance gene from plasmids used for AAV production. In agreement with these data, GFP^{pos} colonies generated from CD34⁺ harvested from the bone marrow of human hematochimeric mice 14 weeks post-transplant confirmed engraftment and persistence of some clones carrying integrated AAV features (FIG. 7t and FIG. 9p).

Overall, while both DNA delivery templates showed some propensity for NHEJ-mediated trapping at the edited loci, IDLV induced substantially lower and less persistent vector DNA load, triggered a shorter DDR and yielded significantly less insertions of vector fragments in the genome as compared to AAV when both platforms reached similarly editing efficiencies in HSPCs. Because at variance with AAV ITR, IDLV LTR are transcriptionally silent, the concern for vector-dependent genotoxic outcome nearby the edited locus and genome-wide are significantly alleviated by the choice of the latter platform. Moreover, IDLV treatment had a lower impact on the clonogenic capacity of the treated cells and achieved significantly higher rates of editing in LT-HSCs.

### Materials and Methods (Examples 1 to 5)

### Crispr/Cas9 Nucleases

Genomic target sequences of sgRNAs were previously reported (AAVS1-HS, IL2RG, CD40LG, B2M) or will be reported in detail elsewhere (RAG1) (Schiroli, et al., Cell Stem Cell 24, 551-565.e8, 2019; Vavassori et al., 2021, EMBO Molecular Medicine, 13: e13545; Gaudelli et al., 2020, Nature Biotechnology, 38 (7): 892-900). The genomic target sequence of AAVS1-LS sgRNA is the following: 5′-GTCCCCTCCACCCCACAGTG GGG-3′. RNP complexes to be delivered by electroporation were assembled by incubating at V1: 1.5 (AAVS1, IL2RG, B2M, RAG1) or 1:2 (CD40LG) molar ratio Streptococcus pyogenes (Sp) Cas9 protein (Aldevron) with pre-annealed synthetic Alt-R crRNA: tracrRNA (Integrated DNA Technologies) for 10 min at 25° C. For some AAVS1 experiments, synthetic single gRNA from Synthego was used and assembled with Cas9 protein following the same procedure.

### Hdr Viral Donor Templates

The design of ssAAV2/6 transfer vector constructs for AAVS1, CD40L and IL2RG editing were previously reported and are described in detail in FIG. 10a (Schiroli, et al., Cell Stem Cell 24, 551-565.e8, 2019; Ferrari et al., 2020, Nature Biotechnology; Vavassori et al., 2021, EMBO Molecular Medicine, 13: e13545; Gaudelli et al., 2020, Nature Biotechnology, 38 (7): 892-900). For some AAVS1 experiments the barcoded ssAAV2/6 cassette described in Ferrari et al., 2020 was used interchangeably with the non-barcoded one. The design of ssAAV2/6 construct for RAG1 editing will be described elsewhere. All the cargo cassettes are flanked by AAV serotype-2 derived ITRs. The 5′ITR sequence bears a 11-bp deletion (5′-AAAGCCCGGGC-3′) that can be rescued during viral genome replication by exploiting the wild-type 3′ITR sequence.

The ssAAV5/6 transfer vector construct for AAVS1 editing was designed to encompass the same cargo cassette of ssAAV2/6. 5′ and 3′ ITR originating from the wild-type genome of AAV5 (GenBank ID NC_006152.1, 167 nucleotides on both sides) were cloned in place of ITR2 (FIG. 10b).

The scAAV2/6 transfer vector construct for AAVS1 editing was designed to encompass the enhanced GFP transgene under the control of the human PGK promoter and a bovine growth hormone (BGH) polyadenylation signal (FIG. 10c). Homology arms for AAVS1 locus flank the reporter cassette. The left homology arm was shortened to fit within the scAAV size limit of encapsidation (about 2.8 kb). The 5′-ITR sequence contains the trs deletion (5′-CCAACTCCATCACTAGG-3′) to avoid strand cleavage during viral genome replication, thus enabling AAV to package as DNA dimers.

The IDLV transfer vector construct for AAVS1 editing was previously reported in Ferrari et al., 2020 and is described in detail in FIG. 7a.

Vector maps were designed with SnapGene software v.6.0.2 (from GSL Biotech, available at snapgene.com).

Cell lines and primary cell culture

HEK293T and K-562 cells were cultured in Iscove's modified Dulbecco's medium (IMDM, Corning) supplemented with 10% heat-inactivated fetal bovine serum (FBS) (Euroclone), 100 IU ml⁻¹ penicillin, 100 ug ml⁻¹ streptomycin and 2% glutamine. For AAV production, HEK293 cells were cultured in DMEM supplemented with 2% heat inactivated FBS, 1% penicillin and streptomycin. For IDLV production, HEK293T cells were cultured in IMDM without phenol red supplemented with 10% FBS, 100 IU ml⁻¹ penicillin and 100 ug ml⁻¹ streptomycin.

CB CD34⁺ HSPCs were purchased frozen from Lonza according to the TIGET-HPCT protocol approved by OSR Ethical Committee and were seeded at the concentration of 5×10⁵ cells per ml in serum-free StemSpan medium (StemCell Technologies) supplemented with 100 IU ml⁻¹ penicillin, 100 μg ml⁻¹ streptomycin, 2% glutamine, 100 ng ml⁻¹ hSCF (PeproTech), 100 ng ml⁻¹ hFlt3-L (PeproTech), 20 ng ml⁻¹ hTPO (PeproTech) and 20 ng ml⁻¹ hIL-6 (PeproTech), 10 μM PGE2 (at the beginning of the culture, Cayman), 1 μM SR1 (Biovision) and 50 nM UM171 (STEMCell Technologies).

G-CSF or G-CSF+Plerixafor mPB CD34⁺ HSPCs were purified in house with the CliniMACS CD34 Reagent System (Miltenyi Biotec) from Mobilized Leukopak (AllCells) according to the TIGET-HPCT protocol approved by OSR Ethical Committee and following the manufacturer's instructions. HSPCs were seeded at the concentration of 5×10⁵ cells per ml in serum-free StemSpan medium (StemCell Technologies) supplemented with 100 IU ml⁻¹ penicillin, 100 μg ml⁻¹ streptomycin, 2% glutamine, 300 ng ml⁻¹ hSCF, 300 ng ml⁻¹ hFlt3-L, 100 ng ml⁻¹ hTPO and 10 μM PGE2 (at the beginning of the culture), 1 μM SR1 and 35 nM UM171.

All cells were cultured in a 5% CO₂ humidified atmosphere at 37° C.

### AAV Production and Quality Controls

Most recombinant AAV2/6 and the ssAAV5/6 were produced at the Vector Core of the UMR1089 (CPV, INSERM, University of Nantes). For ssAAV2/6, the pDP6 helper plasmid was used. For ssAAV5/6, a new construct (named Rep5/Cap6-Ad) was generated to encompass: the full wild-type AAV5 rep (GenBank ID NC_006152.1, cloned in place of the AAV2 rep in the pDP6), the AAV6 cap gene and the adenoviral helper functions (i.e., E2A, VA RNA, and E4 Ad). This new Rep5/Cap6-Ad helper plasmid allowed the expression of VP1, VP2 and VP3 at the expected Jan. 1, 2010 ratio (FIG. 2p).

HEK293 cells were seeded in Cell-Stacks 5 and transfected with the two plasmids by calcium phosphate precipitation method. Cell pellets were harvested after 3 d and a freeze/thaw action was performed to lyse the cells and release AAV6 particles, which were precipitated by polyethylene glycol and then purified by double CsCl density gradient ultracentrifugation. Instead, for experiments in FIG. 2e-i, purification was performed by affinity chromatography (AVB). AAV6 were formulated in 1X DPBS (Thermo Scientific, Illkirch, France), sterile filtered (0.22 μm), aliquoted and frozen at −80° C. Vector genome titers (vg ml⁻¹) were determined using a qPCR assay specific for ITR or GFP.

Empty (FIG. 10d, top) and full (FIG. 10d, middle) ssAAV2/6 particles fractionated by CsCl gradient, and ssAAV2/6 purified by AVB affinity chromatography (FIG. 10d, bottom) have been analyzed by analytical ultracentrifugation (AUC).

For SDS-PAGE analysis of AAV preparations, vectors were denatured for 5 min at 95° C. in Laemmli buffer and loaded on 10% Tris-Glycine polyacrylamide gels (Life Technologies). Following electrophoresis, gels were transferred on nitrocellulose membranes for western blot analysis. Membranes were probed with monoclonal antibody B1 (Kleinschmidt), which recognizes VP1, VP2 and VP3 capsid proteins. Goat anti-mouse-HRP secondary antibody was used (Dako, P0447).

Infectious Center Assays (ICA) were performed for AAV2/6 preparations, as previously described (Zolotukhin et al., 1999, Gene Therapy, 6 (6): 973-985). Briefly, HeLa RC32 cells were seeded in 48-well plates at 6×10⁴ cells/well. and infected the day after in duplicate 10-fold dilutions of the AAV preparations, in presence of wild-type Ad5 at a multiplicity of infection (MOI) of 500 transducing units per cell. Cells were harvested 24-26 hours post-infection and filtered through Zeta-Probe nylon membranes (Bio-Rad) using a vacuum device. Membranes were hybridized overnight with vector-specific probes generated with the PCR Fluorescein Labeling Mix (Sigma-Aldrich), and detection was performed using the CDP-Star labeling kit (Sigma-Aldrich). Titers were determined by counting dots (i.e., AAV-infected cells) on membrane autoradiography.

### Idlv Production and Quality Controls

IDLVs were manufactured by transient quadri-transfection of HEK293T cells, followed by DNase treatment, anion exchange chromatography, concentration, gel filtration and final sterilizing filtration; the purification workflow was previously optimized in order to remove >99% of DNA and protein impurities while preserving vector biological activity (Soldi et al., 2020, Molecular Therapy, 19:411-425). HEK293T cells were seeded in 6 ten-tray cell factories (Corning) and transiently transfected in the presence of Calcium Phosphate with the following plasmids: the transfer vector construct described above and in Ferrari et al., 2020 the envelope plasmid coding for VSV.G, the third-generation packaging plasmids pRSV.REV and pGag-Pol pMDLg/pRRE.D64VInt coding for a catalytically inactive integrase. In addition, the pAdvantage plasmid was used (Nature Technologies). After 14 h, the transfection mixture was removed and replaced by fresh medium supplemented with 1 mM Sodium Butyrate (Sigma). After 30 h, 6 I of culture supernatant were harvested, clarified through 0.8-0.45 μm filtration (Sartorius) and treated with Benzonase (Merck) at 16 U ml⁻¹ final concentration for 4 h at 4° C. Anion exchange chromatography was performed using the AKTA Avant 150 system (Cytiva): lentiviral particles were loaded on a column containing Toyopearl DEAE-650C resin (Tosoh) and eluted by DPBS/NaCl linear gradient. The eluted vector was diluted in PBS, further treated with Benzonase at 50 U mL⁻¹ for 2 h at 4° C. and concentrated through a MWCO 100 kDa VivaFlow cassette (Sartorius). Gel filtration was performed using the AKTA Avant 150 system and a column filled with Sepharose 6FF resin (Cytiva); the IDLV was eluted in DPBS, sterilized by filtration using 0.2 μm polyethersulphone filter (Sartorius), concentrated by MWCO 100 kDa Vivaspin (Sartorius) obtaining a final volume of 2.3 ml, aliquoted and stored at −80° C.

The infectious titer was determined as previously described in Soldi et al. with minor modifications. HEK293T cells were transduced with serial dilutions of the purified IDLV in the presence of polybrene; after 3 d, the cells were collected, the DNA extracted and the vector copy number (VCN) determined by ddPCR, using primers described previously (Mátrai et al., 2011, Hepatology (Baltimore, Md.) 53:1696-1707) and human TELO as normalizer. The infectious titer was expressed as TU ml⁻¹ and calculated as: VCN x number of cells x (1/dilution factor). As positive control a CEM cell line stably carrying four vector integrants was used. The physical titer was measured by HIV-1 Gag 24 antigen immunocapture assay (Perkin Elmer) following manufacturer's instructions. IDLV specific infectivity was calculated as ratio between the infectious titer and physical titer. The total particles concentration and aggregation were measured by multi-angle dynamic light scattering (MADLS) technology using Zetasizer Ultra (Malvern Panalytical) following manufacturer's instructions. The endotoxin level was determined by LAL kinetic chromogenic method, using the Endosafe® PTS™ system and a single use cartridge with sensitivity of 0.005-0.5 EU ml⁻¹ (Charles River).

The results of analytical tests conducted on the IDLV preparation used throughout the present study are shown in Table 4.

TABLE 4
Quality controls for IDLV preparations.

| Analytical test | Result |
|---|---|
| Infectious Titer (TU/mL) | 3.1E+09 |
| Physical Titer (mg p24/mL) | 47.0 |
| Infectivity (TU/ng p24) | 6.7E+04 |
| Total Particles Concentration (pp/mL) | 4.75E+11 |
| Aggregates (%) | 1.3 |
| Endotoxin (EU/1E8 TU) | 11.7 |

### MRNA In Vitro Transcription

All constructs for mRNA in vitro transcription and the methods for their preparation, quantification and quality assessment were previously described (Schiroli et al., 2019 and Ferrari et al., 2020).

### Gene Editing of Human HSPCs and Analyses

Gene editing protocols for human HSPC has been previously described in detail and is shown in FIG. 10e (Ferrari et al., 2021, Nature Protocols 16 (6): 2991-3025). Briefly, for AAV6-based gene editing, after 3 d of stimulation 1×10⁵-5×10⁵ cells were washed with ten volumes of DPBS and electroporated using P3 Primary Cell 4D-Nucleofector X Kit and Nucleofector 4D device (program EO-100) (Lonza). Cells were electroporated according to the manufacturer's instructions with RNPs at a final concentration of 1.25-2.5 μM together with 0.1 nmol of Alt-R Cas9 Electroporation Enhancer (Integrated DNA Technologies) only for two-parts sgRNAs. AAV6 transduction was performed at a dose of 2×10⁴ vg per cell 15 min after electroporation, unless otherwise specified.

For one-hit IDLV-based gene editing, after 2 or 2.5 d of stimulation 1×10⁵-5×10⁵ cells were treated with 8 μM cyclosporin H (CsH, Sigma) and then transduced with purified IDLV at MOI of 150, unless otherwise specified. After 24 or 12 h, cells were washed with DPBS and electroporated using P3 Primary Cell 4D-Nucleofector X Kit and program EO-100 (Lonza), as described above. For two-hits IDLV-based gene editing, another round of transduction in presence of 8 μM CsH was performed immediately after electroporation with purified IDLV at MOI of 150, unless otherwise specified.

When indicated, in vitro transcribed mRNAs were added to the electroporation mixture at the following final concentrations: 150 μg/μl GSE56; 250 μg/μl GSE56/Ad5-E4orf6/7; 100 μg/μl Ad5-E4orf6; 150 μg/μl Ad5-E1B55K (see Table 5 for further details). Four days after the editing procedure, cells were collected to analyze by flow cytometry the percentage of cells expressing the GFP marker within HSPC subpopulations and to extract genomic (g) DNA for molecular analyses, unless otherwise indicated.

TABLE 5
List of mRNA constructs and related quantity/concentrations.

| mRNA | mRNA | Amount | Conc. | mRNA | Moles |
|---|---|---|---|---|---|
| construct | length | [mg] | [mg/ml] | copies | (pmol) |
| GSE56 | 1075 | 3 | 150 | 5.1E+12 | 8.5 |
| Ad5-E4orf6/7 | 1148 | 1.5 | 75 | 2.4E+12 | 4.0 |
| GSE56/Ad5- | 1529 | 5 | 250 | 6.0E+12 | 9.9 |
| E4orf6/7 | | | | | |
| Ad5-E1B55K | 2241 | 3 | 150 | 2.4E+12 | 4.1 |
| Ad5-E4orf6 | 1620 | 2 | 100 | 2.3E+12 | 3.7 |

### Flow Cytometry

Immunophenotypic analyses were performed on the fluorescence activated cell sorting (FACS) Canto II (BD Pharmingen). From 0.5×10⁵ to 2×10⁵ cells (either from culture or mouse samples) were analyzed by flow cytometry. Cells were stained for 15 min at 4° C. with antibodies in a final volume of 100 μl and then washed with DPBS+2% heat inactivated FBS. Single stained and fluorescence-minus-one-stained cells were used as controls. The Live/Dead Fixable Dead Cell Stain Kit (Thermo Fisher) or 7-aminoactinomycin D (Sigma Aldrich or Biolegend) were included during sample preparation according to the manufacturer's instructions to identify dead cells.

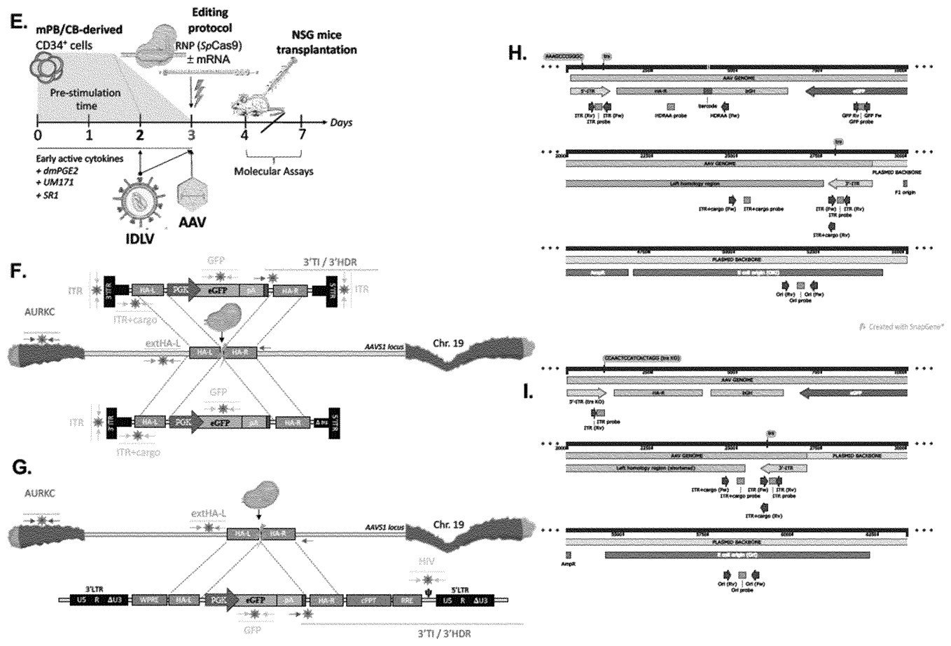

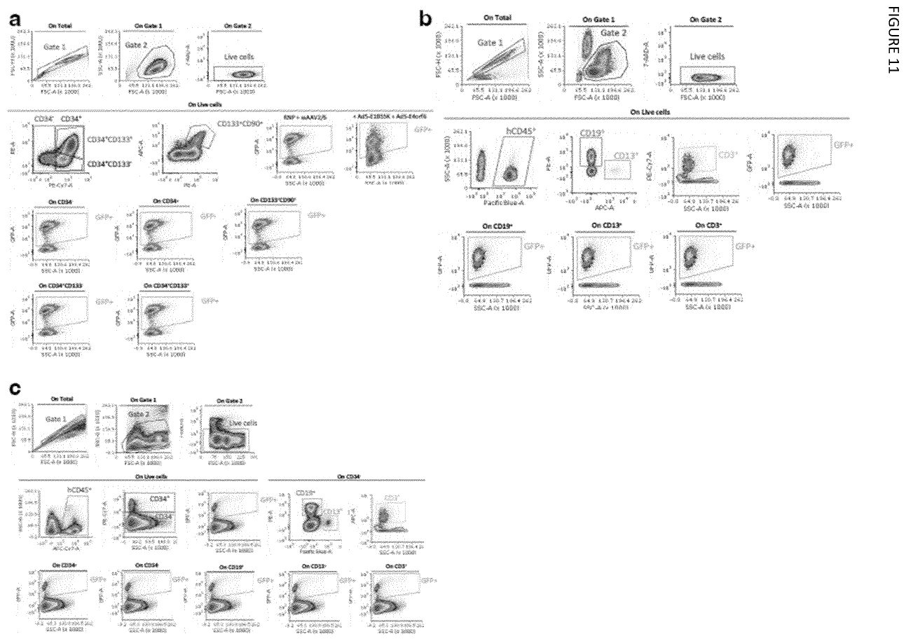

Cell sorting was performed on a BD FACSAria Fusion (BD Biosciences) using BDFACS Diva software and equipped with four lasers: blue (488 nm), yellow/green (561 nm), red (640 nm) and violet (405 nm). Cells were sorted with an 85 mm nozzle. Sheath fluid pressure was set at 45 psi. A highly pure sorting modality (four-way purity sorting) was chosen. Sorted cells were collected in 1.5 ml Eppendorf tubes containing 500 μl of DPBS or HSPC medium. Gating strategies for flow cytometry analyses are provided in FIG. 11. Data were analyzed with FCS Express 6 Flow or 7 Flow.

### Quantification of NHEJ and HDR Editing Efficiency

gDNA was isolated with QIAamp DNA Micro Kit (QIAGEN) according to the manufacturer's instructions. Unless otherwise specified, nuclease activity was measured using a mismatch-sensitive endonuclease T7 assay (New England Biolabs) on PCR-based amplification products of the targeted locus, as described in Ferrari et al., 2021. Digested DNA fragments were resolved and quantified by capillary electrophoresis on 4200 TapeStation System (Agilent) according to the manufacturer's instructions.

For HDR ddPCR analysis, 5-50 ng of gDNA were analyzed using the QX200 Droplet Digital PCR System (Bio-Rad) according to the manufacturer's instructions. HDR ddPCR primers and probes were designed on the junction between the vector sequence and the targeted locus, as shown in FIG. 10f, g and described in Vavassori et al., 2021 and Ferrari et al., 2020 and 2021. Human TTC5 (Bio-Rad) was used for normalization.

### Gene Expression Analyses

Total RNA was extracted using RNeasy Plus Micro Kit (QIAGEN), according to the manufacturer's instructions and DNAse treatment was performed using RNase-free DNAse Set (QIAGEN). Complementary DNA was synthesized with SuperScript VILO IV cDNA Synthesis Kit (Thermo Fisher) with EzDNAse treatment. cDNA was then used for quantitative PCR (qPCR) in a Viia7 Real-time PCR thermal cycler using TaqMan Gene Expression Assays (Applied Biosystems) mapping to genes. Data were analyzed with QuantStudio Real-Time PCR software v.1.1 (Applied Biosystem). Relative expression of each target gene was first normalized to HPRT1 and then represented as fold changes of the ΔΔCt relative to the untreated cells.

### Clonogenic Assay

Colony-forming-unit cell assay was performed 1 d after editing procedure by plating 600 HSPCs in methylcellulose-based medium (MethoCult H4434, StemCell Technologies) supplemented with 100 IU ml⁻¹ penicillin and 100 μg ml⁻¹ streptomycin. For the experiment in FIG. 7t, CD34⁺ GFP⁺ cells were FACS-sorted from BM cells of xenotransplanted mice, and 600 cells were plated as described above. Three technical replicates were performed for each condition. Two weeks after plating, colonies were counted and classified according to morphological criteria. For experiments in FIG. 4l-m and FIG. 9d, single colonies were then manually picked.

### Immunofluorescence Analysis

Multitest slides (MP Biomedicals, 096041505) were coated with Poly-L-lysine solution (Sigma-Aldrich, P8920-500ML). After three washes with PBS solution, 0.3-0.5×10⁵ cells were seeded on covers for 20 minutes and fixed with 4% PFA (Santa Cruz Biotechnology, sc-281692) for 20 minutes. Cells were then permeabilized with 0.1% Triton X-100. After blocking with 0.5% BSA and 0.2% fish gelatin in PBS, cells were stained with the indicated primary antibodies (53BP1 Antibody, Bethyl Laboratories; Anti-phospho Histone H2A.X (Ser139) Antibody, clone JBW301, Merck; NBS1 Antibody, Novus Biologicals). Cells were than washed with PBS and incubated with Alexa Fluor 568- and 647-labeled secondary antibodies (Invitrogen/Thermo Scientific). Nuclear DNA was stained with DAPI at 0.2 μg/ml concentration (Sigma-Aldrich, D9542) and covers were mounted with Aqua-Poly/Mount solution (TebuBio, 18606-20) on glass slides (Bio-Optica). Fluorescent images were acquired using Leica SP5 Confocal microscopes. Quantification of DDR foci in immunofluorescence images was conducted using Cell Profiler.

### Mice

All experiments and procedures involving animals were performed with the approval of the Animal Care and Use Committee of the San Raffaele Hospital (IACUC no. 749 and 1206) and authorized by the Italian Ministry of Health and local authorities accordingly to Italian law. NOD-SCID-IL2Rg−/− (NSG) female mice (The Jackson Laboratory) were held in specific pathogen-free conditions.

### Cd34⁺ HSPC Xenotransplantation Experiments in NSG Mice

For transplantation of CB and mPB CD34⁺ HSPCs, the outgrowths of 1.5×10⁵ and 1×10⁶ culture-initiating HSPCs, respectively, were injected intravenously 24 h after editing into sub-lethally irradiated NSG mice (150-180 cGy). The lower, limiting doses were used for CB-derived cells when aiming to better report difference between protocols. Sample size for each experiment was determined by the total number of available treated cells. Mice were randomly distributed to each experimental group. Human CD45⁺ cell engraftment and the presence of edited cells were monitored by serial collection of blood from the mouse tail or the retro-orbital plexus and, at the end of the experiment (>18 weeks after transplantation for CB HSPCs and >14 weeks for mPB HSPCs after transplantation), bone marrow and spleen were collected for end-point analyses.

### Quantification of Viral Vectors Copies Per Human Genome

gDNA was isolated with QIAamp DNA Micro Kit from DNAse-treated in vitro culture cells pellet and in vivo samples, or with QuickExtract (Epicentre) from single colonies according to the manufacturer's instructions. For ddPCR analyses, 5-50 ng of gDNA for in vitro/vivo samples and 2 μl of gDNA for single colonies were analyzed using the QX200 Droplet Digital PCR System according to the manufacturer's instructions. Primers and probes were designed as shown in FIG. 10f-h. Human TTC5 (Bio-Rad) was used for normalization, except for experiments in FIGS. 10 and 4k in which GAPDH (Bio-Rad) was used.

### Retrieval of AAV and IDLV Integration Sites: Library Preparation and Bioinformatic Analyses By RAAVloli Pipeline

To assess AAV integration, we adopted a Sonication-based Linker-Mediated PCR method (SLIM), as previously described (Cesana et al., 2021, Nature medicine, 27:1458-1470). Briefly, genomic DNA was sheared using a Covaris E220 Ultrasonicator (Covaris Inc.), generating fragments with a target size of 1,000 bp. The fragmented DNA was subjected to end repair, 3′ adenylation and ligation (NEBNext® Ultra™ DNA Library Prep Kit for Illumina®, New England Biolabs) to custom linker cassettes (LC) (Integrated DNA Technologies). LC sequences contained a 8-nucleotide barcode for sample identification. Ligation products were subjected to 35 cycles of exponential PCR with primers (available upon request) complementary to different regions of the AAV or IDLV genomes (FIGS. 5a and 7c) and to the LC. For each set of AAV or IDLV specific primers, the procedure was performed in technical replicates (n=2-3) using 80-100 ng of sheared DNA/each. Next, ten additional PCR cycles were done to include sequences required for sequencing and a second 8-nucleotide DNA barcode. PCR products were quantified by qPCR using the Kapa Biosystems Library Quantification Kit for Illumina, following the manufacturer's instructions. qPCR was performed in triplicate on each PCR product diluted 10:3, and the concentrations were calculated by plotting the average Ct values against the provided standard curve. Finally, the amplification products were sequenced by Illumina Next/Novaseq platforms (Illumina).

After sequencing, a dedicated bioinformatics pipeline was developed to analyze the amplified sequences for integration sites identification. Specific details of the pipelines are going to be reported in a follow-up methodological paper. The main steps were: 1) quality checks of input sequences were run using FastQC (https://www.bioinformatics.babraham.ac.uk/projects/fastqc/) while adapters and PhiX reads were removed with Flexbar, and the initial 12 random nucleotides with Trimmomatic; 2) sequences were then aligned to the AAV genome to identify the AAV or IDLV portions using BWA-MEM (Burrows-Wheeler aligner-maximal exact match); 3) among AAV and IDLV containing sequences, we selected only those starting with the sequence of the last nucleotides adopted for PCR amplification plus 10 vector-specific nucleotides; we then aligned the selected reads on a hybrid genome composed of both the AAV and human genome (release hg19/GRCh37 downloaded from UCSC (University of California, Santa Cruz) Genome Browser website). The alignments were then processed with a custom Python software to identify integration loci and vector rearrangements using the CIGAR (Concise Idiosyncratic Gapped Alignment Report) string (FIG. 6a). Unique IS were identified considering the AAV/human genome breakpoint and the number and type of AAV rearrangements, such that two reads of the same PCR were assigned to the same IS if both alignments on human genome start in the same genomic loci (+/−4) and in both reads the AAV junction is aligned within a window of 8 bases. Moreover, potential indels in between AAV junction and genomic locus were included in the identification window to distinguish one or two IS. To remove potential PCR artefacts, all analyses were performed considering integration events identified by at least 3 independent fragments of DNA. We applied this approach to source-aligned reads. The abundance of each IS was performed by counting for the same vector/cell genome junctions (IS) the number of different DNA fragments containing a genomic segment variable in size depending on the shear site position and that will be unique for each different cell genome present in the starting cell population. Therefore, the number of different shear sites assigned to an IS will be proportional to the initial number of contributing cells in the population studied, thus estimate the clonal abundance of each IS in the starting sample avoiding the biases introduced by PCR amplification.

### Deep Sequencing of the Target Locus and Bioinformatic Analyses

PCR amplicons for individual samples were generated by nested PCR and starting from >50-100 ng of purified gDNA. The first PCR step was performed with GoTaq G2 DNA Polymerase (Promega) according to manufacturer instruction using the following amplification protocol: 95° C.×5′min, (95° C.×0.5 min, 60° C.×0.5 min, 72° C.×0.2 5 min)×20 cycles, 72° C.×5 min. The second PCR step was performed with GoTaq G2 DNA Polymerase (Promega) according to manufacturer instruction using 5 μl of the first-step PCR product and the following amplification protocol: 95° C.×5 min, (95° C.×0.5 min, 60° C.×0.5 min, 72° C.×0.3 min)×20 cycles, 72° C.×5 min. Second-step PCR primers were endowed with tails containing P5/P7 sequences, i5/i7 Illumina tags to allow multiplexed sequencing and R1/R2 primer binding sites. PCR amplicons were separately purified performing double-side selection with AmpPure XP beads (Beckman Coulter) or QIAquick PCR Purification Kit (QIAGEN). Amplicons concentration and quality was assessed by QuantiFluor ONE dsDNA system and 4200 Tapestation System (Agilent). Amplicons from up to 36 differently tagged samples were multiplexed at equimolar ratios and run by the Center for Omic Sciences (COSR) at San Raffaele or by Genewiz (Azenta Life Sciences) on MiSeq 2×300 bp paired end sequencing (Illumina).

Sequencing data were analyzed with CRISPResso2, which enables detection and quantification of insertions, mutations, and deletions in reads from gene editing experiments. In details, for each sample, input NGS reads were trimmed using the Trimmomatic software (http://www.usadellab.org/cms/?page=trimmomatic) based on the phred33 score to get rid of low-quality positions (score <30) and to remove Illumina adapters, keeping only trimmed sequences longer than 100 bp (CRISPResso2 options:—trim_sequences—trimmomatic_command trimmomatic—trimmomatic_options_string ‘ILLUMINACLIP: TruSeq3-PE-2.fa: 2:30:10 MINLEN: 100’). Then, each couple of paired-end reads was merged using the FLASh software to produce a single contig, which was mapped to the input amplicon reference (AAV or IDLV, depending on the experiment). The sgRNA sequence was provided to focus the analysis on the target region, and the quantification window was set to 1 bp per side around the cut site. Identified alleles were quantified by measuring the number of reads and their relative abundance based on total read counts. Finally, we post-processed the CRISPResso2 allele outputs by correcting all the mismatch positions outside the quantification window, which are likely to be the result of amplification/sequencing errors, and we re-quantified the total read counts and the corresponding relative abundances. Alleles showing a relative abundance lower than the false positive threshold (set at 0.2% based on UT samples) were filtered out.

For quantification and characterization of trapped fragments into CRISPResso2 alleles, we extracted insertions longer than 20 bp and with relative abundance >0.2% and we locally aligned these fragments against the viral vector genome (AAV or IDLV, depending on the experiment) with the Bowtie2 software (options:--local-L 10). Coverage of vector genome was assessed with the genomeCoverageBed utility from the bedtools suite, and the corresponding normalized abundance (LogCPM) was computed. Since deep sequencing of the edited locus drops out the alleles carrying targeted integration of the cassette, we calculate the percentage of alleles with trapping events in the total human graft of each mouse using the following formula: % alleles with trapping events measured by targeted deep sequencing x 100/(100-% HDR (measured by ddPCR)).

### GUIDE-Seq

For GUIDE-Seq analysis 3×10⁵ K562 cells were electroporated with 25 pmol CRISPR-Cas9 delivered as RNP and 200 pmol dsODN using SF Cell Line 4D-Nucleofector X Kit and Nucleofector 4D device (program FF-120), according to the manufacturer's instructions. Successful dsODN integration at the on-target sites was confirmed by restriction fragment length polymorphism assay using Ndel enzyme (NEB). Library preparation was performed as in Tsai et al., 2015, Nature Biotechnology, 33:187-198). GUIDE-Seq computational analysis was performed as previously described in Schiroli et al., 2019.

### Quantifications and Statistical Analyses

The number of biologically independent samples, animals or experiments is indicated by “n”. For some experiments, different HSPC donors were pooled to account for donor-related variability and reach the number of cells needed for the analyses. Data were summarized as median with 95% CI or mean±s.e.m. depending on data distribution. Inferential techniques were applied in presence of adequate sample sizes (n ≥5), otherwise only descriptive statistics are reported. Two-tail tests were performed throughout the study. The Mann-Whitney test was performed to compare two independent groups, while in presence of more than two independent groups the Kruskal-Wallis test followed by post hoc analysis using Dunn's test was used. In presence of dependent observations, the Friedman test with Dunn's multiple comparisons or linear mixed-effects models (LME) were performed. The last procedures were applied to properly account for the dependence structure among observations, by including additional (nested) random-effect terms, thus considering in the model unobservable sources of heterogeneity among experimental units. When analyzing time courses, treatment group indicator and time variables, along with their interaction, were included as covariates in the model to identify potential differences in growth dynamics of treatment groups. A random intercept model was estimated and, when necessary, nested random effects were considered (for example, to account for repeated measures of cells per mouse within experiments). Quadratic polynomial models were also specified to better capture nonlinear trends. Standard transformations (logarithm, square/cubic root, ordered quantile normalization) were applied to outcome variables before entering in the LME model in order to satisfy model regression assumptions

Post hoc analysis after LME was performed, considering all the pairwise comparisons of treatment groups at a fixed timepoint. P values were adjusted using Bonferroni's correction. In all the analyses, the significance threshold was set at 0.05, while “NS” means not significant. Analyses were performed using GraphPad Prism v.9.3.1 (GraphPad) and R statistical software.

### Example 6

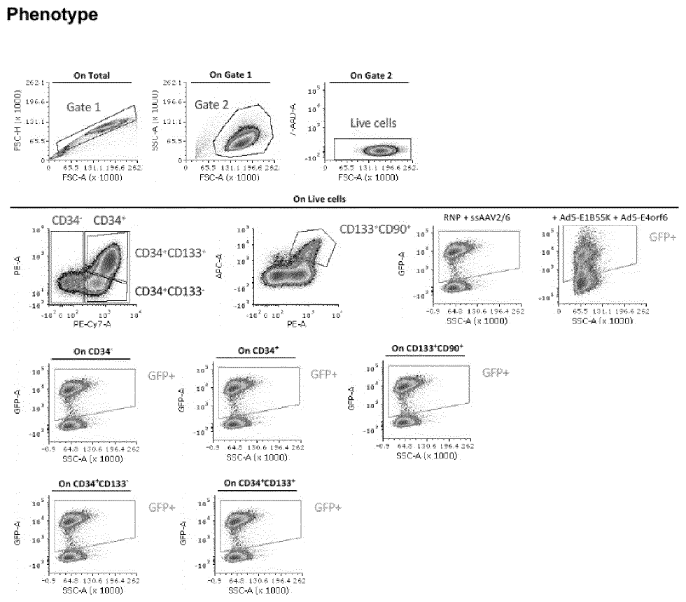

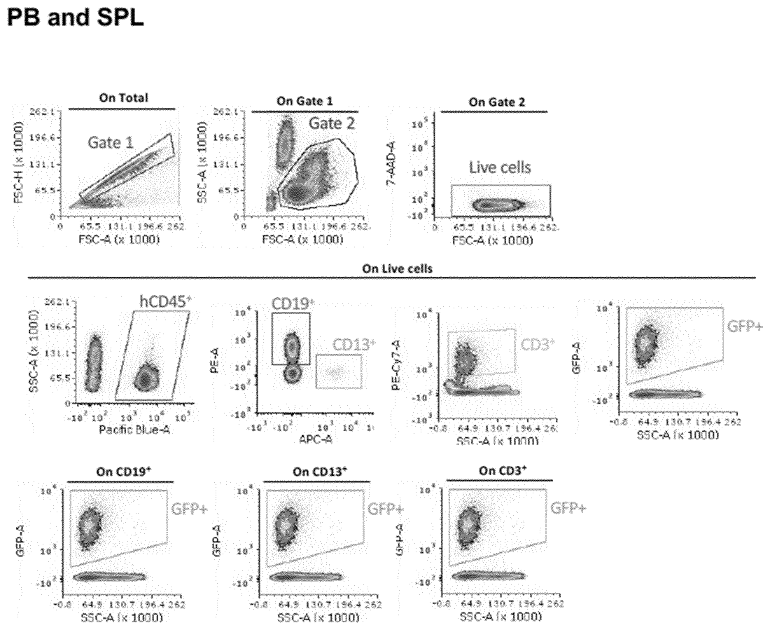

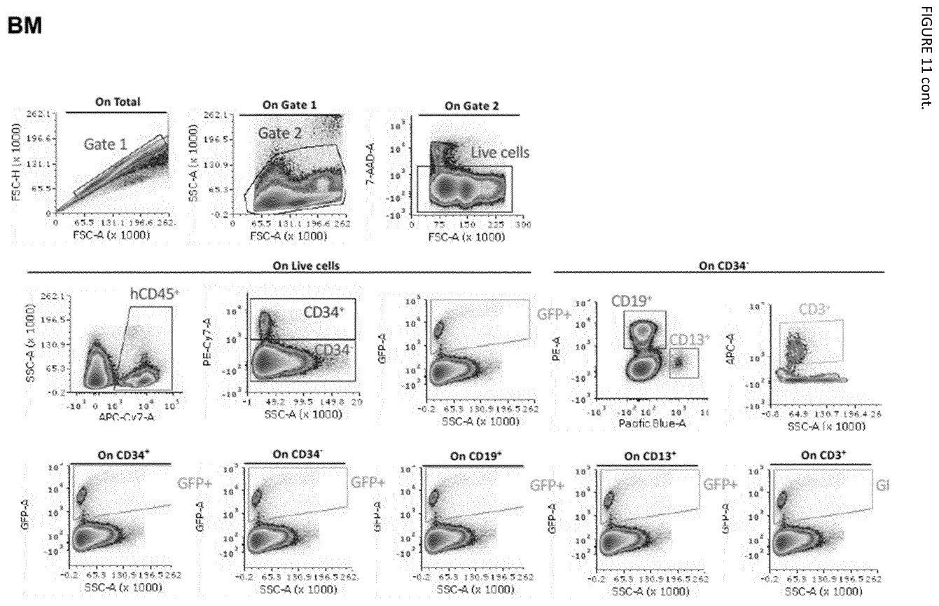

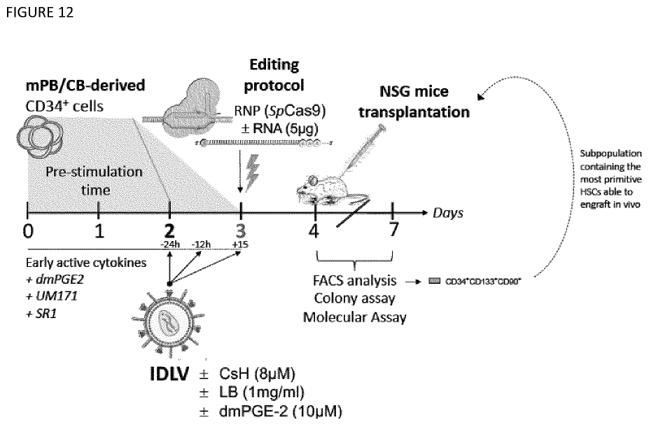

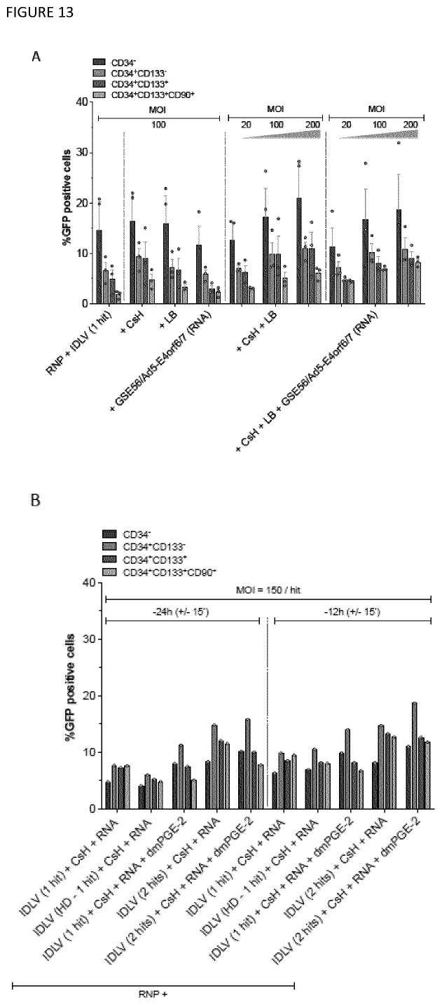

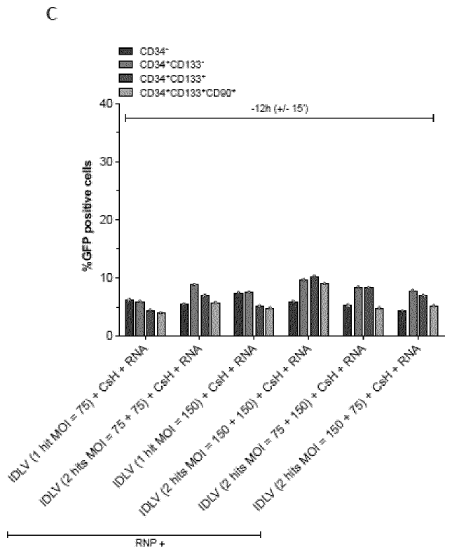

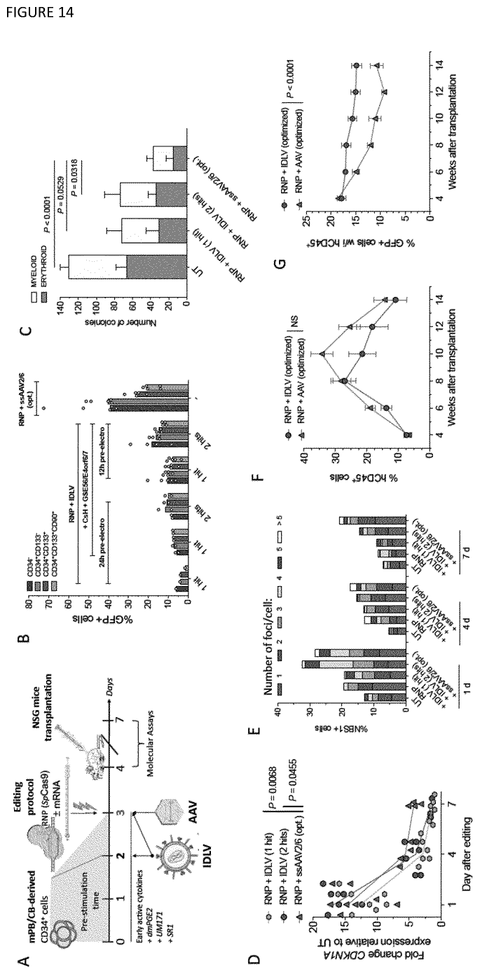

We performed AAVS1 editing in mPB HSPCs. We tested different combination of transduction (CsH, lentiboost, dmPGE-2) and editing (GSE56/E4orf6/7) enhancers in association with different MOIs of purified IDLV vector. Cytometry analysis revealed that combination of all enhancers improves GFP expressing cells into the most primitive HSC compartment (CD34⁺ CD133⁺ CD90⁺) which is the most relevant subpopulation of cells that can engraft in vivo. However, lentiboost has a minor impact when combined with other enhancers. Since the best performing MOI is spanning in the range of 100-200. While the timing of IDLV delivery during editing protocol (12 or 24 hours before RNP electroporation) were similar in terms of editing efficiency, the use of 1 or 2 hit(s) of IDLV template is striking to increase the percentage of GFP expressing cells, especially in primitive HSCs. The 2 hits protocol worked as best when using the highest MOI for each IDLV hit transduction. See FIGS. 12 to 14.

### Materials and Methods (Example 6)

### Primary Cell Culture

G-CSF or G-CSF+Plerixafor mPB CD34⁺ HSPCs were purified in house with the CliniMACS CD34 Reagent System (Miltenyi Biotec) from Mobilized Leukopak (AllCells) according to the TIGET-HPCT protocol approved by OSR Ethical Committee and following the manufacturer's instructions. HSPCs were seeded at the concentration of 5×10⁵ cells per ml in serum-free StemSpan medium (StemCell Technologies) supplemented with 100 IU ml⁻¹ penicillin, 100 μg ml⁻¹ streptomycin, 2% glutamine, 300 ng ml⁻¹ hSCF, 300 ng ml⁻¹ hFlt3-L, 100 ng ml⁻¹ hTPO and 10 μM PGE2 (at the beginning of the culture), 1 μM SR1 and 35 nM UM171. All cells were cultured in a 5% CO₂ humidified atmosphere at 37° C.

### Gene Editing of Human HSPCs and Analyses

For 1 hit IDLV-based gene editing, after 2 or 2.5 d of stimulation 1×10⁵-5×10⁵ cells were treated with transduction enhancer(s) (8 μM cyclosporin H and/or 1 mg/ml lentiboost and/or 10 M dmPGE-2) and then transduced with purified IDLV at MOI of 150, unless otherwise specified. After 24 or 12 h, cells were washed with DPBS and electroporated using P3 Primary Cell 4D-Nucleofector X Kit and program EO-100 (Lonza), with RNPs at a final concentration of 1.25-2.5 μM together. For 2 hits IDLV-based gene editing, another round of transduction in presence of transduction enhancer(s) was performed immediately (+15 min) after RNP electroporation with purified IDLV at MOI of 150, unless otherwise specified. When indicated, in vitro transcribed (m) RNA was added to the electroporation mixture at the following final concentration: 250 μg/μl GSE56/E4orf6/7. Four days after the editing procedure, cells were collected to analyze by flow cytometry the percentage of cells expressing the GFP marker within HSPC subpopulations and to extract genomic (g) DNA for molecular analyses, unless otherwise indicated.

### Example 7

### Materials and Methods

### Idlv Vector Production

IDLV donor was generated by the PDL Vector Core (SR-Tiget, Milan, Italy) using HIV-derived, third-generation self-inactivating transfer construct and the IDLV stock was prepared by transient transfection of HEK293T cells. At 30 hours post-transfection, vector-containing supernatant was collected, filtered, clarified, DNAse treated and loaded on a DEAE-packed column for Anion Exchange Chromatography. The vector-containing peak was collected, subjected to a second round of DNAse treatment, concentration by Tangential Flow Filtration and a final Size Exclusion Chromatography separation followed by sterilizing filtration and titration of the purified stock as previously described (Soldi (2020) Mol Therapy Methods).

### Gene Editing Protocol

For a 2-hit IDLV GE protocol, 12 h before electroporation 0.2-0.7×10⁶ cells were treated with 8 μM of cyclosporin H (CsH, Sigma) and then transduced with IDLV at MOI of 150. After 12 h, cells were washed with DPBS, electroporated as described above transduced with IDLV at MOI 150 for the second hit in presence of with 8 μM CsH. For 1-hit IDLV GE protocol, treatment with CsH (8 μM) and transduction with IDLV (MOI 150) were only performed 15 min after electroporation. GE protocol was performed using 5 μg of a combination of GSE56 mRNA and Ad5-E4orf6/7 mRNA as single RNA transfected along with RNP before AAV6 or IDLV transduction.

### Clonogenic Assay

Colony-forming-unit cell (CFU-C) assay was performed by plating in three technical replicates 800 HSPCs/each in methylcellulose based medium (MethoCult, STEMCELL Technologies) supplemented with 100 IU/ml penicillin and 100 μg/ml streptomycin. Two weeks after plating, colonies were counted and classified according to morphological criteria.

### Results

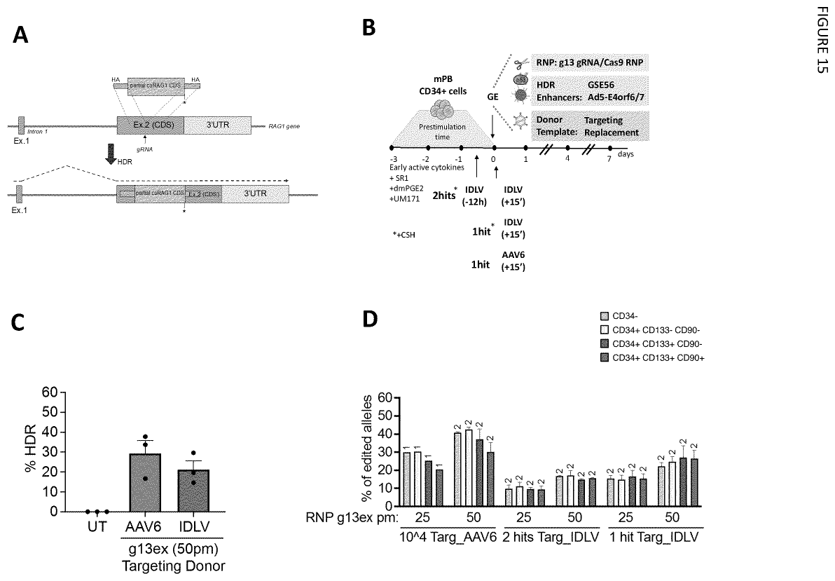

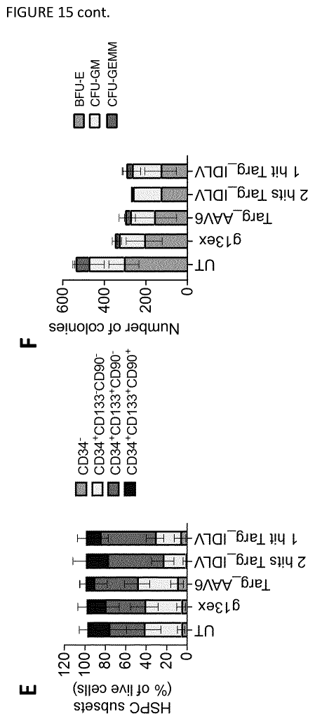

We have developed and tested a gene editing strategy targeting the second exon of RAG1 gene where all causative mutations have been described. As a corrective cassette we exploited a donor carrying the codon optimized human RAG1 coding sequence (co.hRAG1 CDS) in frame with the upstream portion of the endogenous hRAG1 and homology arms close to the exonic cutting site, that allows upon HDR to achieve a transcript including the distal region of the endogenous human RAG1 and the 3′UTR (FIG. 15A). We tested this platform exploiting AAV6 as a delivery vector and demonstrated high HDR-targeting in mPB-HSPCs and evidence of high specificity.

To support the clinical translation of GE and mitigate the genotoxic risks in HSPCs associated to the AAV6 platform, we exploited integrase-defective lentiviral vector (IDLV) as template delivery system for HDR editing, having previously demonstrated its superior cytotoxic and genotoxic profile. We cloned and produced the IDLV targeting donor (FIG. 15A) specific for g13ex target site and tested timing and rounds of IDLV transduction in HD-HSPCs (FIG. 15B). We compared IDLV transduction protocols (two hits and one hit protocol at 150 MOI) with 1 hit AAV6 protocol and selected the 1 hit protocol as the best performing in terms of HDR efficiency on bulk and primitive HSPCs (FIGS. 15C and D). Interestingly, a direct comparison of the optimized and selected GE protocols for AAV6 and IDLV platforms showed high and comparable levels of HDR-targeting in mPB-HSPCs (FIG. 15C). Despite a tendency to higher HDR efficiency in sorted HSPCs for AAV6 delivery platform than the IDLV-based GE, the difference flattened in the most primitive HSPCs (FIG. 15E) which were also more represented in IDLV-edited HSPCs (FIG. 15F). These data support the use of an IDLV-platform for the clinical translation of the exonic hRAG1 GE strategy.

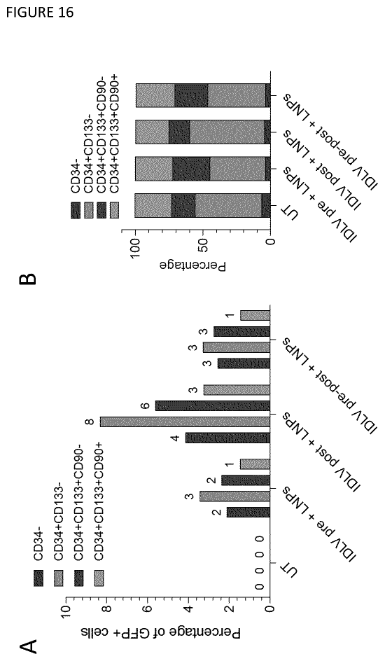

We also tested lipid nanoparticles (LNPs), together with the IDLV editing platform, as an alternative nuclease delivery modality to electroporation and confirmed editing efficiency in all HSPC subpopulations without phenotypic skewing (FIG. 16), confirming the feasibility of this approach.

1. Use of a combination of (a) cyclosporin H (CsH) or a derivative thereof, and (b) a p53 inhibitor and/or an adenoviral protein, or one or more nucleotide sequences encoding therefor, for increasing the efficiency of gene editing of an isolated population of cells when transduced by a viral vector and/or increasing the efficiency of transduction of an isolated population of cells by a viral vector.

2. A method of transducing a population of cells comprising the steps of:
i) contacting the population of cells with a combination of (a) cyclosporin H (CsH) or a derivative thereof, and (b) a p53 inhibitor and/or an adenoviral protein, or one or more nucleotide sequences encoding therefor; and
ii) transducing the population of cells with a viral vector.

3. The method according to claim 2, further comprising the step of iii) introducing a site-directed nuclease into the cells.

4. The method according to claim 3, wherein the site-directed nuclease is a CRISPR/Cas9 nuclease.

5. The use or method according to any preceding claim, wherein the combination comprises cyclosporin H (CsH) or a derivative thereof, a p53 inhibitor and an adenoviral protein.

6. The use or method according to any preceding claim, wherein the p53 inhibitor is a p53 dominant negative peptide.

7. The use or method according to any preceding claim, wherein the p53 inhibitor is GSE56 or a variant thereof; pifithrin-a or a derivative thereof; or an siRNA, shRNA, miRNA or antisense DNA/RNA.

8. The use or method according to any preceding claim, wherein the p53 inhibitor is GSE56 or a variant thereof.

9. The use or method according to any preceding claim, wherein the p53 inhibitor comprises or consists of an amino acid sequence having at least 70% sequence identity to SEQ ID NO: 3.

10. The use or method according to any preceding claim, wherein the adenoviral protein is from an Adenovirus of serotype 5.

11. The use or method according to any preceding claim, wherein the adenoviral protein is E4ORF6/7.

12. The use or method according to any preceding claim, wherein the adenoviral protein comprises or consists of an amino acid sequence having at least 70% to SEQ ID NO: 27.

13. The use or method according to any preceding claim, wherein the one or more nucleotide sequences are in the form of mRNA.

14. The use or method according to any preceding claim, wherein the viral vector is an Integration-Defective Lentiviral Vector (IDLV).

15. The use or method according to any preceding claim, wherein the cells are haematopoietic stem and/or progenitor cells (HSPCs).

16. The use or method according to any preceding claim, wherein the cells are T cells.

17. The use or method according to any preceding claim, wherein the cells are contacted with cyclosporin H prior to and/or at the same time as contact with the viral vector.

18. The use or method according to any preceding claim, wherein the cells are contacted with a p53 inhibitor and/or an adenoviral protein, or one or more nucleotide sequences encoding therefor, prior to and/or at the same time as contact with a site-directed nuclease.

19. The method according to any one of claims 3 to 18, wherein contact with the site-directed nuclease occurs about 10-14 hours, optionally about 12 hours after the beginning of the transduction step.

20. The use or method according to any preceding claim, wherein the population of cells is transduced by the viral vector in a single transduction step or in two transduction steps.

21. A combination of (a) cyclosporin H (CsH) or a derivative thereof, and (b) a p53 inhibitor and/or an adenoviral protein, or one or more nucleotide sequences encoding therefor, for use in a method of gene editing.

22. A population of transduced cells, prepared according to the method of any one of claims 2 to 20.

23. A pharmaceutical composition comprising the population of transduced cells of claim 22.

24. A method of treating or preventing a disease in a subject, comprising administering the population of cells according to claim 22, or composition according to claim 23, to the subject.

25. A population of cells according to claim 22, or a composition according to claim 23, for use in a method of treating or preventing a disease in a subject.

26. A method of gene therapy comprising the steps:
(a) transducing a population of cells according to the method of any one of claims 2 to 20; and
(b) administering the transduced cells to a subject.
